(19) United States
(12) Patent Application Publication
(10) Pub. No.: US 2024/0180981 A1
(43) Pub. Date: Jun. 6, 2024

(54) METHODS TO ANALYZE HOST-MICROBIOME INTERACTIONS AT SINGLE-CELL AND ASSOCIATED GENE SIGNATURES IN CANCER

(71) Applicant: Rutgers, The State University of New Jersey, New Brunswick, NJ (US)

(72) Inventors: Bassel Ghaddar, Highland Park, NJ (US); Subhajyoti De, Princeton Junction, NJ (US)

(73) Assignee: Rutgers, The State University of New Jersey, New Brunswick, NJ (US)

(21) Appl. No.: 18/287,763

(22) PCT Filed: Apr. 21, 2022
(86) PCT No.: PCT/US2022/025832

§ 371 Date: Oct. 20, 2023

Related U.S. Application Data
(60) Provisional application No. 63/177,808, filed on Apr. 21, 2021.

(51) Int. Cl. A61K 35/76; A61K 31/282; A61K 31/337; A61K 31/4745; A61K 31/513; A61K 31/7068; A61K 33/243; A61K 47/64; A61P 35/00; C12Q 1/6886; C12Q 1/689; C12Q 1/70

(52) U.S. Cl. CPC A61K 35/76; A61K 31/282; A61K 31/337; A61K 31/4745; A61K 31/513; A61K 31/7068; A61K 33/243; A61K 47/643; A61P 35/00; C12Q 1/6886; C12Q 1/689; C12Q 1/70; C12Q 2600/118; C12Q 2600/158

(57) ABSTRACT

Disclosed herein are methods of identifying and treating subjects with cancer, and methods of predicting a survival outcome in a subject with cancer, such as pancreatic cancer. In one aspect, the application provides methods for detecting the presence of cancer or infectious disease in a subject by collecting and analyzing sequencing information from the subject, such as by performing single cell RNA sequencing analysis of individual cells obtained from a sample from the subject. In a further aspect, the application provides methods for detecting the presence of cancer or infectious disease in a subject by determining microbial diversity and/or assessing the presence or absence of particular microbes in individual cells from the subject as compared to a control. Also provided are methods of determining T-cell microenvironment reaction, for example by sequencing nucleic acid molecules in individual T-cells obtained from the subject.

### CROSS-REFERENCE TO RELATED APPLICATIONS

This application claims priority to U.S. Provisional Application No. 63/177,808, filed Apr. 21, 2021, which is herein incorporated by reference in its entirety.

### ACKNOWLEDGMENT OF GOVERNMENT SUPPORT

This invention was made with government support under Contract number R21 CA248122 awarded by the National Institutes of Health. The government has certain rights in the invention.

### FIELD

This disclosure relates to microbial signatures for prediction of cancer patient outcomes, and methods of their use, including methods for treating cancer in a subject, as well as methods of identifying an infection in a subject.

### BACKGROUND

The microbiome contributes to numerous aspects of human health and disease, including oncogenesis. While it is uncertain whether the healthy pancreas harbors its own microbiome, emerging evidence indicates that bacteria and fungi can translocate to the pancreas and induce local and systemic changes that promote the development of pancreatic ductal adenocarcinoma (PDA) (Vitiello et al. Trends in Cancer, 5:670-676, 2019; Wei et al. Mol. Cancer 18:1-15, 2019). Microbiota products alter gene regulation (Yoshimoto et al. Nature, 499:97-101, 2013) and lead to DNA damage (Öğrendik, Gastrointest. Tumors, 3:125-127, 2017), stimulate pattern recognition receptors that potentiate mutant KRAS signaling (Ochi et al. J. Exp. Med. 209:1671-1687, 2012; Zambirinis et al. Cell Cycle, 12: 1153-1154, 2013), and can induce both inflammation and immunosuppression (Pushalkar et al. Cancer Discov. 8: 403-416, 2018; Zambirinis et al. J. Exp. Med. 212: 2077-2094, 2015; Aykut et al. Nature, 574: 264-267, 2019; Seifert et al. Nature, 532: 245-249, 2016. Microbiota within PDA also may confer resistance to therapies, including deactivating gemcitabine via microbial cytidine deaminase (Geller et al. Science, 357(6356):1156-1160, 2017)., while antibiotic-induced reduction of the gut microbiome may increase sensitivity to immune checkpoint inhibitors (Pushalkar et al. Cancer Discov,. 8: 403-416 2018; Sethi et al. Gastroenterology, 155: 33-37.e6, 2018; Thomas et al. Carcinogenesis, 39: 1068-1078, 2018).

Several barriers limit the systematic investigation of the microbiome in PDA patients (Sethi et al. Gastroenterology, 156: 2097-2115.e2, 2019). First, many intestinal microbes are difficult to culture in vivo (Suau et al. Appl. Environ. Microbiol. 65(11):4799-807, 1999). Second, microbiome composition can differ vastly (Ericsson et al. PLOS One, 10: e0116704, 2015; De Filippo et al. Proc. Natl. Acad. Sci. 107(33): 14691-6, 2010; Nguyen et al. Dis. Model. Mech. 8(1): 1-16, 2015), and there are few model systems that sufficiently recapitulate tumor-microbiome interactions in humans (Mallapaty, Lab Anim. 46: 373-377, 2017; Saluja et al. Gastroenterology, 144: 1194-1198, 2013). Third, the possibility of sample contamination post-surgery complicates data interpretation (de Goffau, et al. Nat. Microbiol. 3: 851-853, 2018; Zinter et al. Microbiome, 7: 1-5, 2019). Recently, using The Cancer Genome Atlas (TCGA), (Poore et al. Nature, 579: 567-574, 2020) discovered cancer-type specific microbial signatures, and (Nejman et al. Science, 368(6494): 973-980, 2020) identified tumor-specific intracellular bacteria through 16S rRNA profiling of hundreds of tumors. However, these studies analyzed genomic data from bulk tissue samples, which do not capture microbial-somatic cell enrichments, associations with cell-type specific activities, or microbial contributions to inter-cellular communication networks. In particular, PDA is characterized by a fibrotic stroma comprising the majority of tumor volume, which makes disentangling cellular relationships difficult by bulk profiling (Moffitt et al. Nat. Genet. 47: 1168-1178, 2015). As a result, the inventors develop SAHMI (Single-cell Analysis of Host-Microbiome Interactions) to examine patterns of human-microbiome interactions in the pancreatic tumor microenvironment at single cell resolution using genomic approaches.

### SUMMARY

Methods of identifying and treating subjects with cancer, and methods of predicting a survival outcome in a subject with cancer are disclosed herein. In some embodiments, the disclosed methods include detecting the presence of cancer in a subject by sequencing microbial nucleic acid molecules in individual cells obtained from the subject and comparing expression levels in the individual cells to a control. In some examples sequencing and quantifying of nucleic acids from the individual cells (such as individual pancreatic cells, such as normal and/or tumor pancreatic cells) is achieved by performing single cell RNA sequencing (scRNA-seq) analysis. In such methods, the subject is diagnosed as having pancreatic cancer when the presence of Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, and/or Aspergillus microbes is detected in the tumor (either intra-or extra-cellularly in, e.g., pancreatic tumors) at an elevated abundance compared to analogous healthy tissues (e.g., healthy pancreatic tissues) and/or when the presence of Staphylococcus, Paraccocus, Burkholderia, Klebsiella, Pasteurella, and/or Ralstonia microbes is detected in the tumor (either intra- or extra-cellularly in, e.g., pancreatic tumors) at a decreased abundance compared to analogous healthy tissues (e.g., healthy pancreatic tissues).

The disclosed methods also include treating a subject having or suspected of having pancreatic cancer. In such examples, microbial nucleic acid molecules in individual cells (such as individual pancreatic cells, such as normal and/or tumor pancreatic cells) obtained from the subject are sequenced, and the subject is diagnosed as having pancreatic cancer when the presence of Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, and/or Aspergillus microbes is detected in the tumor (either intra-or extra-cellularly in, e.g., pancreatic tumors) at an elevated abundance compared to analogous healthy tissues (e.g., healthy pancreatic tissues) and/or when the presence of Staphylococcus, Paraccocus, Burkholderia, Klebsiella, Pasteurella, and/or Ralstonia microbes is detected in the tumor (either intra- or extra-cellularly in, e.g., pancreatic tumors) at a decreased abundance compared to analogous healthy tissues (e.g., healthy pancreatic tissues). A subject who is diagnosed as having pancreatic cancer can be treated using at least one of surgery, radiation therapy, chemotherapy, administration of an antimicrobial, administration of a selective bacteriophage, or palliative care.

Disclosed methods further include methods of predicting a survival outcome of subjects with pancreatic cancer. In such examples, microbial nucleic acid molecules in individual cells (such as individual pancreatic cells, such as normal and/or tumor pancreatic cells) obtained from the subject are sequenced (such as by scRNA-seq), and the subject is classified as having a poor survival outcome when the presence of Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, and/or Aspergillus microbes is detected in the tumor (either intra-or extra-cellularly in, e.g., pancreatic tumors) at an elevated abundance compared to analogous healthy tissues (e.g., healthy pancreatic tissues) and/or when the presence of Staphylococcus, Paraccocus, Burkholderia, Klebsiella, Pasteurella, and/or Ralstonia microbes is detected in the tumor (either intra- or extra-cellularly in, e.g., pancreatic tumors) at a decreased abundance compared to analogous healthy tissues (e.g., healthy pancreatic tissues). In other embodiments of the method, survival outcome in a subject with pancreatic cancer is predicted based on expression (as measured in cells isolated from a sample from the subject and, in certain embodiments, compared to a control) of a set of genes including NTHL1, LYPD2, MUC16, C2CD4B, FMO3, and/or IL1RL1. In specific examples, increased expression of one or more of IL1RL1, C2CD4B, FMO3, or NTHL1 compared to a control, and/or decreased expression of one or more of LYPD2 or MUC16 compared to the control indicates high microbial diversity in the sample, and classifies the subject as having a poor survival outcome.

Methods of determining T-cell microenvironment reaction in a subject are also disclosed. In such an embodiment, nucleic acid molecules (such as one or more of those in Table 2) in individual T-cells obtained from the subject are sequenced, such as by scRNA-seq. Expression levels of one or more genes in the individual T-cells are determined and compared to a control, thereby classifying the individual T-cells having a transcriptional phenotype classified as either a tumor microenvironment reaction or infection microenvironment reaction.

Methods of identifying a microbe or virus in a sample are also disclosed. In such an embodiment, nucleic acid molecules in individual cells obtained from the sample (such as from a sample from a subject) are sequenced, such as by scRNA-seq; and the microbe or virus is identified when a microbial or viral nucleic acid indicative of the presence of the microbe or the virus is detected . In some embodiments, the identifying includes mapping reads from a single cell RNA sequencing dataset for the sample to microbial and/or viral genomes using a metagenomics classifier, thereby assigning a genus and/or species identity to each read in the dataset. For each genus and/or species identified, the number of reads assigned and the number of minimizers assigned are compared, the number of minimizers assigned and the number of unique minimizers assigned are compared; and the number of reads assigned and the number of unique minimizers assigned are compared. The genus and/or species identified is classified as a true positive result when a correlation value for each of the three comparisons is positive, and when a number of reads detected for the species is greater in the single cell RNA sequencing dataset for the sample as compared to a control. In some embodiments, the control is a sample from a subject or a group of subjects that does not have the infection, or a sample from at least one cell line that does not have the infection.

Methods of treating a subject having or suspected of having an infectious disease caused by a microbe or a virus are also disclosed. In such an embodiment, nucleic acid molecules in individual cells obtained from a sample from the subject are sequenced, such as by scRNA-seq, and the subject is classified as having the infectious disease when a microbial or viral nucleic acid indicative of the presence of the microbe or the virus is detected in the individual cells. In some embodiments, the identifying includes mapping reads from a single cell RNA sequencing dataset for the sample to microbial and/or viral genomes using a metagenomics classifier, thereby assigning a genus and/or species identity to each read in the dataset. For each genus and/or species identified, the number of reads assigned and the number of minimizers assigned are compared, the number of minimizers assigned and the number of unique minimizers assigned are compared; and the number of reads assigned and the number of unique minimizers assigned are compared. The genus and/or species identified is classified as a true positive result when a correlation value for each of the three comparisons is positive, and when a number of reads detected for the species is greater in the single cell RNA sequencing dataset for the sample as compared to a control. In some examples, if the subject is determined to have the infectious disease, the subject is administered at least one of an antibiotic, antifungal, or antiviral, thereby treating the subject. In some embodiments, the control is a sample from a subject or a group of subjects that does not have the infection, or a sample from at least one cell line that does not have the infection.

Methods of diagnosing a subject with an infectious disease caused by a microbe or a virus are also disclosed. In such an embodiment, nucleic acid molecules in individual cells obtained from the subject are sequenced, such as by scRNA-seq, and the subject is classified as having the infectious disease when a microbial or viral nucleic acid indicative of the presence of the microbe or the virus is detected in the individual cells. In some embodiments, the detecting includes mapping reads from a single cell RNA sequencing dataset for the sample to microbial and/or viral genomes using a metagenomics classifier, thereby assigning a genus and/or species identity to each read in the dataset. For each genus and/or species identified, the number of reads assigned and the number of minimizers assigned are compared, the number of minimizers assigned and the number of unique minimizers assigned are compared; and the number of reads assigned and the number of unique minimizers assigned are compared. The genus and/or species identified is classified as a true positive result when a correlation value for each of the three comparisons is positive, and when a number of reads detected for the species is greater in the single cell RNA sequencing dataset for the sample as compared to a control. In some embodiments, the control is a sample from a subject or a group of subjects that does not have the infection, or a sample from at least one cell line that does not have the infection.

The foregoing and other features of the disclosure will become more apparent from the following detailed description, which proceeds with reference to the accompanying figures.

### BRIEF DESCRIPTION OF THE DRAWINGS

FIGS. 1A-1G show detection and validation of a distinct and diverse PDA microbiome. (FIG. 1A) Study design. See also Table 1. PDA, pancreatic ductal adenocarcinoma. (FIG. 1B) Differential abundances of microbial changes in pancreatic disease and in previously reported putative laboratory contaminants; boxplots show median (line), 25^{th} and 75^{th} percentiles (box) and 1.5×IQR (whiskers). Points represent outliers. N=nonmalignant tissues (n=11), T=tumors (n=24) (Wilcoxon test, ns=p>0.05, *p<0.05, **p<0.01, ***p<0.001, ****p<0.0001). (FIG. 1C) Comparisons of bacterial abundance in pancreatic tissues across multiple studies using differing technologies. Lower triangle=Spearman correlation of study-level abundances, upper triangle=overlap coefficient of present/absent genera. Columns indicate the number of samples, and rows the number of genera passing quality filters. scRNAseq=single-cell RNA sequencing, TCGA=The Cancer Genome Atlas. (FIG. 1D) Bar plots of relative abundances of genera in the Peng cohort. (FIG. 1E) Differentially present bacterial and fungal genera in nonmalignant vs. tumor samples computed from a linear model with tissue status, total metagenomic counts, and sample composition as covariates. Data shown for genera with abundance>10⁻³ or those listed in FIG. 1B. DE Coef, differential expression coefficient, Q, adjusted-p value. (FIG. 1F) Uniform manifold approximation and projection (UMAP) of barcodes tagging bacterial (left, n=23,4466 barcodes) and fungal (right, n=4,312 barcodes) DNA, colored by tissue status (N, nonmalignant, T, tumor). (FIG. 1G) Alpha-diversity of nonmalignant (N) and tumor (T) microbiomes, based in Shannon and Simpson scores. Box plots are as above, with Wilcoxon testing.

FIGS. 2A-2G show that microbes are associated with particular host cells and correlate with immune infiltration and diversity. (FIG. 2A) UMAP of barcodes tagging bacterial (left, n=23,4466 barcodes) and fungal (right, n=4,312 barcodes) DNA, colored by associated somatic-cell type. (FIG. 2B) Circos-plot of significant microbe-somatic cell enrichments identified at the single-barcode level by Wilcoxon testing. The ribbon width correlates with enrichment strength. (FIG. 2C) Statistically significant microbe-somatic cell enrichments in subsampled vs. cell-type label-shuffled (random) data in two data sets of scRNAseq, and the number of enrichments shared between the two studies. Two distributions were compared by applying Wilcoxon test. Bars, mean number of enrichments, Error-bars, bootstrapped 95% confidence intervals. (FIG. 2D) ROCs for random forest predictions of barcode cell-types using microbiome profiles alone. Curves colored by cell type. AUC, area under the curve. (FIG. 2E) Somatic cellular composition prediction using 34 sample-level microbiome abundances. Each point represents a normalized cell-type level in sample, colored as in FIG. 2D. (FIG. 2F) Self-assembling manifold (SAM) principal component analysis for individual somatic-cell types based on transcriptome. Cells colored by their data-driven cluster assignment, with immune types annotated: GC, germinal center, DC, dendritic cell, MP, macrophage, Th 17, T-helper 17, TCM, T-central memory, TEM, T-effector memory, Treg, T-regulatory, Tfh, T-follicular helper, NK, natural killer. (FIG. 2G) Spearman correlations of microbial (Shannon) diversity and somatic cellular fraction (top) or somatic cellular diversity (bottom) in the same sample. Somatic cell diversity was calculated using cluster assignments from FIG. 2F. TME, tumor microenvironment.

FIGS. 3A-3H show that specific microbe abundances correlate with co-localized cell-type specific gene expression. (FIG. 3A) Unsupervised dot-plots represent significant correlations between normal and tumor-specific microbes and receptor gene expression in their co-localized cell-types: Rows, differentially expressed microbe genera from FIG. 1E; columns, receptor gene expression levels; triangles, positive, circle, negative correlation. Colors represent the cell-type for the correlation. Boxes added to highlight significant clusters, with significant KEGG-pathway enrichments indicated. (FIG. 3B) Volcano plots for correlations between individual microbe abundances and gene expression (top, individual cells) or pathway scores (bottom, averaged cell-type scores), colored by point density. (FIG. 3C) Heatmap of Spearman correlations between sample-level microbial abundances and inflammation-related gene expression. (FIG. 3D) Network of microbe-cell-specific pathway and pathway-pathway associations. Nodes represent either microbe or cell-specific pathway score, with edges linking nodes with significant correlations (|r|>0.5, p<0.05). Nodes are colored by cell-type and shaped by their pathway category: Blue edges, negative correlation. See also FIG. 9. (FIG. 3E) Edge centrality computed from FIG. 3D. Colors based on node linkages connecting a microbe (orange) or only connecting somatic pathways (grey). (FIG. 3F) Linkage of bacterial abundances and gene expression in Peng and TCGA samples. Bacteroides and LYZ gene expression and (FIG. 3G) Campylobacter and Hippo signaling. (FIG. 3H) Number of statistically significant, shared microbe-gene or pathway associations between the Peng cohort (Peng et al. Cell Res. 29(9):725-738, 2019) and TCGA (Poore et al. Nature 579: 567-574, 2020) in subsampled vs. sample-label shuffled data. Bars, mean number of enrichments, Error-bars, bootstrapped 95% confidence intervals (n=500, Wilcoxon-test).

FIGS. 4A-4C show microbe abundances that correlate with cell-type specific pathway activity scores. Unsupervised dot-plots representing biologically and statistically significant Spearman correlations (|r|>0.5, p<0.05, t-test) between normal and tumor-specific microbes and pathways in their co-localized cell-types. Key: Rows, differentially expressed microbe genera (FIG. 1E); Columns, KEGG pathways; Triangles, positive, Circle, negative correlation; Colors, cell-type (FIG. 2F) in which the correlation existed. (FIG. 4A, FIG. 4B) Non-metabolic pathways; (FIG. 4C) metabolic pathways.

FIGS. 5A-5H show T-cell characteristics, microenvironment features and microbiome-clinical associations. (FIG. 5A) Training and test datasets used to create a random forest model to distinguish between T-cells infection vs. tumor microenvironment reaction based on their gene expression profiles. (FIG. 5B) ROC curve indicating exceptional model performance on test datasets; AUC, area under the curve. Inset: Confusion matrix of model assignments; rows, predicted, columns, true values. (FIG. 5C) Bar-plot of predicted T-cell microenvironment reaction in the Peng cohort. (FIG. 5D) Pseudotime analysis of samples based on microbiome profiles and cell-specific pathway scores identifies distinct states: NS, normal state, TS, tumor state representing data-driven PDA subtypes with distinct molecular, microbiome, and clinical characteristics. Arrows indicate microbiome and clinical differences amongst TS1-3, based on t-tests and Fisher's test. (FIG. 5E) Circular heatmap of microbiome/pathway differences for the four states. Rows represent microbe or cell-specific pathway; Columns represent the four states, with NS outermost, followed by TS1, 2, 3. Average microbe expression or pathway score: Red, high; Blue, low. (FIG. 5F) Example pathway and microbiome changes in the four states as samples progress along pseudotime. Points represent individual samples colored by their state. (FIG. 5G) Confusion matrix showing the utility of a 6-gene signature in classifying Peng (Peng et al. Cell Res. 29(9): 725-738, 2019) samples as high or low microbiome diversity. (FIG. 5H) Kaplan-Meier plots of TCGA (left) and ICGC PDA (center) cohorts stratified by predicted microbial diversity, and (right) survival curves for TCGA PDA cohorts stratified by microbiome diversity directly measured from the same samples by (Poore et al. Nature. 579: 567-574, 2020) (TCGA observed).

FIGS. 6A-6G show quality measures and metagenomic read statistics. (FIG. 6A) Uniform manifold approximation and projection (UMAP) of somatic cells clustered by transcriptomes profiles and colored by sample type (left panel, N=nonmalignant, T=tumor), patient sample (middle panel), and cell-type (right panel). (FIG. 6B) Percent of bacterial reads resolved to the genus level that were discarded due to being PCR duplicates, having low genera abundance, or not passing the multi-study filter. The remaining reads were retained for downstream analysis. (FIG. 6C) Processed metagenomic vs. somatic gene counts; N=nonmalignant, T=tumor. (FIG. 6D) Boxplots of metagenomic read counts in nonmalignant (N) and tumor (T) samples showing median (line), 25th and 75th percentiles (box) and 1.5×IQR (whiskers). (FIG. 6E) Boxplots showing metagenomic counts per cell type in nonmalignant (N) and tumor (T) samples. Inset: Percentage of metagenomes that are somatic cell-associated in nonmalignant (N) and tumor (T) samples.

Boxplots show median (line), 25th and 75th percentiles (box) and 1.5×IQR (whiskers). (FIG. 6F) UMAP plot of metagenomic barcodes from three pancreas single-cell RNA sequencing datasets colored by study of origin. Peng N=nonmalignant Peng samples, Peng T=tumor Peng samples. (FIG. 6G) UMAP plot of bacterial and fungal metagenomic barcodes from the Peng cohort. Red=barcodes from tumors, blue=barcodes from nonmalignant samples, circles=bacteria-only barcodes, squares=fungi-only barcodes, triangles=bacteria and fungi barcodes.

FIGS. 7A-7B shows cell-type and sample cellular composition predictions with null models. (FIG. 7A) Sensitivity vs. specificity curves for random forest predictions of label-shuffled barcode cell-types using barcode metagenomic profiles. Curves are colored by cell type. AUC, area under the curve. (FIG. 7B) Distribution of R-squared values from 100 null models using 34 sample-level abundances to predict sample somatic cellular composition. Null models were created by shuffling sample labels.

FIGS. 8A-8E show microbiome associations with numerous somatic cellular activities. (FIG. 8A) Ranked pathway enrichments from biologically and statistically significant (|r|>0.5, p<0.05) microbe-gene pathway correlations in individual cells. (FIG. 8B) Heatmap showing Spearman correlation coefficients between microbes and total antimicrobial gene expression. (FIG. 8C) Volcano plot of microbe- pathway correlations between all average cell-type specific microbe levels and cell-type specific pathways. (FIG. 8D) Heatmap showing Spearman correlation coefficients for significant correlations from FIG. 8C with |r|>0.5 and p<0.05 for pathways involving malignant ductal 2 cells. (FIG. 8E) Heatmap showing correlations from FIG. 8C with |r|>0.5 and p<0.05 for all pathways and cell-types.

FIG. 9 shows a network of correlations between microbes and cell-type specific cancer-related pathway scores. Nodes represent either a microbe or cell-type specific pathway. Edges represent a significant correlation between nodes, defined as |r|>0.5 and p<0.05 for microbe-pathway correlations, and |r|>0.75 and p<0.05 for pathway-pathway correlations. A higher cutoff was used for pathway-pathway correlations to account for overlapping gene sets in some pathways. Nodes are colored by their somatic or microbial cell-type, shaped by their pathway category (or otherwise microbe), and sized proportionally to their number of edges. Grey edges represent positive correlations, and blue edges represent negative correlations.

FIG. 10 shows a pseudotime analysis of tumor microenvironments using pathway scores alone. Average cell-type specific pathway scores for cancer-related pathways were used to order entire tumor microenvironments along a progressive process. The same branching pattern with distinct clusters emerges as when microbiome profiles are included (see FIG. 5D).

FIG. 11 shows detection of known infections using scRNA-seq data from a variety of tissue types and pathogens. Box plots show read counts per million assigned microbiome reads for infected versus uninfected samples in multiple benchmark datasets with either a known pathogen (either introduced or clinically identified). Boxplots show the median (horizontal line), 25th and 75th percentiles (box), and 1.5× the interquartile range (IQR) (whiskers) for each experiment. Points represent outliers. Statistical significance was determined using Wilcoxon testing (p<0.001).

FIGS. 12A-12D shows criteria for detecting and de-noising microbiome signals. (FIG. 12A) Sequencing reads from true species have positive relationships between (1) the number of reads assigned and number of minimizers assigned, (2) number of minimizers assigned and number of unique minimizers assigned, and (3) number of reads assigned and number of unique minimizers assigned. Data are shown for the benchmark datasets tested. (FIG. 12B) Table detailing benchmark dataset metadata and Spearman correlation coefficients from FIG. 12A. (FIG. 12C) Scatter plot showing the relationship between the three correlations from FIG. 12A for all species detected in the benchmark datasets. Each point represents a species. Extension of the cloud of points into low correlation values indicates the presence of abundant false positive results. Concentration of points at high values suggest the presence of other species, including contaminants. (FIG. 12D) Scatter plot showing the relationship between the three correlations in FIG. 12A for microbiomes detected in cell line experiments taken as benchmark negative controls. Any species shown in this scatter plot are contaminants or false positives. In test samples, species not detected above the thresholds found in negative controls were assumed to be false positive or contaminant species.

### DETAILED DESCRIPTION

### I. Terms

Unless otherwise explained, all technical and scientific terms used herein have the same meaning as commonly understood by one of ordinary skill in the art to which this disclosure belongs. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

The singular terms “a,” “an,” and “the” include plural referents unless the context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank Accession numbers. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

About: Unless context indicated otherwise, “about” refers to plus or minus 5% of a reference value. For example, “about” 100 refers to 95 to 105.

Administration/delivery: To provide or give a subject an agent or therapy by any chosen route. Examples of agents include chemotherapy, surgery, radiation therapy, targeted therapy, antimicrobial therapy (e.g., one or more antibiotics and/or antifungals), immunotherapy, or palliative care. Administration includes acute and chronic administration as well as local and systemic administration. In some examples, administration of a therapeutic agent, such as chemotherapy, is by injection (e.g., intravenous, intramuscular, subcutaneous, intradermal, intrathecal (such as lumbar puncture), intraosseous, intratumoral, intrapancreatic, or intraperitoneal). In some examples, administration of a therapeutic agent, such as chemotherapy, is oral (such as sublingual), rectal, transdermal (such as topical), intranasal, vaginal, or by inhalation.

Animal: Living multi-cellular vertebrate organisms, a category that includes, for example, mammals and birds. The term mammal includes both human and non-human mammals. Similarly, the term “subject” includes both human and veterinary subjects.

Chemotherapeutic agent or Chemotherapy: Any chemical or biological agent with therapeutic usefulness in the treatment of diseases characterized by abnormal cell growth. Such diseases include tumors, neoplasms, and cancer. In one embodiment, a chemotherapeutic agent is an agent of use in treating cancer, such as lung or pancreatic cancer, such as PDA. In some examples, chemotherapeutic agents include gemcitabine, 5-fluorouracil, oxaliplatin, capecitabine, cisplatin, irinotecan, liposomal irinotecan, paclitaxel, albumin-bound paclitaxel, or docetaxel, carboplatin, vinorelbine, folinic acid, or oxaliplatin, in any combination together or with other agents. In some examples, the chemotherapeutic agents include a combination of carboplatin and paclitaxel, a combination of cisplatin and vinorelbine, and a combination of folinic acid, fluorouracil, and oxaliplatin. Exemplary chemotherapeutic agents are provided in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993, all incorporated herein by reference. Combination chemotherapy is the administration of more than one agent (such as more than one chemical chemotherapeutic agent) to treat cancer. Such a combination can be administered simultaneously, contemporaneously, or with a period of time in between.

In one example, a chemotherapy treatment for a pancreatic cancer analyzed using the disclosed methods includes gemcitabine, 5-FU, or capecitabine, such as fluorouracil, leucovorin, irinotecan, and oxaliplatin, (FOLFIRINOX). In one example, a chemotherapy treatment for a pancreatic cancer analyzed using the disclosed methods includes gemcitabine plus nab-paclitaxel. In one example, a chemotherapy treatment for a pancreatic cancer analyzed using the disclosed methods includes gemcitabine

Control: A reference standard. In some embodiments, the control is a healthy subject. In other embodiments, the control is a subject with a cancer, such as a pancreatic cancer. In some embodiments, the control is a subject who responds positively to chemotherapy, such as a subject who does not develop resistance to chemotherapy. In other embodiments, the control is a subject who does not respond positively to chemotherapy, such as a subject who develops resistance to chemotherapy. In some embodiments, the control is tissue sampled from a subject, such as healthy tissue sampled from a subject having a cancer, such as healthy pancreatic tissue sampled from a subject having pancreatic cancer, wherein a pancreatic cancer tissue sample is also taken from the same subject. In still other embodiments, the control is a historical control or standard reference value or range of values (e.g., a previously tested control subject with a known prognosis or outcome or group of subjects that represent baseline or normal values). A difference between a test subject and a control can be an increase or a decrease. The difference can be a qualitative difference or a quantitative difference, for example a statistically significant difference.

Detect: To determine if an agent (such as a signal; particular nucleotide; amino acid; nucleic acid molecule and/or nucleotide modification, such as a methylated nucleotide; mRNA; or protein) is present or absent. In some examples, detection can include further quantification. For example, use of the disclosed methods (such as single cell RNA sequencing) in particular examples permits detection of nucleic acid expression (e.g., mRNA levels) in a sample.

Differential Expression: A nucleic acid molecule is differentially expressed when the amount of one or more of its expression products (e.g., transcript, such as mRNA, and/or protein) is higher or lower in one sample (such as a test pancreatic cancer sample) as compared to another sample (such as a control pancreatic cancer sample). Detecting differential expression can include measuring a change in gene (such as by measuring mRNA) or protein expression. An exemplary gene expression measurement method is RNA sequencing, such as single cell RNA sequencing. Protein expression is translation of a nucleic acid into a peptide or protein. Peptides or proteins may be expressed and remain intracellular, become a component of the cell surface membrane, or be secreted into the extracellular matrix or medium.

Pancreatic cancer: A malignant tumor within the pancreas. The prognosis is generally poor. About 95% of pancreatic cancers are adenocarcinomas. The remaining 5% are tumors of the exocrine pancreas (for example, serous cystadenomas), ascinar cell cancers, and pancreatic neuroendocrine tumors (such as insulinomas). A pancreatic adenocarcinoma occurs in the glandular tissue. Symptoms include abdominal pain, loss of appetite, weight loss, jaundice and painless extension of the gallbladder. Exemplary treatment for pancreatic cancer, including adenocarcinomas and insulinomas includes surgical resection (such as the Whipple procedure) and administration of one or more chemotherapy agents, such as one or more of fluorouracil, gemcitabine, 5-FU, and erlotinib.

Sample or biological sample: A sample of biological material obtained from a subject, which can include cells, proteins, and/or nucleic acid molecules (such as DNA and/or RNA, such as mRNA). Biological samples include all clinical samples useful for detection of disease, such as cancer (such as pancreatic cancer), in subjects. Appropriate samples include any conventional biological samples, including clinical samples obtained from a human or veterinary subject. Exemplary samples include, without limitation, cancer samples (such as from surgery, tissue biopsy, tissue sections, or autopsy), cells, cell lysates, blood smears, cytocentrifuge preparations, cytology smears, bodily fluids (e.g., blood, plasma, serum, stool/feces, saliva, sputum, urine, bronchoalveolar lavage, semen, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In a particular example, a sample or biological sample is obtained from a subject having, suspected of having, or at risk of having cancer (such as pancreatic cancer). In a specific example, the sample is a pancreatic cancer sample. In a specific example, the sample is a non-cancerous pancreatic sample, for example from the same pancreases that is cancerous). In another specific example, the sample is a lung cancer sample. In further examples, the sample is from a subject having, suspected of having, or at risk of having an infectious disease.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, MD 20894) and on the Internet, for use in connection with the sequence analysis programs blastp, blastn, blastx, tblastn, and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs may use SEG filtering (Wootton and Federhen, Meth. Enzymol. 266:554-571, 1996). In addition, a manual alignment can be performed.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 80%, at least 90%, at least 95%, at least 98%, or at least 99% sequence identity to a nucleic acid molecule sequenced using the disclosed methods . An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Shannon Diversity Index: The Shannon diversity index (H) is a mathematical measure that is used to characterize species diversity in a community, and accounts for both species richness (the number of species present) and evenness (relative abundances of different species) present in the community. Most often, the proportion of species i relative to the total number of species (pᵢ) is calculated and multiplied by the natural logarithm of the proportion (lnpᵢ). The result is then summed across species and multiplied by −1:

H=−Σᵢ₌₁^{k}pᵢ log(pᵢ)

Further, Shannon's equitability (E_{H}) is determined by dividing H by the maximum diversity (log(k)). This normalizes the Shannon diversity index to a value between 0 and 1, with 1 being complete evenness of species in the community. In other words, an index value of 1 means that all species groups have the same frequency.

$${E}_{H} = \frac{H}{log ( k )}$$

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In one example, the subject treated and/or analyzed with the disclosed methods has cancer, such as pancreatic or lung cancer. In some examples, the subject has not been diagnosed with a cancer, but is suspected of having a cancer, such as a pancreatic cancer.

T-Cell and T-Cell Reactivity: A white blood cell critical to the immune response. T-cells include, but are not limited to, CD4+ T-cells and CD8+ T-cells. A CD4+ T lymphocyte is an immune cell that carries a marker on its surface known as “cluster of differentiation 4” (CD4). These cells, also known as helper T-cells, help orchestrate the immune response, including antibody responses as well as killer T-cell responses. In another embodiment, a CD4+ cell is a regulatory T-cell (Treg). CD8+ T-cells carry the “cluster of differentiation 8” (CD8) marker. In one embodiment, a CD8 T-cell is a cytotoxic T lymphocyte. An effector function of a T-cell is a specialized function of the T-cell, such as cytolytic activity or helper activity including the secretion of cytokines. A mature T-cell is a T-cell that is CD3+CD4+CD8− or CD3+CD4−CD8+. “T-cell microenvironment reaction” refers to T-cells (such as T-cells that are isolated from a sample from a subject) that are classified using expression analyses (such as sc-RNAseq) as either tumor-microenvironment transcriptional response (and can indicate what fraction of a sample's T-cells are responding to tumor-related signals) or infection microenvironment transcriptional response (and can indicate what fraction of a sample's T-cells are responding to infection-related signals).

Therapeutically effective amount: The amount of an active ingredient (such as a chemotherapeutic agent or antimicrobial agent) that is sufficient to effect treatment when administered to a mammal in need of such treatment, such as treatment of a cancer. The therapeutically effective amount will vary depending upon the subject and disease condition being treated, the weight and age of the subject, the severity of the disease condition, the manner of administration, and the like, which can readily be determined by a prescribing physician.

Treating or inhibiting a disease: Inhibiting the full development of a disease or condition, for example, in a subject who is at risk for a disease, such as a subject with cancer, for example, pancreatic cancer, or an infectious disease. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. Any success or indicia of success in the attenuation or amelioration of an injury, pathology, or condition, including any objective or subjective parameter such as abatement, remission, diminishing of symptoms or making the condition more tolerable to the patient, slowing in the rate of degeneration or decline, making the final point of degeneration less debilitating, improving a subject's sensorimotor function. The treatment may be assessed by objective or subjective parameters; including the results of a physical examination, neurological examination, or psychiatric evaluations. For example, treatment of a cancer can include decreasing the size, volume, or weight of a cancer, decrease the number, size, volume, or weight of metastases, or combinations thereof. A “prophylactic” treatment is a treatment administered to a subject who does not exhibit signs of a disease or exhibits only early signs for the purpose of decreasing the risk of developing pathology.

Tumor, neoplasia, malignancy, or cancer: A neoplasm is an abnormal growth of tissue or cells which results from excessive cell division. Neoplastic growth can produce a tumor. The amount of a tumor in an individual is the “tumor burden”, which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer. A “cancer” is a malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, cancer cells, for example pancreatic cells, are analyzed by the disclosed methods.

In one example, the caner analyzed, diagnosed, and/or treated with the disclosed methods is pancreatic cancer (such as neuroendocrine pancreatic cancer or exocrine pancreatic cancer, which includes adenocarcinoma (such as pancreatic ductal adenocarcinoma, PDA), squamous cell carcinoma, adenosquamous carcinoma, and colloid carcinoma).

Exemplary tumors, such as cancers, that can be analyzed, diagnosed, and/or treated with the disclosed methods include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T- cell malignant lymphoma). In one example, the tumor is an adenocarcinoma, such as a PDA.

The methods can also be used to analyze, diagnose, and/or treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia , and adult T-cell leukemia), lymphomas (such as Hodgkin's lymphoma and non-Hodgkin's lymphoma), and myelomas).

### Overview

The disclosed methods describe the first framework to analyze human somatic cell-microbiome interactions and tropism at the resolution of single cells in the tumor microenvironment. Its utility was shown herein through analyses of microbe-host cell tropism in PDA, which provided further evidence that the pancreas is not a sterile organ (Thomas & Jobin, Nat. Rev. Gastroenterol. Hepatol. 2020;17, 53-64). The findings made herein were validated by consistent observations in multiple cohorts, across three different technology platforms, and by reliable detection of known pancreatic microbes and the absence of common laboratory contaminants. This work identified a distinct and diverse pancreatic cancer microbiome and associated pancreatic dysbiosis with cell-type dependent cancer-related activities in the tumor microenvironment, including the complement cascade, DNA repair pathways, and Hippo signaling. Three tumor modalities (TS1: microbiome-poor, TS2: fungi-rich, TS3: bacteria-rich) were identified, each with distinct microbiome, genetic activities, and clinical attributes, providing evidence that intra-tumoral microorganisms influence the trajectory of tumor growth.

Without inferring causality from correlation, the observations herein contribute to the debate of tumor-microbiome hologenomic evolution, in which crosstalk amongst microbes and tumor, immune, and stromal cells can potentially modulate tumorigenesis and anti-tumor responses. Tumors of long-term survivors of pancreatic cancer produced neoantigens with homology to microbial peptides (Balachandran, et al., Nature. 2017;551, S12-S16). Unlike immunotherapy-responsive cancer-types, the majority of infiltrating lymphocytes in PDA were shown to be microbe-reactive, which may contribute to the lack of efficacy of immune checkpoint inhibitors (Feng, et al., Cancer Lett. 2017;407, 57-65). If PDA-infiltrating T-cells mostly display infection microenvironment reactions, then tumor neoantigens with homology to microbial peptides may increase susceptibility to anti-tumor immune responses. However, microbiota in the tumor microenvironment, or tumors expressing microbial antigens, may also contribute to the characteristic immunosuppression in PDA by attracting regulatory T-cells and then polarizing macrophages toward immunosuppressive phenotypes (Vitiello et al., Trends in Cancer. 2019;5, 670-676 and Pushalkar et al., Cancer Discov. 2018;8, 403-416). The relationship between neoantigens with microbial homology and anti-tumor responses may reflect a balance between the type of homology and neoantigen expression dynamics. Overall, observations described herein regarding these novel T-cell global transcriptomic reactions have implications for immunotherapy and cell therapy; differential therapeutic targeting of infection- or tumor-microenvironment reacting T-cells could improve clinical outcomes.

Finally, the signature of high intra-tumoral microbiome diversity by SAHMI predicted patients at risk of poor survival. This result was consistent across multiple cohorts and outperformed a leading predictor based on bulk shotgun sequencing data (Poore, et al., Nature. 202;579, 567-574), underscoring its clinical relevance. This finding is consistent with the argument that eliminating bacteria with antibiotics improves tumor responses to checkpoint inhibitors (Pushalkar, et al., Cancer Discov. 2018;8, 403-416), but contrasts with reports of increased intra-tumoral bacterial diversity in long-term survivors of pancreatic cancer (Riquelme, et al., Cell. 2019;178, 795-806.e12). This difference may be due to differences in technological platforms (bulk mRNA/single-cell mRNA/16S rRNA) and sample processing (fresh/frozen/formalin fixed paraffin embedded). Another possibility is that only a subset of the tumor-associated microbes promote tumor growth; as such higher overall diversity may suppress the effects of the pathogenic subset and confer a survival advantage.

The observations made herein at single cell resolution corroborate known tumor-microbiome associations identified using bulk genomic data, model systems, or targeted experiments (Vitiello, et al., Trends in Cancer. 2019;5, 670-676; Pushalkar, et al., Cancer Discov. 2018;8, 403-416; Aykut, et al., Nature. 2019;574, 264-267; Sethi, et al., Gastroenterology. 2019; 156, 2097-2115.e2; Poore, et al., Nature 2020;579, 567-574; Nejman, et al., Science. 2020;980, 973-980), and also identify new associations consistent across datasets. SAHMI creates opportunities to examine patterns of human-microbiome interactions from single-cell sequencing data without the need for additional experimental modifications, generating testable hypotheses about host-microbiome tropism at multiple levels. This framework is not tumor-specific and can be applied to study a variety of tissues and disease states, as well as other microscopic agents such as viruses or helminths.

### Methods

### Methods of Diagnosing and Prognosing Cancer in a Subject

The present disclosure provides methods for diagnosing and prognosing (e.g., predicting survival outcome) in a subject with cancer, for example by analyzing expression of microbial nucleic acid molecules in individual cells (e.g., single cells), such as individual cancer cells and corresponding normal cells (e.g., pancreatic cancer cells and normal pancreatic cells from the same subject), and in some examples individual microbial cells (e.g., individual bacterial cells and/or individual fungal cells). The nucleic acid sequences obtained from each individual cell (e.g., each single/individual cell in a larger population of cells), can be compared to a nucleic acid sequence database, such as a database that includes microbial nucleic acid sequences (such as bacterial nucleic acid sequences and/or fungal nucleic acid sequences). In some examples, the database includes bacterial nucleic acid sequences, parasitic nucleic acid sequences, viral nucleic acid sequences, and/or fungal nucleic acid sequences. In some examples, the nucleic acid sequences are RNA sequences. In some examples, the nucleic acid sequences are DNA sequences.

Analysis of nucleic acid sequences at the individual cell level allows for robust diagnosis and prognosis of cancer, such as pancreatic cancer, based on the presence of particular microbes associated with individual cells analyzed from tumor tissue, wherein microbe abundances are increased or decreased relative to a control (such as normal tissue of the same cell type). In one example, the presence of particular microbes in higher amounts in the tumor or tumor cells (e.g., pancreatic cancer cells), such as an increase in Prevotella, Megamonas, Spiroplasma, Bacteroides Polaribacter Arcobacter Acinetobacter Clostridium Chryseobacterium Lactobacillus Paenibacillus Flavobacterium Vibrio Mycoplasma Campylobacter Streptococcus Fusobacterium Buchnera Streptomyces Bacillus Kluyveromyces Sphingobacterium Saccharomyces Thermothielavioides Colletotrichum, and/or Aspergillus nucleic acid molecules relative to a control (such as normal tissue of the same cell type, such as normal pancreas tissue), can indicate the presence of cancer and/or a poor survival outcome. In one example, the presence of particular microbes in lower amounts in the tumor cells (e.g., pancreatic cancer cells), such as a decrease in abundance or no detection of Prevotella, Megamonas, Spiroplasma, Bacteroides Polaribacter Arcobacter Acinetobacter Clostridium Chryseobacterium Lactobacillus Paenibacillus Flavobacterium Vibrio Mycoplasma Campylobacter Streptococcus Fusobacterium Buchnera Streptomyces Bacillus Kluyveromyces Sphingobacterium Saccharomyces Thermothielavioides Colletotrichum, and/or Aspergillus nucleic acid molecules relative to a control (such as normal tissue of the same cell type, such as normal pancreatic tissue), can indicate the absence of cancer and/or a good outcome. In some examples, a poor survival outcome corresponds to a median survival of less than 800 days, less than 700 days, less than 650 days, or less than 603 days and increased microbial diversity in a sample from the subject. In some examples, a good survival outcome corresponds to a median survival of at least 1000 days, at least 1100 days, at least 1200 days, at least 1300 days, at least 1400 days, or at least 1502 days and reduced microbial diversity in a sample from the subject.

In one example, the presence of particular microbes in lower amounts in the tumor cells (e.g., pancreatic cancer cells), such as a decrease in Staphylococcus, Paraccocus, Burkholderia, Klebsiella, Pasteurella, and Ralstonia nucleic acid molecules relative to a control (such as normal tissue of the same cell type, such as normal pancreatic tissue), indicates the presence of cancer, or indicates a poor survival outcome in a subject with cancer (such as pancreatic cancer).

Based on the diagnosis or prognosis obtained, the subject can be treated appropriately, for example with an antimicrobial agent (such as one or more anti-fungal and/or one or more antibiotics) if increased Prevotella, Megamonas, Spiroplasma, Bacteroides Polaribacter Arcobacter Acinetobacter Clostridium Chryseobacterium Lactobacillus Paenibacillus Flavobacterium Vibrio Mycoplasma Campylobacter Streptococcus Fusobacterium Buchnera Streptomyces Bacillus Kluyveromyces Sphingobacterium Saccharomyces Thermothielavioides Colletotrichum, and/or Aspergillus nucleic acid molecules relative to a control (such as normal tissue of the same cell type, such as normal pancreatic tissue) are detected, and/or increased Staphylococcus, Paraccocus, Burkholderia, Klebsiella, Pasteurella, and Ralstonia nucleic acid molecules in normal tissue of the same cell type, such as normal pancreatic tissue, relative to individual cells obtained from the cancerous tissue (e.g., pancreatic cancer tissue) are detected. In some examples, such a subject is treated with one or more of surgery, radiation therapy, chemotherapy, a biologic (e.g., therapeutic monoclonal antibody), selective bacteriophage, and palliative care.

In some examples, treatment can decrease the size of a tumor (such as the volume or weight of a tumor or metastasis of a tumor), for example by at least 20%, at least 50%, at least 80%, at least 90%, at least 95%, at least 98%, or even substantially 100%, as compared to the tumor size in the absence of the treatment. In one particular example, treatment kills a population of cells (such as cancer cells), for example by killing at least 20%, at least 50%, at least 60%, at least 70%, at least 80%, at least 90%, at least 95%, at least 98%, or even substantially 100% of the cells, as compared to the cell killing in the absence of the treatment. In one particular example, treatment increases the survival time of a patient (such as increased progression-free survival time of the subject or increased disease-free survival time of the subject) by at least 20%, at least 50%, at least 60%, at least 70%, at least 80%, at least 90%, at least 95%, at least 98%, at least 100%, at least 200%, or at least 500%, as compared to the survival time in the absence of the treatment. In some examples, the survival time of a subject increases by at least 2 months, at least 3 months, at least 4 months, at least 5 months, at least 6 months, at least 9 months, at least 1 year, at least 1.5 years, at least 2 years, at least 3 years, at least 4 years, at least 5 years or more, for example relative to the absence of treatment. In some examples, treatment increases a subject's progression-free survival time or disease-free survival time (for example, lack of recurrence of the primary tumor or lack of metastasis) by at least 1 months, at least 2 months, at least 3 months, at least 6 months, at least 12 months, at least 18 months, at least 24 months, at least 36 months, at least 48 months, at least 60 months, or more, relative to average survival time in the absence of treatment.

In some embodiments, cancer detection is achieved by comparing expression data (such as gene expression information) from the subject to a control. In some embodiments, gene expression is analyzed using one or more methods disclosed herein, such as RNA-sequencing (RNA-seq), such as single cell RNA-sequencing (scRNA-seq). In certain embodiments, expression data from the subject can include human gene expression information or non-human gene expression information, or a combination thereof. Non-human expression information from the subject, such as expression data obtained using RNA-seq (such as scRNA-seq), can include microbial gene expression information, such as bacterial and/or fungal gene expression information. In the disclosed methods, gene expression data from a subject may be analyzed to detect the presence of absence of one or more bacteria and/or fungi, for example, of genera Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia.

The methods provided herein can further include detecting expression (such as gene expression) of molecules, such as cancer-related molecules, in cancer samples (such as pancreatic cancer samples) and/or control samples (such as non-cancerous samples from the same tissue type, such as normal non-cancerous pancreatic tissue samples). In some embodiments, the methods include detection of one or more, such as 1-10, housekeeping genes.

In some embodiments, expression levels of a set of six genes (the six-gene signature) is used to classify the subject as having a poor or good survival outcome. The six-gene signature can be used to classify the sample as having low or high microbial diversity. In specific embodiments, the genes of the six-gene signature are nth like DNA glycosylase 1 (NTHL1; e.g., GENBANK® Accession No. U81285.1), ly6/PLAUR domain-containing protein 2 (LYPD2; e.g., GENBANK® Accession No. AY358432.1), mucin-16 (MUC16; e.g., GENBANK® Accession No. AF414442.2), C2 calcium-dependent domain-containing protein 4B (C2CD4B; e.g., GENBANK® Accession No. BM023530.1), flavin containing dimethylaniline monooxygenase 3 (FMO3; e.g., GENBANK® Accession No. BC032016.1), and interleukin-1 receptor-like 1 (IL1RL1; e.g., GENBANK® Accession No. AB012701.3). In other specific embodiments, increased expression of one or more of IL1RL1, C2CD4B, FMO3, or NTHL1 compared to a control, and/or decreased expression of one or more of LYPD2 or MUC16 compared to the control indicates high microbial diversity in the subject and classifies the subject as having a poor survival outcome. In yet another specific embodiment, decreased expression of one or more of IL1RL1, C2CD4B, FMO3, or NTHL1 compared to a control, and/or increased expression of one or more of LYPD2 or MUC16 compared to the control indicates low microbial diversity in the subject and classifies the subject as having a good survival outcome. In some embodiments, classifying the subject as having a poor or good survival outcome comprises calculating the Shannon diversity index for the sample based on its profiled microbiome compared to a control, thereby determining the microbial diversity of the sample. In another embodiment, classifying the subject as having a poor or good survival outcome comprises using the ranked expression levels of the set of six genes in the sample and the associated random forest model to predict diversity and survival. The control can be any control sample as disclosed herein. In one example the control is individual non-cancerous/normal cells of the same tissue type, or values (or a range of values) that represents expression for each of NTHL1, LYPD2, MUC16, C2CD4B, FMO3, and IL1RL1 in such cells.

For example, expression of NTHL1, LYPD2, MUC16, C2CD4B, FMO3, and IL1RL1 nucleic acid molecules in a tumor sample is determined. In some examples, expression levels of these six molecules are quantified. Expression of nucleic acid sequences obtained from the individual cancer cells can be compared to a nucleic acid expression in non-cancerous/normal cells of the same tissue type.

### Methods of Determining T-Cell Microenvironment Reaction in a Subject

Also disclosed are methods of determining T-cell microenvironment reaction in a subject. T-cells, which can be identified using biological markers known to one of ordinary skill in the art, can be classified as described herein (Examples 1 and 2) as displaying a transcriptional phenotype classified as having either a tumor microenvironment reaction (TMER) or infection microenvironment reaction (IMER). As described herein, in many tumors where immunotherapies are efficacious, and where the microbiome burden is also low, T-cells isolated from tumor samples were classified primarily as TMER. Conversely, in pancreatic cancer where immunotherapies are typically not effective and where the microbiome burden appears higher, T-cells isolated from tumor samples were primarily classified as IMER. Knowledge of the T-cell microenvironment reaction status of a subject may allow for administration of therapies that specifically activate tumor reactive T-cells to target a tumor in the subject. Similarly, specific T-cells could be selected for when developing autologous cell therapies such as CAR-T-cell therapy.

Classification of T-cells isolated from a subject as TMER or IMER can be accomplished by sequencing (such as by scRNA-seq) nucleic acids collected from the T-cells. Expression levels (such as determined using scRNA-seq analysis) of a set of genes in individual T-cells from the subject can be compared to expression levels of a pre-selected set of genes, wherein differences in expression levels of one or more of the genes in the individual T-cells as compared to expression levels of the one or more genes as determined by a model can indicate whether an individual T-cell is IMER or TMER. For example, a model can be trained to classify T-cells as either IMER or TMER using gene expression data for T-cells isolated from subjects having an infection, such as sepsis, and from subjects having a cancer, such as a cancer having lung cancer or pancreatic cancer (Examples 1 and 2). In some examples, the set of genes comprises the genes of Table 2. In a specific example, the set of genes consists of the set of genes of Table 2.

In some embodiments, expression levels of a set of one or more genes in Table 2 (such as at least two, at least three, at least 4, at least 5, at least 10, at least 20, at least 50, at least 100, at least 200, at least 300, at least 400, at least 500, or all of the genes in Table 2) can be measured in isolated T cells (such as a T cells from or near a tumor, such as pancreatic cancer) to determine the reactivity of the T cells. In some examples, such a method further includes treating a patient diagnosed with cancer, such as treatment with one or more of surgery, radiation therapy, chemotherapy, antimicrobial (e.g., antifungal and/or antibiotic), biologic, selective bacteriophage, and palliative care.

TABLE 2
Exemplary genes (T-cell microenvironment reaction signature,
Examples 1 and 2) used to classify T-cells isolated from a subject
as tumor-reactive or microbe-reactive. “Mean decrease accuracy”
for a gene indicates the change in model classification accuracy
when the value of the gene is randomly permuted.

| | Gene | Mean Decrease Accuracy |
|---|---|---|
| 1 | S100A8 | 0.092773561 |
| 2 | RPL41 | 0.078648903 |
| 3 | RPL39 | 0.039672861 |
| 4 | S100A9 | 0.028971284 |
| 5 | RPS27 | 0.009858452 |
| 6 | RPS29 | 0.00877185 |
| 7 | NKG7 | 0.008558657 |
| 8 | TYROBP | 0.007349671 |
| 9 | RPS28 | 0.006257825 |
| 10 | LYZ | 0.005155002 |
| 11 | RPS26 | 0.004307184 |
| 12 | S100A12 | 0.003465595 |
| 13 | LST1 | 0.002760927 |
| 14 | GNLY | 0.002602244 |
| 15 | TMSB10 | 0.002425835 |
| 16 | RPL13A | 0.002377481 |
| 17 | EEF1A1 | 0.002302028 |
| 18 | FCN1 | 0.002029348 |
| 19 | MYL6 | 0.001801459 |
| 20 | PLEKHJ1 | 0.00170777 |
| 21 | CLTB | 0.001534479 |
| 22 | RPL24 | 0.001495799 |
| 23 | ST13 | 0.001426953 |
| 24 | RGS19 | 0.001284512 |
| 25 | RPL36A | 0.001254065 |
| 26 | RPS7 | 0.001246853 |
| 27 | DNAJC7 | 0.001213409 |
| 28 | GRN | 0.001194567 |
| 29 | ATP5G3 | 0.001172354 |
| 30 | CANX | 0.001162368 |
| 31 | C1orf56 | 0.001147811 |
| 32 | H3F3A | 0.001121218 |
| 33 | KLRD1 | 0.001087927 |
| 34 | RPL13 | 0.001066884 |
| 35 | PAK2 | 0.001064609 |
| 36 | FRG1 | 0.001055478 |
| 37 | TMEM256 | 0.001021827 |
| 38 | RPS9 | 0.000996953 |
| 39 | LPAR6 | 0.000961476 |
| 40 | BCLAF1 | 0.000931859 |
| 41 | RPS16 | 0.000921339 |
| 42 | MIEN1 | 0.000908645 |
| 43 | TMEM179B | 0.000891395 |
| 44 | SNHG9 | 0.000876477 |
| 45 | STAT1 | 0.000855168 |
| 46 | ATP5G2 | 0.000842925 |
| 47 | RPS4X | 0.000839862 |
| 48 | S100A11 | 0.000834713 |
| 49 | RPL15 | 0.000830827 |
| 50 | AHNAK | 0.000826019 |
| 51 | SMS | 0.000824325 |
| 52 | COX4I1 | 0.000822374 |
| 53 | HMHA1 | 0.000816084 |
| 54 | HSBP1 | 0.000812709 |
| 55 | YIPF4 | 0.000803081 |
| 56 | RPL29 | 0.000801736 |
| 57 | LCP1 | 0.000801253 |
| 58 | SNRPE | 0.000774927 |
| 59 | SVIP | 0.000771892 |
| 60 | RPL19 | 0.000764541 |
| 61 | FCER1G | 0.000744006 |
| 62 | CAPZA2 | 0.0007394 |
| 63 | CFL1 | 0.000732052 |
| 64 | EDF1 | 0.000720073 |
| 65 | VCAN | 0.000719759 |
| 66 | SDF2L1 | 0.000718715 |
| 67 | KRTCAP2 | 0.000713555 |
| 68 | CBX3 | 0.000713553 |
| 69 | NUCKS1 | 0.000702455 |
| 70 | RPL14 | 0.000702164 |
| 71 | DNAJC19 | 0.000695716 |
| 72 | RPLP1 | 0.000694564 |
| 73 | PGAM1 | 0.000689222 |
| 74 | C5orf56 | 0.00068649 |
| 75 | SPCS3 | 0.000685822 |
| 76 | MBP | 0.000676305 |
| 77 | HNRNPH1 | 0.000671656 |
| 78 | POLR2K | 0.00066548 |
| 79 | GNAI2 | 0.000656285 |
| 80 | SRRM2 | 0.00065613 |
| 81 | ZNHIT1 | 0.000654315 |
| 82 | SUB1 | 0.000644202 |
| 83 | LITAF | 0.000625774 |
| 84 | RPL36AL | 0.000625117 |
| 85 | CRIP1 | 0.000621146 |
| 86 | NDUFB11 | 0.000617543 |
| 87 | MOB1A | 0.000607107 |
| 88 | NDUFB4 | 0.000601115 |
| 89 | CST3 | 0.000595673 |
| 90 | SUMO2 | 0.000594374 |
| 91 | SRSF5 | 0.000593552 |
| 92 | NHP2 | 0.000584724 |
| 93 | HINT1 | 0.000583941 |
| 94 | LTB | 0.000574929 |
| 95 | CALM2 | 0.000564717 |
| 96 | EIF4B | 0.000564267 |
| 97 | COX20 | 0.000564044 |
| 98 | ARL5A | 0.000558315 |
| 99 | SYTL1 | 0.000553772 |
| 100 | PGLS | 0.000552433 |
| 101 | AIF1 | 0.000536204 |
| 102 | FGFBP2 | 0.000518878 |
| 103 | PRDM1 | 0.000513088 |
| 104 | UXT | 0.000511949 |
| 105 | C9orf16 | 0.000510293 |
| 106 | SNRPF | 0.00050393 |
| 107 | GZMH | 0.000501027 |
| 108 | POLR2F | 0.000498148 |
| 109 | NBEAL1 | 0.000494553 |
| 110 | SPN | 0.000492723 |
| 111 | TOMM7 | 0.000492541 |
| 112 | GABARAP | 0.000491839 |
| 113 | C17orf89 | 0.000488652 |
| 114 | GNB2 | 0.00048578 |
| 115 | CTSS | 0.000483926 |
| 116 | IFITM2 | 0.000483421 |
| 117 | CHCHD10 | 0.00047783 |
| 118 | VPS29 | 0.00047611 |
| 119 | JTB | 0.000471909 |
| 120 | APRT | 0.00046291 |
| 121 | RPL23A | 0.000460485 |
| 122 | CUTA | 0.000455038 |
| 123 | PTPN4 | 0.000454714 |
| 124 | OXLD1 | 0.000454202 |
| 125 | UBE2D1 | 0.000450914 |
| 126 | CYBB | 0.000447317 |
| 127 | RPS17 | 0.000442033 |
| 128 | PTMA | 0.000435696 |
| 129 | CD164 | 0.00043541 |
| 130 | C19orf70 | 0.000434591 |
| 131 | TSC22D4 | 0.000434491 |
| 132 | PSIP1 | 0.00042833 |
| 133 | PAN3 | 0.000423481 |
| 134 | TRMT112 | 0.000422168 |
| 135 | RPS3A | 0.00042108 |
| 136 | SLC9A3R1 | 0.000420697 |
| 137 | TCEA1 | 0.000420685 |
| 138 | FGR | 0.000418293 |
| 139 | HNRNPU | 0.000417556 |
| 140 | NDUFB3 | 0.000415965 |
| 141 | GPX4 | 0.000415181 |
| 142 | CHCHD5 | 0.000411257 |
| 143 | TES | 0.000410229 |
| 144 | ANAPC16 | 0.00040612 |
| 145 | DDX18 | 0.000405842 |
| 146 | FAU | 0.000401403 |
| 147 | ZC3HAV1 | 0.000384626 |
| 148 | HLA.DRA | 0.000383825 |
| 149 | BIN2 | 0.000382106 |
| 150 | DDX17 | 0.000375848 |
| 151 | HP1BP3 | 0.000373013 |
| 152 | PTPRC | 0.000367906 |
| 153 | RPL17 | 0.000365804 |
| 154 | PPIA | 0.000364396 |
| 155 | CCL5 | 0.000357919 |
| 156 | COX6A1 | 0.00035501 |
| 157 | LSM7 | 0.000352817 |
| 158 | RPL23 | 0.000341939 |
| 159 | STT3B | 0.000340606 |
| 160 | ZNF428 | 0.000339031 |
| 161 | VAMP8 | 0.000338092 |
| 162 | RPL6 | 0.000337001 |
| 163 | CD8A | 0.000334106 |
| 164 | POLR2I | 0.000333499 |
| 165 | ARHGAP30 | 0.000332356 |
| 166 | TTC14 | 0.000332236 |
| 167 | RPS18 | 0.000331036 |
| 168 | LSM6 | 0.000328714 |
| 169 | SSR4 | 0.00032843 |
| 170 | CLEC2B | 0.000324736 |
| 171 | GPSM3 | 0.000324493 |
| 172 | SRSF9 | 0.00032395 |
| 173 | PNRC1 | 0.000323715 |
| 174 | DUSP2 | 0.00032276 |
| 175 | LRRFIP1 | 0.000321934 |
| 176 | RNF213 | 0.000321411 |
| 177 | ERH | 0.000321181 |
| 178 | COX7A2 | 0.000321011 |
| 179 | NAA10 | 0.000317172 |
| 180 | PA2G4 | 0.000315746 |
| 181 | CDC42SE1 | 0.000313487 |
| 182 | NDUFB2 | 0.000311815 |
| 183 | FAM195B | 0.000311799 |
| 184 | NDUFB9 | 0.000311013 |
| 185 | RPL11 | 0.000304608 |
| 186 | JOSD2 | 0.000301649 |
| 187 | HMGN2 | 0.000298708 |
| 188 | SFPQ | 0.000294578 |
| 189 | BANF1 | 0.000292952 |
| 190 | ZNF207 | 0.000292714 |
| 191 | CHURC1 | 0.000292499 |
| 192 | SNX3 | 0.000289765 |
| 193 | NENF | 0.000287824 |
| 194 | C16orf13 | 0.000282382 |
| 195 | CKLF | 0.00028194 |
| 196 | CISD3 | 0.000281576 |
| 197 | RHOF | 0.000280805 |
| 198 | POLE4 | 0.000279025 |
| 199 | RPS5 | 0.00027819 |
| 200 | MYO1G | 0.00027809 |
| 201 | NDUFA1 | 0.000272964 |
| 202 | NOSIP | 0.00026912 |
| 203 | PDCD5 | 0.000266742 |
| 204 | EMP3 | 0.000266521 |
| 205 | SUN2 | 0.000263091 |
| 206 | AURKAIP1 | 0.000256714 |
| 207 | IKZF1 | 0.000255782 |
| 208 | UBXN11 | 0.000254844 |
| 209 | HMGN1 | 0.00025374 |
| 210 | MINOS1 | 0.000252667 |
| 211 | ABHD17A | 0.000251988 |
| 212 | RNASEH2C | 0.000251803 |
| 213 | C14orf2 | 0.000250531 |
| 214 | RASGRP2 | 0.000249522 |
| 215 | FMNL1 | 0.000247154 |
| 216 | CDKN2D | 0.000247119 |
| 217 | MTPN | 0.000246429 |
| 218 | TBCA | 0.00024378 |
| 219 | TTC19 | 0.000242335 |
| 220 | RPL36 | 0.000241997 |
| 221 | RPS13 | 0.000240079 |
| 222 | ATP5L | 0.000235236 |
| 223 | ANXA2R | 0.000233451 |
| 224 | ATOX1 | 0.000233108 |
| 225 | EIF4E | 0.000230816 |
| 226 | C7orf73 | 0.000229408 |
| 227 | TMC6 | 0.000228813 |
| 228 | TCF25 | 0.000225841 |
| 229 | DNAJB11 | 0.000225338 |
| 230 | TMEM219 | 0.000225184 |
| 231 | OAZ1 | 0.000220815 |
| 232 | RPS8 | 0.000220254 |
| 233 | CTSW | 0.000219513 |
| 234 | RPL38 | 0.000219489 |
| 235 | CBX6 | 0.000219195 |
| 236 | ATP5D | 0.000218966 |
| 237 | SPI1 | 0.000218858 |
| 238 | SEC61B | 0.000218251 |
| 239 | LINC00861 | 0.0002166 |
| 240 | CAPZA1 | 0.000216269 |
| 241 | MDM4 | 0.000215343 |
| 242 | ANKRD44 | 0.00021133 |
| 243 | LAMTOR4 | 0.000211294 |
| 244 | SRP9 | 0.000208176 |
| 245 | C19orf60 | 0.000207567 |
| 246 | OST4 | 0.000204408 |
| 247 | PTPN6 | 0.000202001 |
| 248 | LY6E | 0.000199901 |
| 249 | RPS21 | 0.000198975 |
| 250 | PSMB9 | 0.000198929 |
| 251 | NDUFB10 | 0.000198852 |
| 252 | ZEB2 | 0.000198632 |
| 253 | POLD4 | 0.000198133 |
| 254 | MIF | 0.000196685 |
| 255 | RTF1 | 0.000196359 |
| 256 | CLIC3 | 0.00019608 |
| 257 | RPS10 | 0.00019481 |
| 258 | PABPN1 | 0.000190371 |
| 259 | NOP10 | 0.000187697 |
| 260 | CNN2 | 0.000186634 |
| 261 | DSTN | 0.0001864 |
| 262 | SNF8 | 0.000184977 |
| 263 | LYAR | 0.000184208 |
| 264 | ZNF302 | 0.00018386 |
| 265 | COX6B1 | 0.000181034 |
| 266 | HNRNPC | 0.000179594 |
| 267 | WDR83OS | 0.000179507 |
| 268 | CMC1 | 0.000179313 |
| 269 | PIM1 | 0.000177959 |
| 270 | MBNL1 | 0.000177547 |
| 271 | RBL2 | 0.000177351 |
| 272 | GLIPR2 | 0.000177274 |
| 273 | PFN1 | 0.000176772 |
| 274 | POLR2J3 | 0.000175978 |
| 275 | TMEM167A | 0.000174243 |
| 276 | TGFB1 | 0.000173874 |
| 277 | IFITM1 | 0.000172206 |
| 278 | SNRPD2 | 0.000171796 |
| 279 | PRELID1 | 0.000171214 |
| 280 | RPL34 | 0.000170164 |
| 281 | PCNP | 0.000169875 |
| 282 | CDC42 | 0.000169503 |
| 283 | SSU72 | 0.000168608 |
| 284 | PTEN | 0.000166418 |
| 285 | ZFAS1 | 0.000165881 |
| 286 | UQCRH | 0.000164478 |
| 287 | C16orf54 | 0.000164119 |
| 288 | COX17 | 0.000160223 |
| 289 | ANAPC11 | 0.000156723 |
| 290 | CSK | 0.000156271 |
| 291 | FCGRT | 0.000155045 |
| 292 | RPL27 | 0.00015459 |
| 293 | LAMTOR2 | 0.000154483 |
| 294 | KRT10 | 0.000151949 |
| 295 | ARL6IP4 | 0.000151258 |
| 296 | IFI27L2 | 0.00014985 |
| 297 | ROMO1 | 0.000148865 |
| 298 | RPL28 | 0.000147802 |
| 299 | RNF167 | 0.000146421 |
| 300 | RPL30 | 0.000144795 |
| 301 | EIF5B | 0.000143641 |
| 302 | NCL | 0.000143211 |
| 303 | MMP24.AS1 | 0.000142412 |
| 304 | NDUFA13 | 0.000142261 |
| 305 | CFD | 0.000138063 |
| 306 | ATP5I | 0.000137571 |
| 307 | LINC00116 | 0.000136984 |
| 308 | TRAPPC1 | 0.000135245 |
| 309 | TSPO | 0.000133668 |
| 310 | DRAP1 | 0.000133384 |
| 311 | RPL27A | 0.000132097 |
| 312 | RAP1B | 0.000131245 |
| 313 | RPL12 | 0.000131086 |
| 314 | CAST | 0.000131013 |
| 315 | COMMD6 | 0.000128804 |
| 316 | CD14 | 0.000128137 |
| 317 | CNPY3 | 0.000126885 |
| 318 | RPS23 | 0.000126683 |
| 319 | COX7C | 0.000126265 |
| 320 | C11orf31 | 0.000126193 |
| 321 | TCEB2 | 0.000124652 |
| 322 | N4BP2L2 | 0.000124328 |
| 323 | TXNL4A | 0.000123254 |
| 324 | RPLP2 | 0.000122565 |
| 325 | FTL | 0.000122391 |
| 326 | HMGN3 | 0.00012163 |
| 327 | C19orf53 | 0.000119653 |
| 328 | TMA7 | 0.000119204 |
| 329 | PTP4A2 | 0.000118152 |
| 330 | ZRANB2 | 0.000117696 |
| 331 | COX7B | 0.000115701 |
| 332 | COX8A | 0.000115313 |
| 333 | VAMP2 | 0.000112998 |
| 334 | CST7 | 0.000112812 |
| 335 | MRPS21 | 0.00011245 |
| 336 | PPP3CA | 0.000111714 |
| 337 | DAZAP2 | 0.000110912 |
| 338 | LSM4 | 0.000110902 |
| 339 | DBI | 0.000110782 |
| 340 | TRA2B | 0.000109346 |
| 341 | NDUFA4 | 0.000109301 |
| 342 | TAOK3 | 0.000108586 |
| 343 | ATP5G1 | 0.000108582 |
| 344 | EFHD2 | 0.000106692 |
| 345 | FAM107B | 0.000106359 |
| 346 | FAM133B | 0.000104905 |
| 347 | ARPC5 | 0.000103902 |
| 348 | PYHIN1 | 0.000102734 |
| 349 | DOK2 | 0.00010235 |
| 350 | RPL22 | 0.000101582 |
| 351 | MRPL41 | 9.94E−05 |
| 352 | FLT3LG | 9.86E−05 |
| 353 | UBA52 | 9.81E−05 |
| 354 | PFDN5 | 9.78E−05 |
| 355 | TRAM1 | 9.76E−05 |
| 356 | POLR2J | 9.63E−05 |
| 357 | TOPORS.AS1 | 9.52E−05 |
| 358 | FIS1 | 9.50E−05 |
| 359 | PCBP1 | 9.50E−05 |
| 360 | TIMM13 | 9.11E−05 |
| 361 | SNRPG | 9.03E−05 |
| 362 | BRI3 | 9.00E−05 |
| 363 | ATP5J | 8.91E−05 |
| 364 | STK17B | 8.90E−05 |
| 365 | RPS15 | 8.87E−05 |
| 366 | BEST1 | 8.66E−05 |
| 367 | JAK1 | 8.66E−05 |
| 368 | RPS25 | 8.64E−05 |
| 369 | NDUFA2 | 8.38E−05 |
| 370 | CLEC2D | 8.18E−05 |
| 371 | FOXP1 | 8.16E−05 |
| 372 | STUB1 | 8.13E−05 |
| 373 | AAK1 | 7.98E−05 |
| 374 | SPON2 | 7.95E−05 |
| 375 | MRPL33 | 7.92E−05 |
| 376 | RPL21 | 7.92E−05 |
| 377 | SET | 7.89E−05 |
| 378 | POMP | 7.66E−05 |
| 379 | LSM5 | 7.51E−05 |
| 380 | KLF2 | 7.50E−05 |
| 381 | TMED2 | 7.40E−05 |
| 382 | TRAF3IP3 | 7.37E−05 |
| 383 | SRSF3 | 7.35E−05 |
| 384 | C19orf24 | 7.33E−05 |
| 385 | GPR65 | 7.32E−05 |
| 386 | PPDPF | 7.16E−05 |
| 387 | PRR13 | 7.15E−05 |
| 388 | COX5B | 7.13E−05 |
| 389 | ATP5E | 7.12E−05 |
| 390 | COTL1 | 7.09E−05 |
| 391 | RPS27A | 7.05E−05 |
| 392 | B3GAT2 | 6.84E−05 |
| 393 | ATP5EP2 | 6.80E−05 |
| 394 | CNOT7 | 6.79E−05 |
| 395 | SEPW1 | 6.62E−05 |
| 396 | H1FX | 6.59E−05 |
| 397 | PRPF4B | 6.56E−05 |
| 398 | GZMA | 6.53E−05 |
| 399 | SF1 | 6.44E−05 |
| 400 | COX6C | 6.29E−05 |
| 401 | PSAP | 6.28E−05 |
| 402 | ATP5J2 | 6.26E−05 |
| 403 | RPS19 | 6.26E−05 |
| 404 | CCDC85B | 6.24E−05 |
| 405 | GRK6 | 6.23E−05 |
| 406 | CD3G | 6.22E−05 |
| 407 | MYO1F | 6.21E−05 |
| 408 | GUK1 | 6.16E−05 |
| 409 | CD8B | 6.06E−05 |
| 410 | TRA2A | 6.05E−05 |
| 411 | SAMD3 | 6.03E−05 |
| 412 | IRF1 | 6.02E−05 |
| 413 | ATM | 5.99E−05 |
| 414 | LGALS1 | 5.98E−05 |
| 415 | PRF1 | 5.70E−05 |
| 416 | BCL11B | 5.69E−05 |
| 417 | RPL37A | 5.68E−05 |
| 418 | IL16 | 5.62E−05 |
| 419 | SUMO1 | 5.46E−05 |
| 420 | HCST | 5.45E−05 |
| 421 | TMSB4X | 5.43E−05 |
| 422 | YPEL3 | 5.20E−05 |
| 423 | PRDX5 | 5.20E−05 |
| 424 | RPS14 | 5.19E−05 |
| 425 | RPL35A | 5.10E−05 |
| 426 | CD47 | 4.89E−05 |
| 427 | NDUFA11 | 4.88E−05 |
| 428 | PNISR | 4.77E−05 |
| 429 | RPL32 | 4.65E−05 |
| 430 | SRM | 4.65E−05 |
| 431 | ETS1 | 4.62E−05 |
| 432 | CD52 | 4.61E−05 |
| 433 | SRRM1 | 4.57E−05 |
| 434 | NAA38 | 4.57E−05 |
| 435 | UQCR10 | 4.52E−05 |
| 436 | PCBP2 | 4.46E−05 |
| 437 | SH3BGRL3 | 4.40E−05 |
| 438 | MZT2B | 4.39E−05 |
| 439 | SSBP4 | 4.38E−05 |
| 440 | AGTRAP | 4.36E−05 |
| 441 | PYCARD | 4.30E−05 |
| 442 | PPP1CB | 4.27E−05 |
| 443 | S100A6 | 4.19E−05 |
| 444 | APOBEC3C | 4.14E−05 |
| 445 | NDUFS6 | 4.13E−05 |
| 446 | ARF6 | 4.10E−05 |
| 447 | ZYX | 4.09E−05 |
| 448 | SLIRP | 4.08E−05 |
| 449 | UBL5 | 4.06E−05 |
| 450 | RBX1 | 4.05E−05 |
| 451 | KLRG1 | 3.86E−05 |
| 452 | RPS15A | 3.85E−05 |
| 453 | AES | 3.84E−05 |
| 454 | CTNNB1 | 3.80E−05 |
| 455 | FUS | 3.76E−05 |
| 456 | BAX | 3.74E−05 |
| 457 | RSL24D1 | 3.58E−05 |
| 458 | RBBP4 | 3.54E−05 |
| 459 | CMPK1 | 3.52E−05 |
| 460 | TBC1D10C | 3.49E−05 |
| 461 | RPL31 | 3.47E−05 |
| 462 | PSME2 | 3.34E−05 |
| 463 | TNRC6B | 3.29E−05 |
| 464 | NEDD8 | 3.28E−05 |
| 465 | MYEOV2 | 3.28E−05 |
| 466 | RPL18A | 3.25E−05 |
| 467 | SCAF11 | 3.23E−05 |
| 468 | ITGB1 | 3.19E−05 |
| 469 | MT2A | 3.05E−05 |
| 470 | SEC62 | 2.99E−05 |
| 471 | RPS27L | 2.99E−05 |
| 472 | EIF5A | 2.98E−05 |
| 473 | RPL35 | 2.98E−05 |
| 474 | C6orf62 | 2.97E−05 |
| 475 | CDC42SE2 | 2.75E−05 |
| 476 | EPC1 | 2.69E−05 |
| 477 | GZMM | 2.69E−05 |
| 478 | GNG5 | 2.67E−05 |
| 479 | HOPX | 2.48E−05 |
| 480 | ATP6V0B | 2.48E−05 |
| 481 | FLNA | 2.46E−05 |
| 482 | CSNK1A1 | 2.46E−05 |
| 483 | NDUFC1 | 2.41E−05 |
| 484 | RPS24 | 2.35E−05 |
| 485 | SERPINA1 | 2.34E−05 |
| 486 | SRSF6 | 2.30E−05 |
| 487 | ANP32E | 2.16E−05 |
| 488 | C1orf162 | 2.15E−05 |
| 489 | CYBA | 2.13E−05 |
| 490 | KLRB1 | 2.13E−05 |
| 491 | ARGLU1 | 2.07E−05 |
| 492 | PET100 | 1.99E−05 |
| 493 | RPL37 | 1.92E−05 |
| 494 | RPS12 | 1.91E−05 |
| 495 | MIB2 | 1.91E−05 |
| 496 | EIF2S3 | 1.90E−05 |
| 497 | AP2S1 | 1.89E−05 |
| 498 | GZMB | 1.65E−05 |
| 499 | FAM49B | 1.65E−05 |
| 500 | UQCRQ | 1.64E−05 |
| 501 | FKBP2 | 1.64E−05 |
| 502 | NDUFB1 | 1.64E−05 |
| 503 | CEBPD | 1.63E−05 |
| 504 | PRMT2 | 1.63E−05 |
| 505 | VAMP5 | 1.62E−05 |
| 506 | PLAC8 | 1.61E−05 |
| 507 | CCL4 | 1.61E−05 |
| 508 | EIF1AX | 1.57E−05 |
| 509 | EIF3E | 1.55E−05 |
| 510 | ARRDC3 | 1.49E−05 |
| 511 | KTN1 | 1.38E−05 |
| 512 | XIST | 1.38E−05 |
| 513 | RAC1 | 1.37E−05 |
| 514 | ITGB2 | 1.37E−05 |
| 515 | BLOC1S1 | 1.36E−05 |
| 516 | PYURF | 1.35E−05 |
| 517 | ADD3 | 1.34E−05 |
| 518 | ATPIF1 | 1.30E−05 |
| 519 | SMDT1 | 1.11E−05 |
| 520 | CARD16 | 1.10E−05 |
| 521 | DDX6 | 1.05E−05 |
| 522 | NCF1 | 1.04E−05 |
| 523 | SLC25A37 | 8.44E−06 |
| 524 | MRPL52 | 8.40E−06 |
| 525 | NDUFA3 | 8.16E−06 |
| 526 | SEC61G | 8.05E−06 |
| 527 | MGEA5 | 7.99E−06 |
| 528 | STAG2 | 7.94E−06 |
| 529 | S100A4 | 7.78E−06 |
| 530 | C12orf75 | 5.46E−06 |
| 531 | AP1S2 | 5.39E−06 |
| 532 | IFITM3 | 5.31E−06 |
| 533 | TYMP | 5.25E−06 |
| 534 | MRPL23 | 5.24E−06 |
| 535 | YWHAZ | 3.56E−06 |
| 536 | ACTR2 | 3.13E−06 |
| 537 | RPL26 | 2.89E−06 |
| 538 | POLR2L | 2.77E−06 |
| 539 | LIMD2 | 2.73E−06 |
| 540 | SERF2 | 2.71E−06 |
| 541 | CEBPB | 2.38E−06 |
| 542 | PIP4K2A | 2.30E−06 |
| 543 | SARIA | 4.90E−07 |
| 544 | TMEM160 | 1.82E−07 |
| 545 | STXBP2 | 2.10E−08 |
| 546 | USMG5 | −3.23E−08 |
| 547 | ARPC4 | −7.70E−07 |
| 548 | NDUFB7 | −2.66E−06 |
| 549 | C4orf48 | −2.74E−06 |
| 550 | FAM65B | −4.73E−06 |
| 551 | GPX1 | −6.26E−06 |
| 552 | WTAP | −7.70E−06 |
| 553 | TMEM258 | −8.27E−06 |
| 554 | C9orf142 | −1.38E−05 |
| 555 | ZNF90 | −1.43E−05 |
| 556 | GSTP1 | −1.68E−05 |

### Exemplary Samples

The disclosed methods can include obtaining a biological sample from the subject. A “sample” can refer to part of a tissue that is either the entire tissue, or a diseased or healthy portion of the tissue. The sample can include cells (such as mammalian and microbial cells) and associated includes nucleic acid molecules. Such samples include, but are not limited to, tissue from biopsies (including formalin-fixed paraffin-embedded tissue), autopsies, and pathology specimens; sections of tissues (such as frozen sections or paraffin-embedded sections taken for histological purposes); body fluids, such as blood, sputum, serum, ejaculate, or urine, or fractions of any of these; and so forth. In one example, the sample is a fine needle aspirate.

In one particular example, the sample from the subject is a tissue biopsy sample. In another specific example, the sample from the subject is a pancreatic tissue sample. In some examples, the sample includes T cells from the subject, such as a subject with cancer.

In several embodiments, the biological sample is from a subject suspected of having a cancer, such as pancreatic, stomach cancer, colon cancer, breast cancer, uterine cancer, bladder, head and neck, kidney, liver, ovarian, pancreas, prostate, kidney, or rectum cancer. In some embodiments, the biological sample is a tumor sample or a suspected tumor sample. For example, the sample can be a biopsy sample from at or near or just beyond the perceived leading edge of a tumor in a subject. Testing of the sample using the methods provided herein can be used to confirm the location of the leading edge of the tumor in the subject. This information can be used, for example, to determine if further surgical removal of tumor tissue is appropriate, and/or if certain treatments or treatment methods are appropriate for use in the subject.

In other embodiments, the biological sample is from a subject suspected of having an infection, such as a Candida albicans, human immunodeficiency virus (HIV), Helicobacter pylori, alphaherpesvirus, Mycobacterium leprae, Mycobacterium tuberculosis, Salmonella enterica, or a coronavirus (such as MERS or SARS, such as SARS-COV or SARS-COV-2) infection.

As described herein, samples obtained from a subject (such as pancreatic tissue samples, such as pancreatic cancer samples, or an infectious disease sample) can be compared to a control. In some embodiments, the control is a cancer sample (such as a pancreatic cancer sample) obtained from a subject or group of subjects known to have had good survival outcomes (or poor survival outcomes). In some embodiments, the control is an infectious disease sample obtained from a subject or group of subjects known to have the infectious disease. In other embodiments, the control is a standard or reference value based on an average of historical values. In some examples, the reference values are an average expression (such as RNA expression) value for each of a microbe- and/or cancer-related molecule (such as molecules useful for detecting microbes of one or more genera, such as genera Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia) and/or housekeeping genes, in a cancer sample (such as a pancreatic cancer sample) obtained from a subject or group of subjects known to have or to have had cancer. In other embodiments, the reference values are an average expression (such as RNA expression) value for each of an infectious disease-related molecule (such as molecules useful for detecting microbes of one or more genera, such as genera Candida, Helicobacter, Mycobacterium, or Salmonella, or molecules useful for detecting one or more viruses, such as a lentivirus, alphaherpesvirus, or coronavirus).

In some examples, the reference values are an average expression (such as RNA expression) value for each of NTHL1, LYPD2, MUC16, C2CD4B, FMO3, and IL1RL1 in a cancer sample (such as a pancreatic cancer sample) obtained from a subject or group of subjects known to have or to have had cancer, or a corresponding non-cancer sample of the same tissue type.

In some examples, the reference values are an average expression (such as RNA expression) value for each of the genes listed in Table 2 in T cells obtained from a subject or group of subjects known to have or to have had cancer (such as T cells from or near the tumor), or T cells from a subject known not to have cancer.

In some embodiments, the control is a non-cancer sample (such as a non-cancer sample of the same tissue type as the cancer) obtained from a subject or group of subjects known to not have cancer. In other embodiments, the control is a non-infectious disease sample obtained from a subject or group of subjects known to not have the infectious disease.

Tissue samples can be obtained from a subject, for example, from infectious disease patients or from cancer patients (such as pancreatic cancer patients) who have undergone tumor resection as a form of treatment. In some embodiments, cancer samples (such as pancreatic cancer samples) are obtained by biopsy. Biopsy samples can be fresh, frozen or fixed, such as formalin-fixed and paraffin embedded. Samples can be removed from a patient surgically, by extraction (for example by hypodermic or other types of needles), by microdissection, by laser capture, or by other means.

In some examples, the sample is used to generate a suspension of individual cells, such that nucleic acid molecules can be sequenced for individual cells. In some examples, individual cells are bar coded.

In some examples, proteins and/or nucleic acid molecules (e.g., DNA, RNA, miRNA, mRNA) are isolated or purified from the cancer sample (such as a pancreatic cancer sample) and non-cancer sample. In some examples, the cancer sample (such as a pancreatic cancer sample) is used directly, or is concentrated, filtered, or diluted. In other examples, proteins and/or nucleic acid molecules (e.g., DNA, RNA, miRNA, mRNA) are isolated or purified from the sample from the subject suspected of having the infectious disease and a control sample. In some examples, the sample from the subject suspected of having the infectious disease is used directly, or is concentrated, filtered, or diluted.

### Exemplary Methods of Detecting Expression

The disclosed methods include detecting expression of genes useful for identifying bacteria or fungi in a sample, such as in individual cells obtained from a tumor (or corresponding sample that is non-cancerous). The disclosed methods also include detecting expression of genes useful for identifying bacteria, fungi, or viruses, such as in a sample or individual cells obtained from a subject suspected of having an infectious disease. That is, sequencing is determined at the single-cell level. In certain embodiments detecting expression of such genes includes sequencing microbial nucleic acid molecules (such as by RNA-seq) in individual cells (such as by scRNA-seq) obtained from a subject.

Expression of nucleic acid molecules or proteins of microbes of one or more genera, such as genera Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter. Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; such as NTHL1, LYPD2, MUC16, C2CD4B, FMO3, and/or IL1RL1; and/or one or more genes of Table 2 can be detected alone or in combination in individual cells (e.g., cancer cells, non-cancer cells, T cells) using a variety of methods. Expression of nucleic acid molecules (e.g., total RNA, mRNA, tRNA, cDNA) or protein is contemplated herein.

Gene expression can be evaluated by detecting mRNA encoding the gene of interest. Thus, the disclosed methods can include evaluating mRNA encoding microbe- and/or cancer-related molecules (such as molecules useful for detecting microbes of one or more genera, such as genera Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2). The disclosed methods can also include evaluating mRNA encoding infectious disease-related molecules (such as molecules useful for detecting microbes of one or more genera, such as genera Candida, Helicobacter, Mycobacterium, or Salmonella, or molecules useful for detecting one or more viruses, such as a lentivirus, alphaherpesvirus, or coronavirus). In some examples, mRNA expression is quantified.

Exemplary methods for sequencing-based gene expression analysis include Serial Analysis of Gene Expression (SAGE), gene expression analysis by massively parallel signature sequencing (MPSS), and RNA sequencing (RNA-seq) analysis.

In one example, polymerase chain reaction (PCR) is used, such as RT-PCR can be used. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems, 850 Lincoln Centre Drive, Foster City, CA 94404 under the trademark ABI PRISM® 7700.

The primers used for the amplification are selected so as to amplify a unique segment of the gene of interest, such as RNA (such as mRNA) encoding microbe- and/or cancer-related molecules (such as molecules useful for detecting microbes of one or more genera, such as Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2; or molecules useful for detecting microbes, such as microbes of genera Candida, Helicobacter, Mycobacterium, or Salmonella; or molecules useful for detecting one or more viruses, such as an HIV virus, alphaherpesvirus, or coronavirus). In some embodiments, expression of other genes is also detected, such as other known cancer or infectious disease markers or housekeeping genes. Primers that can be used to amplify microbe- and/or cancer-related molecules (such as molecules useful for detecting microbes of one or more genera, such as Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2; or molecules useful for detecting microbes of genera, such as Candida, Helicobacter, Mycobacterium, or Salmonella; or molecules useful for detecting one or more viruses, such as an HIV virus, alphaherpesvirus, or coronavirus) are commercially available or can be designed and synthesized. In some examples, the primers specifically hybridize to a promoter or promoter region of a microbe- and/or cancer-related molecule (such as molecules useful for detecting microbes of one or more genera, such as Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2; or molecules useful for detecting microbes, such as microbes of genera Candida, Helicobacter, Mycobacterium, or Salmonella; or molecules useful for detecting one or more viruses, such as an HIV virus, alphaherpesvirus, or coronavirus). An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some embodiments of this method, the expression of a “housekeeping” gene or “internal control” can also be evaluated. These terms include any constitutively or globally expressed gene whose presence enables an assessment of mRNA levels provided herein. Such an assessment includes a determination of the overall constitutive level of gene transcription and a control for variations in RNA recovery. Exemplary housekeeping genes include tubulin, glyceraldehyde-3-phosphate-dehydrogenase (GAPDH), beta-actin, and 18S ribosomal RNA.

Serial analysis of gene expression (SAGE) allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997, herein incorporated by reference in their entireties).

In situ hybridization (ISH) is another method for detecting and comparing expression of microbe-and/or cancer-related molecules (such as molecules useful for detecting microbes of one or more genera, such as Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2; or molecules useful for detecting microbes, such as microbes of genera Candida, Helicobacter, Mycobacterium, or Salmonella; or molecules useful for detecting one or more viruses, such as an HIV virus, alphaherpesvirus, or coronavirus). ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of microbe- and/or cancer-related molecules (such as molecules useful for detecting microbes of one or more genera, such as Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2; or molecules useful for detecting microbes of genera such as Candida, Helicobacter, Mycobacterium, or Salmonella; or molecules useful for detecting one or more viruses, such as an HIV virus, alphaherpesvirus, or coronavirus). Sample cells or tissues can be treated to increase their permeability to allow a probe to enter the cells, such as a gene-specific probe for microbe- and/or cancer-related molecules (such as molecules useful for detecting microbes of one or more genera, such as Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2; or molecules useful for detecting microbes of genera such as Candida, Helicobacter, Mycobacterium, or Salmonella; or molecules useful for detecting one or more viruses, such as an HIV virus, alphaherpesvirus, or coronavirus). The probe is added to the treated cells, allowed to hybridize at pertinent temperature, and excess probe is washed away. The probe can be labeled, for example with a radioactive, fluorescent or antigenic tag, so that the probe's location and quantity in the tissue can be determined, for example using autoradiography, fluorescence microscopy or immunoassay. Probes can be designed such that the probes specifically bind a gene of interest because microbe- and cancer-related molecules (such as molecules useful for detecting microbes of one or more genera, such as Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia; NTHL1, LYPD2, MUC16, C2CD4B, FMO3, and/or IL1RL1; and/or one or more genes of Table 2; or molecules useful for detecting microbes such as microbes of genera Candida, Helicobacter, Mycobacterium, or Salmonella; or molecules useful for detecting one or more viruses, such as an HIV virus, alphaherpesvirus, or coronavirus) are known.

In situ PCR is the PCR-based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples can be fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products can be achieved by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Gene expression can also be detected and quantitated using the nCounter® technology developed by NanoString (Seattle, WA; see, for example, U.S. Pat. Nos. 7,473,767; 7,919,237; and 9,371,563, which are herein incorporated by reference in their entireties). The nCounter® analysis system utilizes a digital color-coded barcode technology that is based on direct multiplexed measurement of gene expression. The technology uses molecular “barcodes” and single molecule imaging to detect and count hundreds of unique transcripts in a single reaction. Each color-coded barcode is attached to a single target-specific probe corresponding to a gene of interest (such as a TACE-response gene). Mixed together with controls, they form a multiplexed CodeSet.

Each color-coded barcode represents a single target molecule. Barcodes hybridize directly to target molecules and can be individually counted without the need for amplification. The method includes three steps: (1) hybridization; (2) purification and immobilization; and (3) counting. The technology employs two approximately 50 base probes per mRNA that hybridize in solution. The reporter probe carries the signal;

the capture probe allows the complex to be immobilized for data collection. After hybridization, the excess probes are removed and the probe/target complexes are aligned and immobilized in the nCounter® cartridge. Sample cartridges are placed in the digital analyzer for data collection. Color codes on the surface of the cartridge are counted and tabulated for each target molecule. This method is described in, for example, U.S. Pat. No. 7,919,237; and U.S. Patent Application Publication Nos. 20100015607; 20100112710; 20130017971, which are herein incorporated by reference in their entireties. Information on this technology can also be found on the company's website (nanostring.com).

Gene expression can also be detected and quantitated using RNA sequencing (RNA-seq), such as single cell RNA-seq (scRNA-seq) (see Stark, et al., Nat Rev Genet. 2019;20, 631-656; Haque, et al., Genome Med. 2017;9(75)). RNA-seq is most frequently used for analyzing differential gene expression between samples. In traditional RNA-seq analyses, the process of analyzing differential gene expression via RNA-seq begins with RNA extraction (such as from a tumor sample, such as a pancreatic cancer sample), followed by mRNA enrichment or ribosomal RNA depletion. cDNA is then synthesized, and an adaptor-ligated sequencing library is prepared. The library is sequenced to a read depth of, for example, 10-30 million reads per sample on a high-throughput platform (such as an Illumina platform). The sequencing reads (most often in the form of FASTQ files) are computationally aligned and/or assembled to a transcriptome. The reads are most often mapped to a known transcriptome or annotated genome, matching each read to one or more genomic coordinates. This process is often accomplished using alignment tools such as STAR, TopHat, or HISAT, which each rely on a reference genome. If no genome annotation containing known exon boundaries is available (such as if a reference genome annotation is missing or is incomplete), or if reads are to be associated with transcripts rather than genes, aligned reads can be used in a transcriptome assembly step using tools such as StringTie or SOAPdenovo-Trans. Tools such as Sailfish, Kallisto, and Salmon can associate sequencing reads directly with transcripts, without the need for a separate quantification step. Next, reads that have been mapped to transcriptomic or genomic locations are quantified using tools such as RSEM, CuffLinks, MMSeq, or HTSeq, or the alignment-free direct quantification tools Sailfish, Kallisto, or Salmon. Quantification results are often combined into an expression matrix, with one row for each expression feature (gene or transcript) and one column for each sample, with values being read counts or estimated abundances. Samples are then filtered and normalized to account for differences in expression patterns, read depth, and/or technical biases. Significant changes in expression of individual genes and/or transcripts between sample groups are then statistically modeled using one or more of various tools and computational methods.

scRNA-seq enables the systematic identification of cell populations in a tissue. Short sequences or barcodes may be added during library preparation or by direct RNA ligation, before amplification, to mark a sequence read as coming from a specific starting molecule or cell, such as in scRNA-seq experiments. In a scRNA-seq analysis, a tissue sample (such as a pancreatic tissue sample, such as a pancreatic cancer tissue sample) is dissociated, single cells are separated, and RNA from each individual cell is converted to cDNA (and can be labelled during reverse transcription) and then amplified (typically using PCR) for sequencing. The synthesized cDNA is used as the input for library preparation. Amplified nucleic acids can also be labelled with barcodes (such as using single-cell combinatorial indexing RNA sequencing or split-pool ligation-based transcriptome sequencing). Tissue dissociation may be accomplished using methods known in the art, such as mechanical disaggregation and/or enzymatic dissociation, such as enzymatic dissociation using collagenase and/or DNase. Similarly, single cells can be separated using known methods, such as flow-cytometry, wherein cells can be flow-sorted directly into micro-plates containing lysis buffer. Individual cells can also be captured in microfluidic chips or loaded into nano-well devices (e.g., by Poisson distribution), isolated, and merged into droplets (containing reagents) via droplet- microfluidic isolation (such as Drop-Seq or InDrop). Isolated single cells are then lysed such that RNA can be released for cDNA synthesis.

### Methods of Treating Cancer in a Subject

Also disclosed are methods of treating a cancer in a subject. In some embodiments, the cancer is pancreatic cancer. In some embodiments, the cancer is lung cancer. Certain embodiments of the method include sequencing microbial nucleic acid molecules (such as by scRNA-seq) in individual cells obtained from the subject, classifying the subject as having the cancer when the presence of certain microbes is detected in the individual cells or in the sample, and, if the subject is determined to have the cancer, administering at least one of surgery, radiation therapy, targeted therapy, immunotherapy, a chemotherapeutic agent, antimicrobial, selective bacteriophage, or palliative care to the subject.

A subject who has been diagnosed with a cancer as described herein can be administered an agent or therapy by any chosen route. Administration can be acute and chronic administration and/or local and systemic administration. In some embodiments of the disclosed methods, administration of a therapeutic agent (such as chemotherapy, an antimicrobial, biologic, or a selective bacteriophage) is by injection (such as intravenous, intramuscular, subcutaneous, intradermal, intrathecal (such as lumbar puncture), intraosseous, intratumoral, or intraperitoneal). In some examples, administration of a therapeutic agent (such as chemotherapy, an antimicrobial, biologic, or a selective bacteriophage) is oral (such as sublingual), rectal, transdermal (such as topical), intranasal, vaginal, or by inhalation. In certain embodiments, chemotherapeutic agents include gemcitabine, 5-fluorouracil, oxaliplatin, capecitabine, cisplatin, irinotecan, liposomal irinotecan, paclitaxel, albumin-bound paclitaxel, or docetaxel, carboplatin, vinorelbine, folinic acid, or oxaliplatin, in any combination together or with other agents and/or therapies.

In one example, one or more antimicrobial agents are administered to the subject diagnosed with cancer using the disclosed methods, such as or more of amikacin, ampicillin, ampicillin-sulbactam, aztreonam, ceftazidime, ceftaroline, cefazolin, cefepime, ceftriaxone, ciprofloxacin, colistin, daptomycin, oxycycline, erythromycin, ertapenem, gentamicin, imipenem, linezolid, meropenem, minocycline, piperacillin-tazobactam, trimethoprim-sulfamethoxazole, tobramycin, and vancomycin. Additional antimicrobial agents that may be used include aminoglycosides (including but not limited to kanamycin, neomycin, netilmicin, paromomycin, streptomycin, and spectinomycin), ansamycins (including but not limited to rifaximin), carbapenems (including but not limited to doripenem), cephalosporins (including but not limited to cefadroxil, cefalotin, cephalexin, cefaclor, cefprozil, fecluroxime, cefixime, cefdinir, cefditoren, cefotaxime, cefpodoxime, ceftibuten, and ceftobiprole), glycopeptides (including but not limited to teicoplanin, telavancin, dalbavancin, and oritavancin), lincosamides (including but not limited to clindamycin and lincomycin), macrolides (including but not limited to azithromycin, clarithromycin, dirithromycin, roxithromycin, telithromycin, and spiramycin), nitrofurans (including but not limited to furazolidone and nitrofurantoin), oxazolidinones (including but not limited to posizolid, radezolid, and torezolid), penicillins (including but not limited to amoxicillin, flucloxacillin, penicillin, amoxicillin/clavulanate, and ticarcillin/clavulanate), polypeptides (including but not limited to bacitracin and polymyxin B), quinolones (including but not limited to enoxacin, gatifloxacin, gemifloxacin, levofloxacin, lomefloxacin, moxifloxacin, naldixic acid, norfloxacin, trovafloxacin, grepafloxacin, sparfloxacin, and temafloxacin), suflonamides (including but not limited to mafenide, sulfacetamide, sulfadiazine, sulfadimethoxine, sulfamethizole, sulfamethoxazole, sulfasalazine, and sulfisoxazole), tetracyclines (including but not limited to demeclocycline, doxycycline, oxytetracycline, and tetracycline), and others (including but not limited to clofazimine, ethambutol, isoniazid, rifampicin, arsphenamine, chloramphenicol, fosfomycin, metronidazole, tigecycline, and trimethoprim). Further antimicrobial agents include amphotericin B, ketoconazole, fluconazole, itraconazole, posaconazole, voriconazole, anidulafungin, caspofungin, micafungin, and flucytosine.

In one example, one or more antibiotics are administered to the subject diagnosed with cancer using the disclosed methods, such as or more of tetracycline-derived antibiotics such as, e.g., tetracycline, doxycycline, chlortetracycline, clomocycline, demeclocycline, lymecycline, meclocycline, metacycline, minocycline, oxytetracycline, penimepicycline, rolitetracycline, or tigecycline; amphenicol-derived antibiotics such as, e.g., chloramphenicol, azidamfenicol, thiamphenicol, or florfenicol; macrolide-derived antibiotics such as, e.g., erythromycin, azithromycin, spiramycin, midecamycin, oleandomycin, roxithromycin, josamycin, troleandomycin, clarithromycin, miocamycin, rokitamycin, dirithromycin, flurithromycin, telithromycin, cethromycin, tulathromycin, carbomycin A, kitasamycin, midecamicine, midecamicine acetate, tylosin (tylocine), or ketolide-derived antibiotics such as, e.g., telithromycin, or cethromycin; lincosamide-derived antibiotics such as, e.g., clindamycin, or lincomycin; streptogramin-derived antibiotics such as, e.g., pristinamycin, or quinupristin/dalfopristin; oxazolidinone-derived antibiotics such as, e.g., linezolid, or cycloserine; aminoglycoside-derived antibiotics such as, e.g., streptomycin, neomycin, framycetin, paromomycin, ribostamycin, kanamycin, amikacin, arbekacin, bekanamycin, dibekacin, tobramycin, spectinomycin, hygromycin B, paromomycin, gentamicin, netilmicin, sisomicin, isepamicin, verdamicin, astromicin, rhodostreptomycin, or apramycin; steroid-derived antibiotics such as, e.g., fusidic acid, or sodium fusidate; glycopeptide-derived antibiotics such as, e.g., vancomycin, oritavancin, telavancin, teicoplanin, dalbavancin, ramoplanin, bleomycin, or decaplanin; beta-lactam-derived antibiotics such as, e.g., amoxicillin, ampicillin, pivampicillin, hetacillin, bacampicillin, metampicillin, talampicillin, epicillin, carbenicillin, carindacillin, ticarcillin, temocillin, azlocillin, piperacillin, mezlocillin, mecillinam, pivmecillinam, sulbenicillin, benzylpenicillin, azidocillin, penamecillin, clometocillin, benzathine benzylpenicillin, procaine benzylpenicillin, phenoxymethylpenicillin, propicillin, benzathine, phenoxymethylpenicillin, pheneticillin, oxacillin, cloxacillin, dicloxacillin, flucloxacillin, meticillin, nafcillin, faropenem, biapenem, doripenem, ertapenem, imipenem, meropenem, panipenem, cefacetrile, cefadroxil, cefalexin, cefaloglycin, cefalonium, cefaloridine, cefalotin, cefapirin, cefatrizine, cefazedone, cefazaflur, cefazolin, cefradine, cefroxadine, ceftezole, cefaclor, cefamandole, cefminox, cefonicid, ceforanide, cefotiam, cefprozil, cefbuperazone, cefuroxime, cefuzonam, cefoxitin, cefotetan, cefmetazole, loracarbef, cefcapene, cefdaloxime, cefdinir, cefditoren, cefetamet, cefixime, cefmenoxime, cefodizime, cefoperazone, cefotaxime, cefpimizole, cefpiramide, cefpodoxime, cefsulodin, ceftazidime, cefteram, ceftibuten, ceftiolene, ceftizoxime, ceftriaxone, flomoxef, latamoxef, cefepime, cefozopran, cefpirome, cefquinome, ceftobiprole, aztreonam, tigemonam, sulbactam, tazobactam, clavulanic acid, ampicillin/sulbactam, sultamicillin, piperacillin/tazobactam, co-amoxiclav, amoxicillin/clavulanic acid, or imipenem/cilastatin; sulfonamide-derived antibiotics such as, e.g., acetazolamide, benzolamide, bumetanide, celecoxib, chlorthalidone, clopamide, dichlorphenamide, dorzolamide, ethoxzolamide, furosemide, hydrochlorothiazide, indapamide, mafenide, mefruside, metolazone, probenecid, sulfacetamide, sulfadiazine, sulfadimethoxine, sulfadoxine, sulfanilamides, sulfamethoxazole, sulfamethoxypyridazine, sulfasalazine, sultiame, sumatriptan, xipamide, zonisamide, sulfaisodimidine, sulfamethizole, sulfadimidine, sulfapyridine, sulfafurazole, sulfathiazole, sulfathiourea, sulfamoxole, sulfadimethoxine, sulfalene, sulfametomidine, sulfametoxydiazine, sulfaperin, sulfamerazine, sulfaphenazole, or sulfamazone; quinolone-derived antibiotics such as, e.g., cinoxacin, flumequine, nalidixic acid, oxolinic acid, pipemidic acid, piromidic acid, rosoxacin, ciprofloxacin, enoxacin, fleroxacin, lomefloxacin, nadifloxacin, ofloxacin, norfloxacin, pefloxacin, rufloxacin, balofloxacin, grepafloxacin, levofloxacin, pazufloxacin, sparfloxacin, temafloxacin, tosufloxacin, besifloxacin, clinafloxacin, garenoxacin, gemifloxacin, moxifloxacin, gatifloxacin, sitafloxacin, trovafloxacin, alatrofloxacin, prulifloxacin, danofloxacin, difloxacin, enrofloxacin, ibafloxacin, marbofloxacin, orbifloxacin, pradofloxacin, sarafloxacin, ecinofloxacin, or delafloxacin; imidazole-derived antibiotics such as, e.g., metronidazole; nitrofuran-derived antibiotics such as, e.g., nitrofurantoin, or nifurtoinol; aminocoumarin-derived antibiotics such as, e.g., novobiocin, clorobiocin, or coumermycin A1; ansamycin-derived antibiotics, including rifamycin-derived antibiotics such as, e.g., rifampicin (rifampin), rifabutin, rifapentine, or rifaximin; and also further antibiotics such as, e.g., fosfomycin, bacitracin, colistin, polymyxin B, daptomycin, xibornol, clofoctol, methenamine, mandelic acid, nitroxoline, mupirocin, trimethoprim, brodimoprim, iclaprim, tetroxoprim, or sulfametrole; without being limited thereto.

In one example, one or more antifungal agents are administered to the subject diagnosed with cancer using the disclosed methods, such as or more polyenes (for example, amphotericin B, candicidin, dennostatin, filipin, fungichromin, hachimycin, hamycin, lucensomycin, mepartricin, natamycin, nystatin, pecilocin, and perimycin), others (for example, azaserine, griseofulvin, oligomycins, neomycin undecylenate, pyrrolnitrin, siccanin, tubercidin, and viridin), allylamines (for example, butenafine, naftifine, and terbinafine), imidazoles (for example, bifonazole, butoconazole, chlordantoin, chlormiidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, and tioconazole), thiocarbamates (for example, tolciclate, tolindate, and tolnaftate), triazoles (for example, fluconazole, itraconazole, saperconazole, and terconazole), and others (for example, acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate).

In one example, one or more chemotherapeutic agents are administered to the subject diagnosed with cancer (such as pancreatic cancer) using the disclosed methods, such as or more of (such as 1, 2, 3 or 4 of) gemcitabine, 5-fluorouracil (5-FU), oxaliplatin, Albumin-bound paclitaxel, capecitabine, cisplatin, leucovorin, docetaxel, and irinotecan. In one example, a chemotherapy treatment for a pancreatic cancer analyzed using the disclosed methods includes gemcitabine, 5-FU, or capecitabine, such as fluorouracil, leucovorin, irinotecan, and oxaliplatin, (FOLFIRINOX). In one example, a chemotherapy treatment for a pancreatic cancer analyzed using the disclosed methods includes gemcitabine plus nab-paclitaxel. In one example, a chemotherapy treatment for a pancreatic cancer analyzed using the disclosed methods includes gemcitabine.

In one example, one or more chemotherapeutic agents are administered to the subject diagnosed with cancer (such as lung cancer, such as NSCLC) using the disclosed methods, such as or more of (such as 1, 2, 3 or 4 of) Cisplatin, Carboplatin, Paclitaxel, Albumin-bound paclitaxel (nab-paclitaxel), Docetaxel, Gemcitabine, vinorelbine, Etoposide, and Pemetrexed.

In one example, one or more biologic agents (e.g., mAbs) are administered (e.g., iv) to the subject diagnosed with cancer (such as pancreatic or lung cancer) using the disclosed methods, such as or more of (such as 1, 2, 3 or 4 of) a PD-1 inhibitor (e.g., nivolumab, pembrolizumab, and cemiplimab), PD-L1 inhibitor (e.g., atezolizumab and durvalumab), and CTLA4 inhibitor (e.g., ipilimumab).

### Methods of Treating Infectious Disease in a Subject

Also disclosed are methods of treating an infectious disease in a subject. Certain embodiments of the method include sequencing microbial nucleic acid molecules (such as by scRNA-seq) in individual cells obtained from the subject, identifying the infectious disease in the subject when the presence of certain microbes is detected in the individual cells or in the sample, and, if the subject is determined to have the infectious disease, administering at least one treatment to the subject.

A subject who has been diagnosed with an infectious disease as described herein can be administered an agent or therapy (such as an antibiotic, antifungal, or antiviral agent) by any chosen route. Administration can be acute or chronic administration and/or local and systemic administration. In some embodiments of the disclosed methods, administration of a therapeutic agent is intravenous, oral (such as sublingual), rectal, transdermal (such as topical), intranasal, vaginal, or by inhalation. Other supportive methods, such as intravenous fluids and oxygen, can also be administered.

In some examples, the subject is administered an antibiotic. Exemplary antibiotics that can be administered include In one example, one or more antimicrobial agents are administered to the subject diagnosed with cancer using the disclosed methods, such as or more of amikacin, ampicillin, ampicillin-sulbactam, aztreonam, ceftazidime, ceftaroline, cefazolin, cefepime, ceftriaxone, ciprofloxacin, colistin, daptomycin, oxycycline, erythromycin, ertapenem, gentamicin, imipenem, linezolid, meropenem, minocycline, piperacillin-tazobactam, trimethoprim-sulfamethoxazole, tobramycin, and vancomycin. Additional antimicrobial agents that may be used include aminoglycosides (including but not limited to kanamycin, neomycin, netilmicin, paromomycin, streptomycin, and spectinomycin), ansamycins (including but not limited to rifaximin), carbapenems (including but not limited to doripenem), cephalosporins (including but not limited to cefadroxil, cefalotin, cephalexin, cefaclor, cefprozil, fecluroxime, cefixime, cefdinir, cefditoren, cefotaxime, cefpodoxime, ceftibuten, and ceftobiprole), glycopeptides (including but not limited to teicoplanin, telavancin, dalbavancin, and oritavancin), lincosamides (including but not limited to clindamycin and lincomycin), macrolides (including but not limited to azithromycin, clarithromycin, dirithromycin, roxithromycin, telithromycin, and spiramycin), nitrofurans (including but not limited to furazolidone and nitrofurantoin), oxazolidinones (including but not limited to posizolid, radezolid, and torezolid), penicillins (including but not limited to amoxicillin, flucloxacillin, penicillin, amoxicillin/clavulanate, and ticarcillin/clavulanate), polypeptides (including but not limited to bacitracin and polymyxin B), quinolones (including but not limited to enoxacin, gatifloxacin, gemifloxacin, levofloxacin, lomefloxacin, moxifloxacin, naldixic acid, norfloxacin, trovafloxacin, grepafloxacin, sparfloxacin, and temafloxacin), suflonamides (including but not limited to mafenide, sulfacetamide, sulfadiazine, sulfadimethoxine, sulfamethizole, sulfamethoxazole, sulfasalazine, and sulfisoxazole), tetracyclines (including but not limited to demeclocycline, doxycycline, oxytetracycline, and tetracycline), and others (including but not limited to clofazimine, ethambutol, isoniazid, rifampicin, arsphenamine, chloramphenicol, fosfomycin, metronidazole, tigecycline, and trimethoprim) and combinations of two or more thereof. Specific antibiotics can be selected if the organism(s) causing the infection are identified. In some examples, the subject is treated with one or more broad-spectrum antibiotics immediately upon diagnosis, for example, prior to identifying a causative agent. The subject can then be administered one or more additional or different antibiotics when a specific causative agent is identified.

In other examples, the subject can be administered antiviral therapy, such as one or more of acyclovir, pocapavir, ganciclovir, emdesivir, galidesivir, arbidol, favipiravir, baricitinib, interferon, ribavirin, or lopinavir/ritonavir. In specific examples, the infectious disease is HIV, and the subject is administered antiretroviral agents, such as nucleoside and nucleotide reverse transcriptase inhibitors (nRTI), non-nucleoside reverse transcriptase inhibitors (NNRTI), protease inhibitors, entry inhibitors (or fusion inhibitors), maturation inhibitors, or broad spectrum inhibitors, such as natural antivirals. Exemplary agents include lopinavir, ritonavir, zidovudine, lamivudine, tenofovir, emtricitabine, and efavirenz.

In other examples, the subject can be administered antifungal therapy, such as one or more of polyenes (for example, amphotericin B, candicidin, dennostatin, filipin, fungichromin, hachimycin, hamycin, lucensomycin, mepartricin, natamycin, nystatin, pecilocin, and perimycin), others (for example, azaserine, griseofulvin, oligomycins, neomycin undecylenate, pyrrolnitrin, siccanin, tubercidin, and viridin), allylamines (for example, butenafine, naftifine, and terbinafine), imidazoles (for example, bifonazole, butoconazole, chlordantoin, chlormiidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, and tioconazole), thiocarbamates (for example, tolciclate, tolindate, and tolnaftate), triazoles (for example, fluconazole, itraconazole, saperconazole, and terconazole), and others (for example, acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate)

### EXAMPLES

The following examples are provided to illustrate certain particular features and/or embodiments. These examples should not be construed to limit the disclosure to the particular features or embodiments described.

Microorganisms are detected in multiple cancer types, including in tumors of the pancreas and other putatively sterile organs. However, it remains unclear whether bacteria and fungi preferentially associate with specific tissue contexts and whether they influence oncogenesis or anti-tumor responses in humans. SAHMI was developed herein as a novel framework to analyze host-microbiome interactions in the tumor microenvironment using single-cell sequencing data. Interrogating human pancreatic ductal adenocarcinomas (PDA) and nonmalignant pancreatic tissues identified an altered and diverse tumor microbiome, capturing both novel and known PDA-associated microbes detected with other technologies. Certain microbes showed preferential association with specific somatic cell-types, and their abundances correlated with select receptor gene expression and cancer hallmark activities in host cells. Nearly all tumor-infiltrating lymphocytes had infection-reactive transcriptional profiles, which may contribute to the lack of efficacy of immune checkpoint inhibitors. Pseudotime analysis suggested tumor-microbial co-evolution and identified three tumor modalities with distinct microbial, molecular, and clinical characteristics. Finally, using multiple independent datasets, a signature of increased intra-tumoral microbial diversity predicted patients at risk of poor survival. Collectively, tumor-microbiome cross-talk appears to modulate pancreatic cancer disease course with implications for clinical management.

### Example 1—Materials and Methods

SAHMI framework for detection of microbial entities from seRNAseq data: SAHMI (Single-cell Analysis of Host-Microbiome Interactions) was developed to estimate microbial diversity and to analyze patterns of human-microbiome interactions in tumor microenvironments at single cell resolution. SAHMI has four modules: (i) quantitation and annotation of microbial entities at multiple taxonomic levels from scRNAseq data with accompanying quality control filters; (ii) annotation of somatic cells and detection of preferential associations between microbial entities and host somatic cells; (iii) detection of significant associations between microbial profiles and the activities of signaling genes and cellular processes in host cells and at the tissue level; and (iv) analysis of associations between the sample microbiome and clinical attributes.

Annotation of somatic cells from scRNAseq data: SAHMI mapped the reads from single cell sequencing experiments to the host (e.g., human) genome and used the resulting transcriptomic signatures to cluster and annotate somatic cell types. Somatic cell clustering was done using the Seurat (Stuart et al. Cell, 177: 1888-1902.e21, 2019) R package with default parameters.

Quantitation and annotation of microbial entities: Metagenomic classification of paired-end reads from single-cell RNA sequencing fastq files was done using Kraken 2 (Wood et al. Genome Biol. 20: 257, 2019) with the default bacterial and fungal databases. The algorithm found exact matches of candidate 31-mer genomic substrings to the lowest common ancestor of genomes in a reference metagenomic database. Mapped metagenomic reads then underwent a series of filters. ShortRead (Morgan et al. Bioinformatics 25: 2607-2608, 2009) was used to remove low complexity reads (<20 non-sequentially repeated nucleotides), low quality reads (PHRED score<20), and PCR duplicates tagged with the same unique molecular identifier and cellular barcode. Non-sparse cellular barcodes were then selected by using an elbow-plot of barcode rank vs. total reads, smoothed with a moving average of 5, and with a cutoff at a change in slope<10⁻³, in a manner analogous to how cellular barcodes are typically selected in single-cell sequencing data (CellRanger (10× Genomics), Drop-seq Core Computational Protocol v2.0.0 (McCarroll laboratory)). Lastly, taxizedb (Chamberlain et al. Tools for Working with ‘Taxonomic’ Databases, 2020) was used to obtain full taxonomic classifications for all resulting reads, and the number of reads assigned to each clade was counted.

Normalization and identification of differentially expressed metagenomes: Sample-level normalized metagenomic levels were calculated as log2 (counts/total_counts*10,000+1). For analyses that compared cell-level metagenome and somatic gene expression, the default Seurat normalization was used. To identify bacterial and fungal genera that were differentially present in case samples compared to controls, a linear model was constructed to predict sample-level normalized genera levels as a function of tissue status, somatic cellular composition (to account for potential tropisms), and total metagenomic reads. Cellular counts and total metagenomic counts were log-normalized prior to model fitting.

Microbe-gene/pathway association: Correlations were done on three levels: (1) between microbe and gene or pathway levels within individual cells grouped by cell-type, (2) between the average microbe and gene or pathway level in a given cell-type, and (3) between total sample microbe levels and gene expression. Under the default SAHMI settings, at the individual cell-level, correlations were only done between microbes and somatic genes that were co-expressed in at least 50 of the same cell-type. Kyoto Encyclopedia of Genes and Genomes (KEGG) (Kanehisa et al. Nucleic Acids Res. 45: D353-D361, 2017) pathway enrichments from cell-level gene correlations were calculated for significant correlations with |r|>0.5 and adjusted p-value<0.05 using clusterProfiler (Yu et al. Omi. A J. Integr. Biol. 16: 284-287, 2012). Correlations between microbe levels and KEGG pathway scores were also examined at the individual cell and averaged-cell type levels. Pathway scores were calculated as the mean of root-mean scaled normalized gene expression to avoid a single-gene dominating a pathway score. Pathway scores in a cell-type were only calculated for pathways in which at least half the genes were detected.

Microbiome-host-cell composite pathways networks: Microbiome and pathway association data were used to construct an interaction network using igraph (Csardi et al. InterJournal Complex Syst. 1695: 1696, 2006) in which nodes were either averaged cell-type specific microbe levels or KEGG pathway scores, and edges represented significant correlations.

Pseudotime inferences: SAHMI uses a minimum spanning tree-based approach (Trapnell et al. Nat. Biotechnol. 32: 381-386, 2014) to order entire tissue microenvironments based on their cellular counts, KEGG pathway activities, and microbiome abundances. Cell counts were log lp normalized and scaled. Microbes were included if they were found to be differentially present in either tumors or control samples and if their abundance was >10⁻³ or if they were custom selected. Microbiome abundances per sample were normalized as stated above, centered, and unit-scaled. Normalized and scaled cell counts, pathway scores, and microbiome abundances for all samples were combined into a single matrix and used as input to Monocle's pseudotime functions (Trapnell et al. Nat. Biotechnol. 32: 381-386, 2014), using expressionFamily=uninormal( ) and norm_method=“none”. Numerical microbiome and clinical parameters were compared across the resulting states using a t-test, and categorical parameters using Fisher's test.

Survival and clinical covariate analyses: The microbiome Shannon diversity index was calculated for each sample, and the samples were divided according to whether the microbiome Shannon index was greater than the mean index for the cohort (classified as “high” diversity) or less than (classified as “low” diversity). Patients were stratified by their predicted microbial diversity, and the survminer package (github.com/kassambara/survminer/) was used to test the relationship with survival.

Cohort selection and metagenomic inferences: Single-cell RNA sequencing data were obtained for 24 human pancreatic ductal adenocarcinomas (PDA) and 11 control pancreas tissues (non-PDA lesions) from (Peng et al. Cell Res. 29(9):725-738, 2019). In that cohort, pancreatic tumor or tissue samples were collected during pancreatectomies or pancreatoduodenectomies (Table 1, patient characteristics). The samples were checked for batch effects at the levels of sample and somatic cell type clusters. The cohort had 100-500 million reads per sample, of which a substantial proportion did not map to the human genome, and these reads were used for metagenomic analyses. scRNAseq data from two additional studies that focused on the normal pancreas (Baron et al. Cell Syst. 3: 346-360.e4, 2016; Muraro et al. Cell Syst. 3: 385-394.e3, 2016) were obtained and processed similarly. Data were also obtained on microbial genera classified from bulk-RNA sequencing of pancreatic adenocarcinoma (PAAD) from TCGA (Poore et al. Nature 579: 567-574, 2020) (selecting counts and normalized expression values of TCGA genera passing all decontamination steps), and genera classified from 16S rRNA sequencing of pancreatic cancer in a recent large-scale study (Nejman et al. Science, 368(6494):973-980, 2020) (normalized expression of genera passing all filters except the multi-study filter). Decontamination was done by comparing genera identified in one sample to those identified in other scRNAseq data of the same organ type, or to those identified by Poore et al. (2020) in TCGA or by Nejman et al. (2020) from 16s-rRNA sequencing of the same organ type. Genera found exclusively in the sample being analyzed were identified as possible contaminants and were removed from further analyses.

TABLE 1
Clinical characteristics of PDA patients and control samples profiled by scRNA-seq. (Peng et al. Cell Res. 29(9): 725-738, 2019).

| | | | | | | | | Max | | | | | |
|---|---|---|---|---|---|---|---|---|---|---|---|---|---|
| | Pathologic | | Age | CA19-9 | | | | Diameter | TNM | | P | | P |
| Sample | Diagnosis | Sex | (years) | (U/ml) | DM | Procedure | Location | (mm) | Classification | Staging | Inv | VI | Inf |
| T1 | moderately-poorly | M | 64 | 86 | N | LDP | body | 26 | T4N2M0 | III | Y | Y | Y |
| | differentiated PDAC | | | | | | | | | | | | |
| T2 | well differentiated | M | 52 | 46.3 | N | PD | head | 20 | T1cN1M0 | IIB | Y | N | Y |
| | PDAC | | | | | | | | | | | | |
| T3 | moderately-poorly | F | 58 | 49.2 | Y | PD | uncinate | 22 | T2N0M0 | IB | N | N | Y |
| | differentiated PDAC | | | | | | process | | | | | | |
| T4 | moderately | F | 72 | 40.4 | Y | LDP | body | 14 | T1cN1M0 | IIB | N | N | Y |
| | differentiated PDAC | | | | | | | | | | | | |
| T5 | well-moderately | F | 65 | 37 | Y | PD | uncinate | 29 | T2N0M0 | IB | N | N | Y |
| | differentiated PDAC | | | | | | process | | | | | | |
| T6 | moderately-poorly | M | 64 | 155.1 | N | ODP | tail | 91 | T3N0M0 | IIA | N | N | Y |
| | differentiated PDAC | | | | | | | | | | | | |
| T7 | moderately | M | 70 | <0.6 | Y | ODP | body | 80 | T3N1M0 | IIB | N | N | Y |
| | differentiated PDAC | | | | | | | | | | | | |
| T8 | moderately-poorly | F | 66 | 82.5 | N | PD | uncinate | 17 | T1cN2M0 | III | N | N | N |
| | differentiated PDAC | | | | | | process | | | | | | |
| T9 | moderately-poorly | M | 36 | 11.2 | N | PD | head | 26 | T2N0M0 | IIA | Y | Y | Y |
| | differentiated PDAC | | | | | | | | | | | | |
| T10 | poorly differentiated | M | 61 | 972.8 | Y | PD | uncinate | 40 | T2N1M0 | IB | Y | Y | Y |
| | PDAC | | | | | | process | | | | | | |
| T11 | moderately-poorly | M | 51 | 211.1 | N | ODP | body and | 76 | T3N1M0 | IIB | Y | Y | Y |
| | differentiated PDAC | | | | | | tail | | | | | | |
| T12 | poorly differentiated | M | 54 | 146.1 | N | PD | uncinate | 50 | T3N2M0 | III | Y | Y | Y |
| | PDAC | | | | | | process | | | | | | |
| T13 | moderately-poorly | F | 58 | 21.9 | Y | PD | head | 30 | T2N1M0 | IIB | Y | N | Y |
| | differentiated PDAC | | | | | | | | | | | | |
| T14 | well differentiated | F | 67 | 77 | Y | PD | head | 33 | T2N1M0 | IIB | Y | Y | Y |
| | PDAC | | | | | | | | | | | | |
| T15 | well differentiated | F | 54 | 18.4 | N | LPD | head | 23 | T2N1M0 | IIB | Y | N | Y |
| | PDAC | | | | | | | | | | | | |
| T16 | poorly differentiated | F | 56 | 42.9 | N | LDP | body | 30 | T2N1M0 | IIB | Y | Y | Y |
| | PDAC | | | | | | | | | | | | |
| T17 | moderately | F | 71 | 209.3 | N | LDP | body and | 30 | T2N0M0 | IB | Y | N | N |
| | differentiated PDAC | | | | | | tail | | | | | | |
| T18 | moderately-poorly | F | 68 | 112.3 | Y | ODP | body | 28 | T2N0M0 | IB | Y | Y | Y |
| | differentiated PDAC | | | | | | | | | | | | |
| T19 | well-moderately | F | 59 | 93.9 | N | LPD | head | 35 | T2N0M0 | IB | Y | Y | Y |
| | differentiated PDAC | | | | | | | | | | | | |
| T20 | moderately | M | 59 | 2.2 | N | PD | head | 43 | T3N1M0 | IIB | Y | Y | Y |
| | differentiated PDAC | | | | | | | | | | | | |
| T21 | moderately-poorly | M | 59 | 528.6 | Y | LPD | head | 35 | T2N0M0 | IB | Y | Y | Y |
| | differentiated PDAC | | | | | | | | | | | | |
| T22 | moderately | F | 67 | 234.5 | N | ODP | body | 27 | T2N0M0 | IB | Y | N | Y |
| | differentiated PDAC | | | | | | | | | | | | |
| T23 | moderately-poorly | M | 54 | 312.2 | Y | PD | head | 27 | T2N1M0 | IIB | Y | Y | Y |
| | differentiated PDAC | | | | | | | | | | | | |
| T24 | moderately | F | 44 | 14.4 | N | PD | head | 20 | T1cN0M0 | IB | Y | N | Y |
| | differentiated PDAC | | | | | | | | | | | | |
| N1 | normal | F | 64 | 7.5 | N | ODP | tail | 50 | NA | NA | N | N | N |
| | pancreas/mucinous | | | | | | | | | | | | |
| | cystic neoplasia | | | | | | | | | | | | |
| N2 | normal | M | 55 | 171.2 | N | PPPD | descending | 11 | NA | NA | N | Y | N |
| | pancreas/small | | | | | | duodenum | | | | | | |
| | intestine papillary | | | | | | | | | | | | |
| | adenocarcinoma | | | | | | | | | | | | |
| N3 | normal | M | 50 | 6.4 | N | PD | descending | 20 | NA | NA | N | N | N |
| | pancreas/duodenal | | | | | | duodenum | | | | | | |
| | intraepithelial | | | | | | | | | | | | |
| | neoplasia | | | | | | | | | | | | |
| N4 | normal | M | 53 | 4.5 | N | LDP | body and | 40 | NA | NA | N | N | N |
| | pancreas/pancreatic | | | | | | tail | | | | | | |
| | neuroendocrine | | | | | | | | | | | | |
| | tumor | | | | | | | | | | | | |
| N5 | normal | F | 52 | 9 | N | LDP | body and | 24 | NA | NA | N | N | N |
| | pancreas/serous | | | | | | tail | | | | | | |
| | cystic neoplasia | | | | | | | | | | | | |
| N6 | normal | F | 31 | 29.5 | N | ODP | body | 22 | NA | NA | N | N | N |
| | pancreas/solid | | | | | | | | | | | | |
| | pseudopapillary | | | | | | | | | | | | |
| | tumor | | | | | | | | | | | | |
| N7 | normal | F | 42 | 12.7 | N | LDP | tail | 94 | NA | NA | N | N | N |
| | pancreas/mucinous | | | | | | | | | | | | |
| | cystic neoplasia | | | | | | | | | | | | |
| N8 | normal | M | 41 | 6 | N | LDP | body and | 76 | NA | NA | N | N | N |
| | pancreas/solid | | | | | | tail | | | | | | |
| | pseudopapillary | | | | | | | | | | | | |
| | tumor | | | | | | | | | | | | |
| N9 | normal | M | 34 | 23.8 | N | LDP | tail | 22 | NA | NA | N | N | N |
| | pancreas/pancreatic | | | | | | | | | | | | |
| | neuroendocrine | | | | | | | | | | | | |
| | tumor | | | | | | | | | | | | |
| N10 | normal | F | 65 | 193.3 | N | PD | common | NA | T3N0M0 | IIA | N | N | N |
| | pancreas/choledochal | | | | | | bile duct | | | | | | |
| | neuroendocrine | | | | | | | | | | | | |
| | tumors | | | | | | | | | | | | |
| N11 | normal | F | 30 | NA | N | LDP | body | 33 | NA | NA | N | N | N |
| | pancreas/solid | | | | | | | | | | | | |
| | pseudopapillary | | | | | | | | | | | | |
| | tumor | | | | | | | | | | | | |

DM: Diabetes Mellitus; LDP: Laparoscopic distal pancreatectomy; ODP: Open distal pancreatectomy; PD: Pancreatoduodenectomy; LPD: Laparoscopic pancreatoduodenectomy; PPPD: Pylorus preserved pancreatoduodenectomy; P Inv: Perineural Invasion; VI: Vascular Invasion; P Inf: Peripancreatic Infiltration.

Quality control analysis, comparative analyses, and benchmarking: To mitigate the influence of classification errors, contamination, noise, and batch effects, total genus abundances were examined, and genera sequenced with different technologies across multiple studies were compared. Specifically, metagenomes from the (Peng et al. Cell Res. 29(9):725-738, 2019) cohort were compared to those from (i) two other single-cell studies of the normal pancreas (Baron et al. Cell Syst. 3: 346-360.e4, 2016; Muraro et al. Cell Syst. 3: 385-394.e3, 2016) classified using our pipeline, (ii) genera classified from bulk-RNA sequencing of the TCGA pancreatic cancer (TCGA-PAAD) (Poore et al. Nature, 579: 567-574, 2020), and (iii) genera classified from 16S rRNA sequencing of pancreatic cancer (Nejman et al. Science, 368(6494):973-980, 2020), as described above. Genera in the single-cell datasets were only retained if they were present at a frequency greater than 10⁻⁴ and if they were detected in two or more independent studies. Pancreas-specific taxa were retained regardless of country of origin or other possible batch effects, although this approach risks filtering out individual specific or low-prevalence taxa.

To compare filtered microbial profiles across studies, the overlap coefficient of any two sets was calculated as overlap(X, Y)=intersect(X, Y)/min(|X|, |Y|). Study-level microbial abundances were compared with Spearman correlations and microbial detection was compared with the overlap coefficient. Harmonic mean p-values for combining dependent Spearman correlation associated p-values were calculated using the harmonicmeanp package (Wilson, Proc. Natl. Acad. Sci. 116(4): 1195-1200, 2019). Literature reported microbial changes in pancreatic disease were obtained from Table 1 in (Thomas et al. Nat. Rev. Gastroenterol. Hepatol. 17: 53-64, 2020). A list of putative laboratory contaminants was obtained from (Poore et al. Nature 579: 567-574, 2020), who performed extensive statistical analysis and literature research to identify common contaminants.

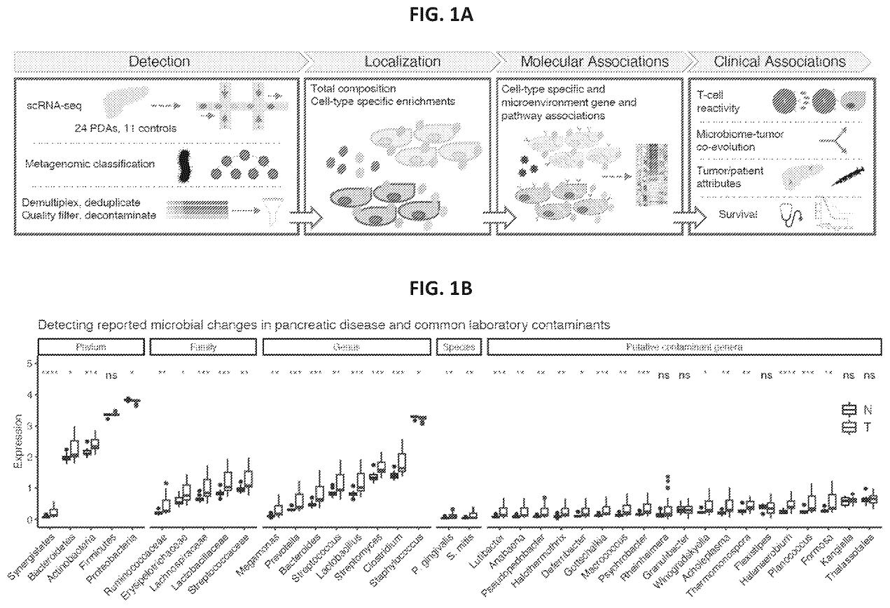

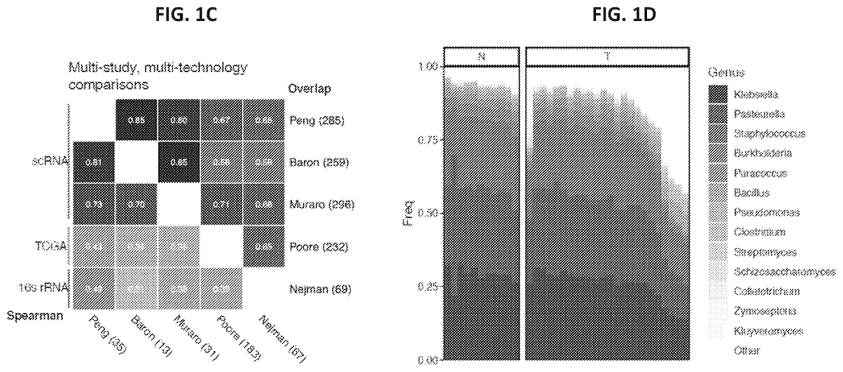

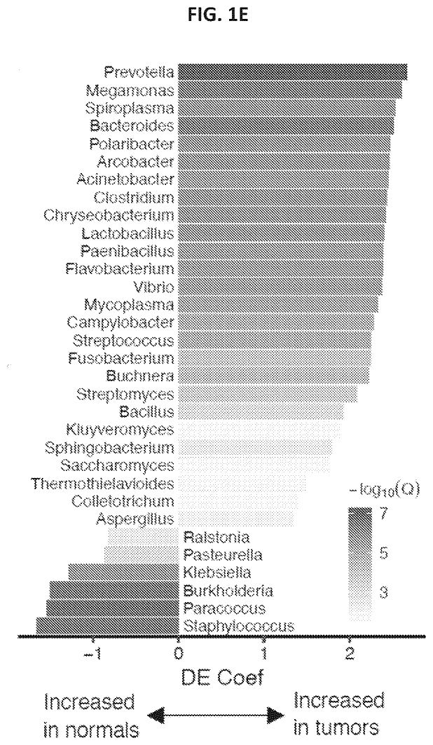

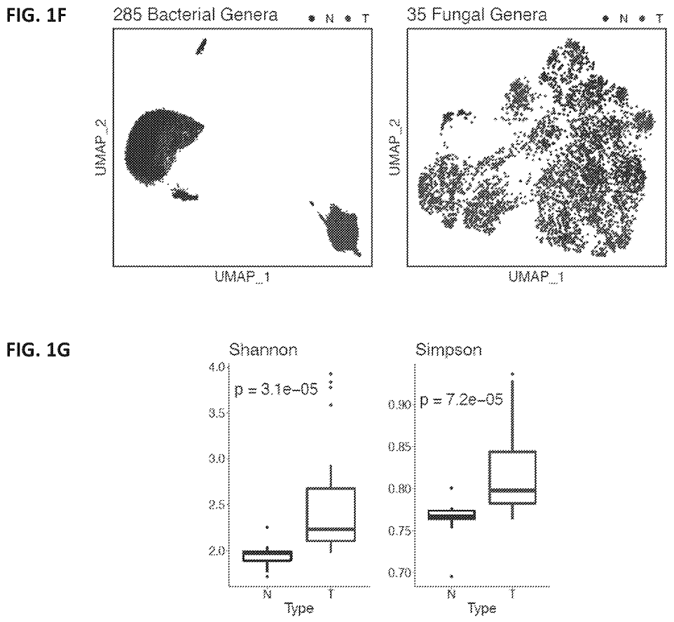

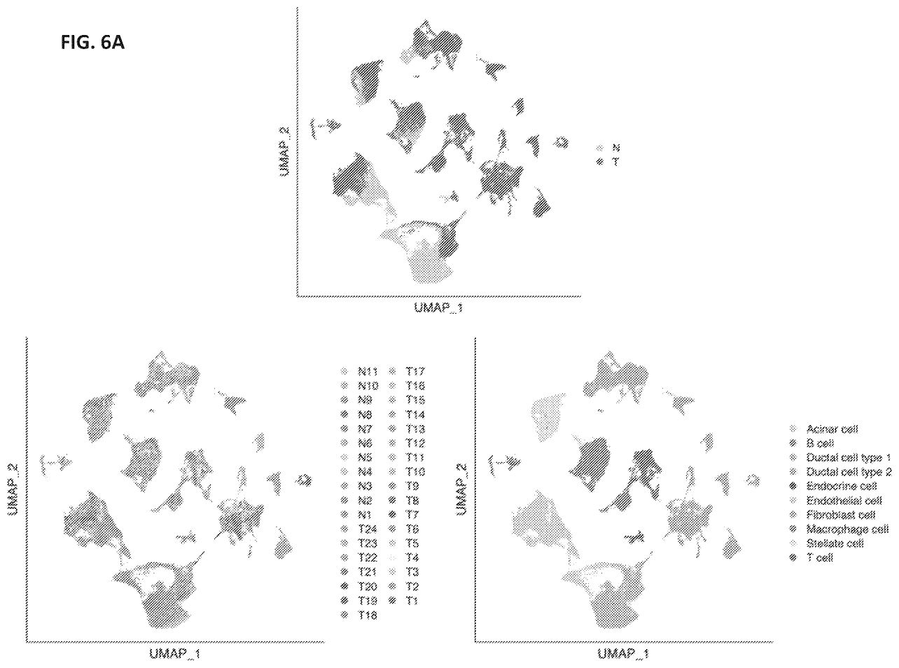

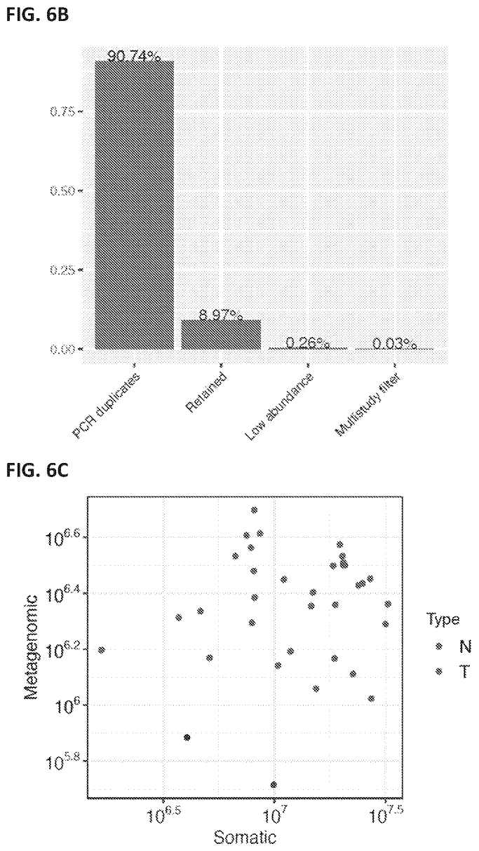

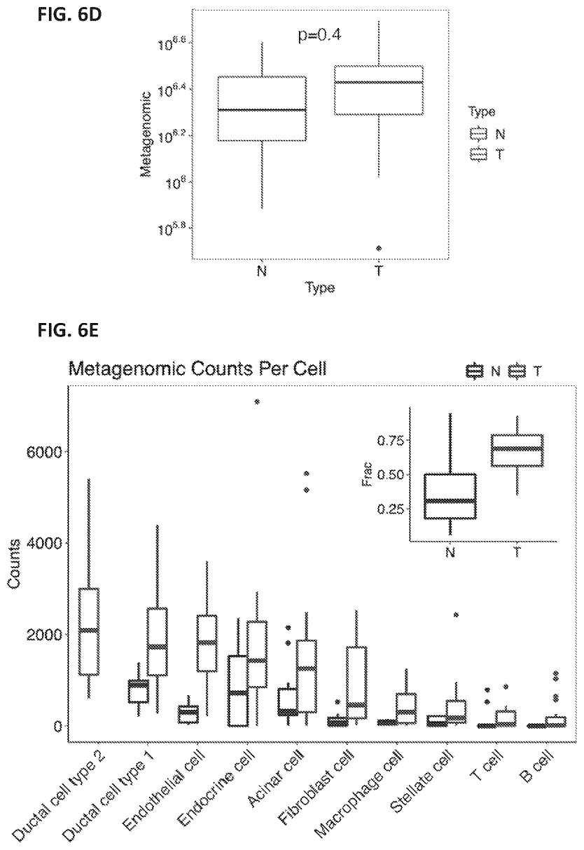

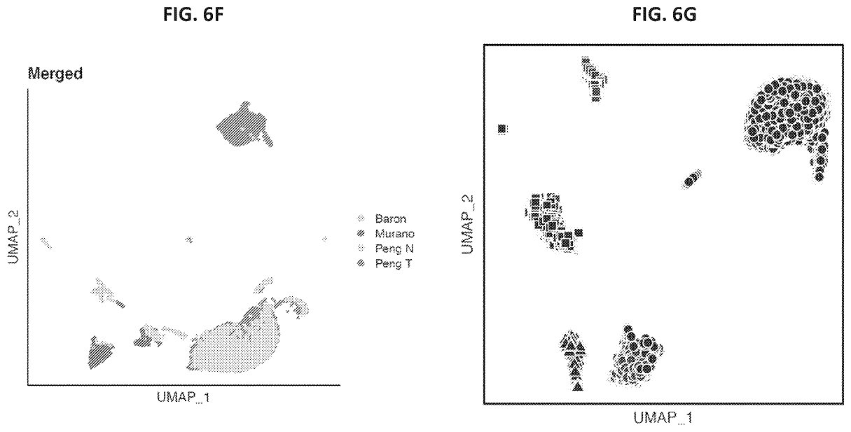

Metagenomic differences between tumor and non-tumor samples: As described above, SAHMI was used for normalization and identification of differentially expressed metagenomes between pancreatic tumors and non-malignant samples. Cellular counts and total metagenomic counts were log-normalized prior to model fitting. Tissue status was modeled as three groups: normal, tumor group 1 (tumors whose microbiome appeared broadly similar to that of nonmalignant samples), and tumor group 2 (tumors with markedly different microbiomes). These three groups were defined based on barcode clustering in the bacterial (FIG. 1F) and combined bacterial and fungal UMAP plots (FIG. 6G). Differentially present genera were identified as those with nonzero tissue-status coefficients (adjusted p<0.05). Figures in which differentially expressed genera are highlighted include statistically significant genera with either abundances >10⁻³ or literature-reported microbial associations to pancreatic cancer summarized in a recent review (Thomas et al. Nat. Rev. Gastroenterol. Hepatol. 17: 53-64, 2020).

Somatic cell-type and sample cellular composition predictions: Somatic cell clustering was done by SAHMI as described above. The somatic gene expression count matrix and cell type annotations were taken from the original study (Peng et al. Cell Res. 29(9):725-738, 2019). To ensure that gene count data were consistent regardless of the preprocessing pipeline, for five samples, gene counts were derived from raw fastq files using the Drop-seq Core Computational Protocol v2.0.0 from the McCarroll laboratory with default parameters. Briefly, barcodes with low quality bases were filtered out, the resulting transcripts were aligned to GRCH37 using the splice-aware STAR aligner (Dobin et al. Bioinformatics, 29: 15-21, 2013), and gene-level counts and cell-containing barcodes were estimated. Somatic cell clusters were then obtained using Seurat and were compared to those from the (Peng et al. Cell Res. 29(9):725-738, 2019) processed data and showed no major differences.

Identifying somatic cellular sub-clusters was done using the self-assembling manifolds (SAM) (Tarashansky et al. Elife, 8: 1-29, 2019) package in Python, which reduces the dimensionality of a dataset using an iterative approach that emphasizes features that discriminate across clusters. Each somatic cell-type was processed independently, whereby SAM reduced the data dimensionality and Seurat was used to find clusters in the resulting principal component reduction, using resolution=0.4 to capture only the major sub-clusters that were made of multiple samples. SAM was chosen because of its demonstrated good performance and because it produced interpretable sub-clusters, which were annotated using known markers.

Barcode cell-type predictions were done for the subset of cell-associated barcodes (13,848/23,546 total). Barcodes were identified as cell-associated if the same microbiome-tagging barcode also tagged somatic cellular RNA and was retained during analysis of the host-cells and assigned a cell-type label based on its somatic gene expression signatures. A random forest model was then trained to classify each barcode's associated somatic cell type based on its microbiome profile. To account for the large cell-type class imbalance in microbiome-tagging barcodes during model training (the majority of microbiome reads co-localized with epithelial and endothelial cells and few with immune cells), 150 barcodes from each cell-type were selected for training, and then the resulting model was used to predict the remaining 11,984 barcodes. Receiver-operator curves were calculated using the Proc (Robin et al. BMC Bioinformatics, 12: 77, 2011) R package. Multiple run of this procedure produced nearly identical receiver-operator curves.

Tumor microenvironment somatic cellular composition was predicted using least absolute shrinkage and selection operator (LASSO) linear regression from the glmnet (Simon et al. J. Stat. Software, 39(5): 1-13, 2011) R package. The model underwent 10-fold cross-validation using the ‘cv.glmnet’ function over a range of lambdas from exp(−0.5, −3) and alpha=1. LASSO regression with the same optimization parameters was also attempted 500 times to predict sample-label shuffled data.

Validation of cell-type enrichments across datasets: Metagenomic enrichments in somatic cell-types were determined using the FindAllMarkers function in Seurat, which calculates log-fold changes of normalized bacterial or fungal levels in each cell-type relative to all others and associated enrichment p-values using Wilcoxon rank-sum tests. To assess the significance and reproducibility of these enrichments, for two pancreatic single-cell datasets (Peng et al. Cell Res. 29(9):725-738, 2019; Baron et al. Cell Syst. 3: 346-360.e4, 2016), 80% of the cells were subsampled, the total number of statistically significant microbiome-cell-type enrichments were found, and then the cell-type labels and similarly calculated enrichments were randomized. This was repeated 500 times, and the distributions of the total number of enrichments found in each dataset from actual vs. shuffled data were compared, as well as the number of shared enrichments, using the Wilcoxon test.

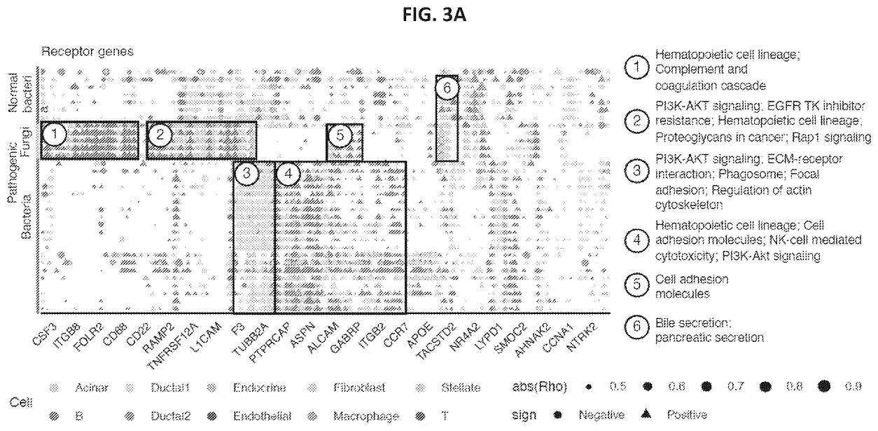

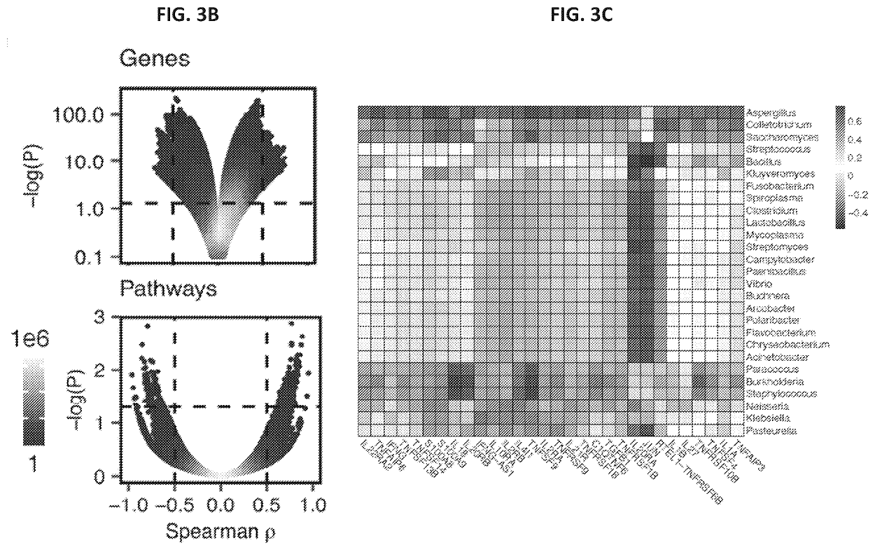

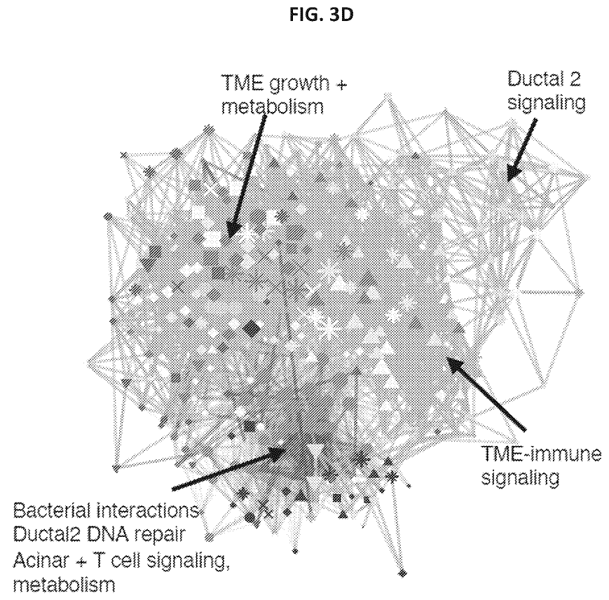

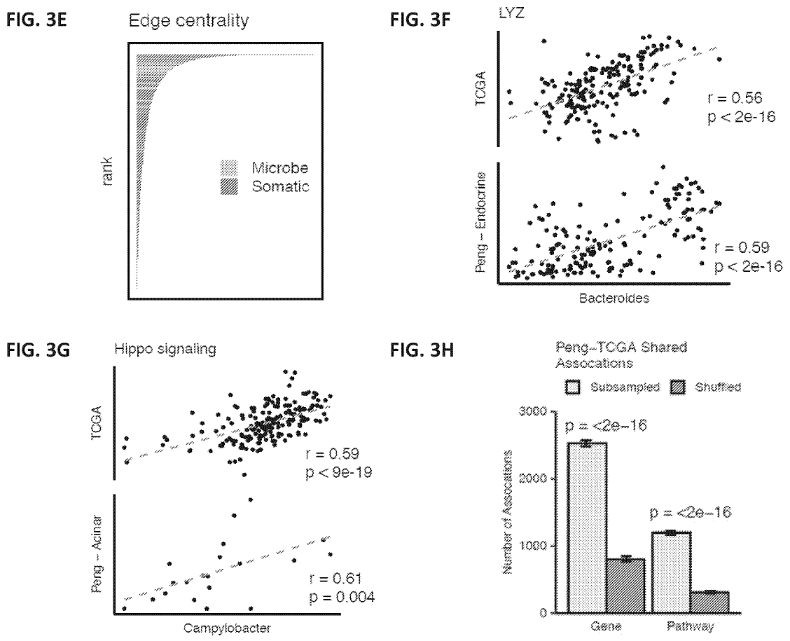

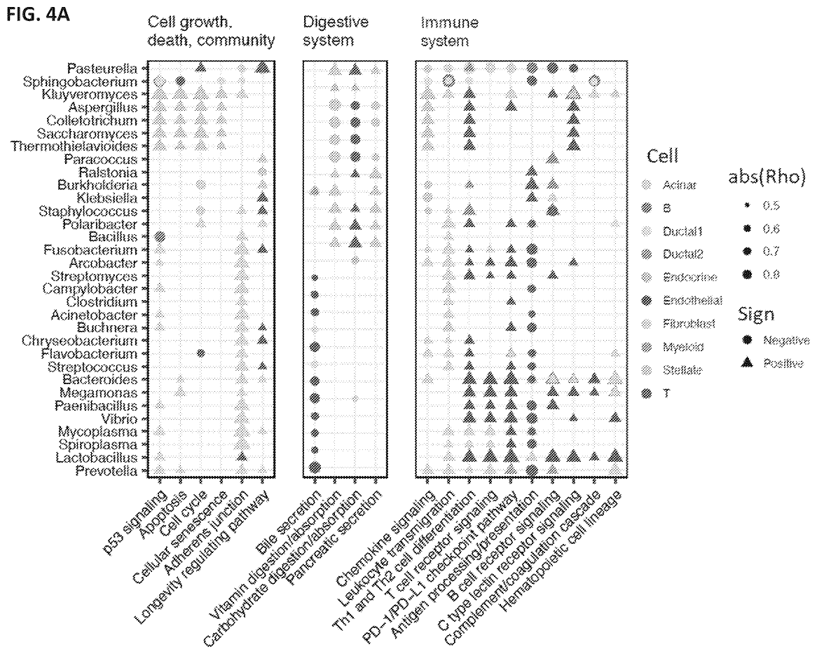

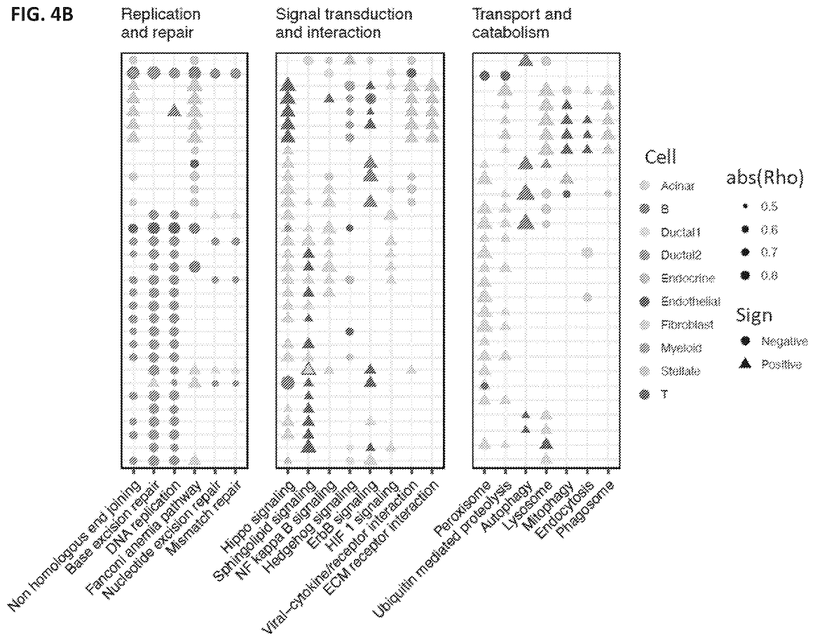

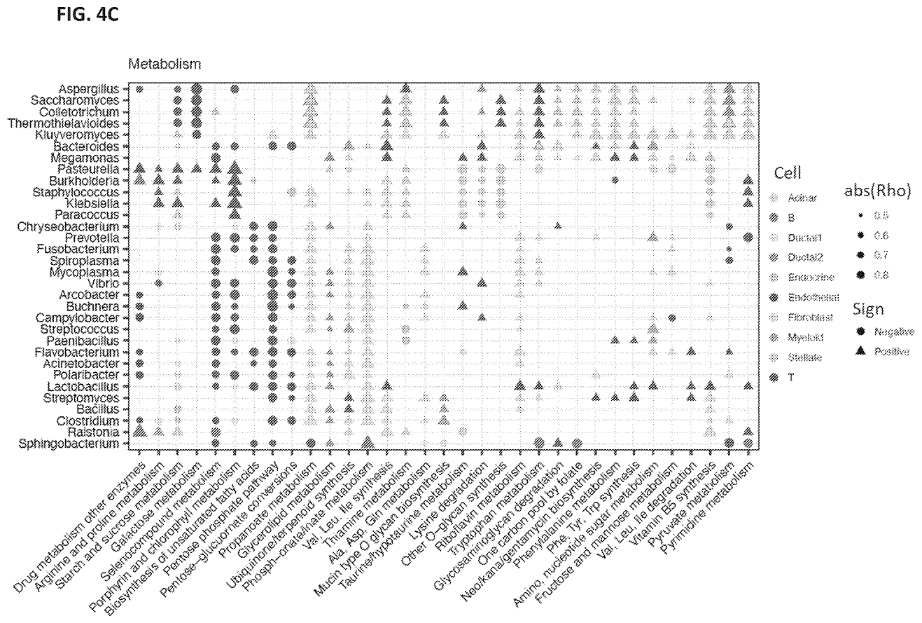

Association between microbes and cellular processes: Associations between microbial entities and cellular processes were analyzed in pancreatic tumors and non-malignant samples as stated above. Microenvironment-level correlations were examined between total microbes and inflammatory or antimicrobial genes. Inflammatory genes were obtained from (Smillie et al. Cell 178: 714-730.e22, 2019) and receptor and antimicrobial genes were obtained from GeneCards (Stelzer et al. Curr. Protoc. Bioinforma. 54: 1.30.1-1.30.33, 2016). Pathway score correlations in FIG. 4A-4C were grouped by KEGG groupings, and data were collected for pathways relevant to pancreatic function and cancer hallmarks; these pathways were: cell growth, death, community, digestive system, immune system, replication and repair, signal transduction and interaction, transport and catabolismand metabolism. Only pancreas or cancer-related pathways shown in FIG. 4A-4C were included in the FIG. 3D network. Microbe-cell-specific pathway edges were included if the correlation had a Spearman coefficient |r|>0.5 and adjusted p-value<0.05. Because some KEGG pathways can be inter-related or include overlapping gene sets, pathway-pathway edges were included between pathways correlated with Spearman |r|>0.75 and adjusted p-value<0.05. Edge centrality was calculated using igraph (Csardi et al. InterJournal Complex Syst. 1695: 1696, 2006).

Validation of microbe-gene and pathway associations: The significant correlations between microbes and genes and pathways found in the (Peng et al. Cell Res. 29(9): 725-738, 2019) cohort were compared to correlations between gene expression or pathways scores from the pancreatic cancer samples in the TCGA and the affiliated microbiome levels estimated by (Poore et al. Nature 579: 567-574, 2020). Normalized gene expression data for TCGA pancreatic cancer (PAAD) samples were obtained via RTCGAToolbox (Samur, PLOS One 9: e106397, 2014). A small number of common microbe-gene/pathway correlations were identified with Spearman |r|>0.5 and adjusted p-value<0.05 at both the individual cell level and the averaged cell-type level in (Peng et al. Cell Res. 29(9):725-738, 2019) compared to TCGA. The number of common statistically significant (t-test, p<0.05) microbe-gene/pathway correlations in Peng vs. TCGA were compared, regardless of correlation strength. In 500 iterations, 80% of both datasets were subsampled, averaged cell-type microbe and gene or pathway levels in (Peng et al. Cell Res. 29(9):725-738, 2019) and microbe and bulk gene or pathway levels in TCGA were correlated, and the number of statistically significant correlations shared by both datasets was calculated. This process was repeated with shuffled sample labels and the distributions of common correlations were compared using Wilcoxon testing in subsampled vs. shuffled data.

T-cell microenvironment reaction analysis: A random forest model was trained and validated to classify infection microenvironment reactive (IMER) vs. tumor microenvironment reactive (TMER) T-cells based on their gene expression profiles. The model was trained using single-cell RNA sequencing data of T-cells isolated from peripheral blood mononuclear cells from patients with bacterial sepsis (singlecell.broadinstitute.org/single_cell; SCP548) or from primary lung adenocarcinomas (E-MTAB-6149), which were previously shown to have low microbiome burden (Poore et al. Nature 579: 567-574, 2020; Nejman et al. Science, 368(6494):973-980, 2020). Processed gene expression data were analyzed using Seurat (Stuart et al. Cell. 177: 1888-1902.e21, 2019); cells were clustered based on transcriptomic profiles, and T-cells were identified using known markers (Nirmal et al. Cancer Immunol. Res. 6(11): 1388-1400, 2018). The FindAllMarkers function from Seurat was used to identify ˜500 genes differentially expressed in T-cells from lung cancer and sepsis patients. 1000 T-cells from each study were subsampled and the rank order of the ˜500 differentially expressed genes was used to train a random forest model to classify TMER or IMER T-cells. The model was then validated using the remaining T-cells from the lung cancer and sepsis studies, as well as 6 other datasets with either known microbial stimulation or cancer with low-microbiome burden: bladder cancer (GSE149652), melanoma (GSE120575), glioblastoma (GSE131928), pilocytic astrocytoma (SCP271), Salmonella stimulation (GSM3855868), and Candida stimulation (eqtlgen.org/candida.html). Given the model's exceptional accuracy in classifying over 100,000 T-cells from new datasets, it was then used to predict T-cell reactivity from the Peng et al. cohort.

Pseudotime analysis of entire tumor microenvironments: The samples were ordered in pseudotime using cell-type specific KEGG pathway scores for the cancer-related or pancreas-related pathways; these were pathways related to cell growth and death, cellular community, the digestive system, the immune system, replication and repair, signal transduction, and cellular transport and catabolism Normalized and scaled cell counts, cancer- and pancreas-related pathway scores, and microbiome abundances for all 35 samples were combined into a single matrix and used as input for SAHMI's pseudotime functions. Normal and tumor states were clustered from the resulting branched dimensionality reduction representation, and the normal state (NS) and tumor state 1 (TS1) were manually split because they completely separated into ends of the same first branch of the pseudotime process. Numerical microbiome and clinical parameters were compared across the tumor states with t-tests, and categorical parameters were compared using Fisher's exact test.

Joint analysis of microbial diversity and survival: The microbiome Shannon diversity index was calculated for each sample in the Peng et al. cohort (Peng et al. Cell Res. 29(9): 725-738, 2019). Patients were stratified by their predicted tumor microbial diversity and the survminer package (github.com/kassambara/survminer/) was used to test the relationship with survival and to plot Kaplan-Meier curves. The relationship between survival and microbial diversity was also tested in TCGA pancreatic cancers using microbial profiles directly estimated from TCGA data by Poore et al (Poore et al. Nature 579: 567-574, 2020). The Shannon diversity index was calculated from TCGA microbiome count data for all genera that passed their quality filters.

Statistical analyses: All statistical analyses were performed using R version 3.6.1. All p-values were false-discovery rate (fdr)- corrected for multiple hypothesis using the p.adjust function with method=“fdr”, unless otherwise stated. The ggpubr package (github.com/kassambara/ggpubr) was used to compare group means with nonparametric tests and to perform multiple hypothesis correction for statistics that are noted in figures. P-values reported as <2.2×10⁻¹⁶ result from reaching the calculation limit for native R statistical test functions and indicate values below this number, not a range of values. Diversity calculations used the vegan package (github.com/vegandevs/vegan).

### Example 2—Results and Discussion

This example describes a particular embodiment of the SAHMI (Single-cell Analysis of Host-Microbiome Interactions) method to examine patterns of human-microbiome interactions in the pancreatic tumor microenvironment at single cell resolution using genomic approaches.

Detection and validation of metagenomic reads in scRNAseq data: Single-cell Analysis of Host-Microbiome Interactions (SAHMI) was developed as a pipeline to reliably identify and annotate metagenomic reads in single-cell RNA sequencing experiments (scRNAseq) and to quantify microbial abundance in human tissue samples. SAHMI enables the systematic assessment of microbial diversity and patterns of microbe-host-cell type interactions at single cell resolution in the tissue microenvironment (FIG. 1A, Example 1), with implications for tissue-level functions and pathological and clinical modalities.

First, SAHMI maps the reads from single cell sequencing experiments to the host genome and uses the resulting transcriptomic signatures to cluster and annotate somatic cell types (Dobin et al. Bioinformatics, 29: 15-21, 2013; Stuart et al. Cell, 177: 1888-1902.e21, 2019). Next, it compares the remaining unmapped reads to a reference microbiome database to detect exact matches, as implemented elsewhere (Wood et al. Genome Biol. 20: 257, 2019), and identifies microbial entities at the most precise taxonomic level possible, estimating their abundance. SAHMI implements a series of filters to remove low quality reads, potentially spurious entries, and laboratory contaminants, only reporting high confidence microbial taxa. The cellular barcodes allow for pairing of microbial entities with corresponding somatic cells at the resolution of single cells. Jointly analyzing the attributes of host cells and associated microbes, SAHMI enables analysis of microbiome and host interactions at multiple levels-from the resolution of individual cells to the level of inter-cellular interactions within the tissue sample microenvironment.

SAHMI was used herein to study tumor-microbiome interactions using scRNAseq data for 24 human pancreatic ductal adenocarcinomas (PDA) and 11 control pancreatic pathologies (non-PDA lesions) (Peng et al. Cell Res. 29(9):725-738, 2019); all samples were obtained during pancreatectomy or pancreatoduodenectomy (Table 1), and all were processed similarly. No batch affects were observed within or between tumor and non-tumor samples (FIG. 6A), mitigating concerns of differential contamination confounding microbiome inferences. These pancreatic tissues had 100-500 million total sequencing reads per sample; after applying multiple quality filters, SAHMI classified 3-10% as bacterial and <1% as fungal (FIG. 6B). SAHMI identified 285 bacterial and 35 fungal genera in PDA and pancreatic tissues, which were detected on 23,546 barcodes, of which 13,848 (58%) also detected RNA from host cells. There was no significant difference in filtered metagenomic read counts between tumor and control samples (FIGS. 6B-6D). However, 68% of microbiome reads from tumor samples were tagged with molecular barcodes which also tagged mRNAs in human somatic cell types, compared to 38% of reads from control samples (Wilcoxon, p=0.001, FIG. 6E). Malignant ductal cells were the cell-types with the highest concentration of metagenomic counts (FIG. 6E). These data indicate broad changes encompassing tissue-microbiome architectural, biochemical, or biophysical properties.

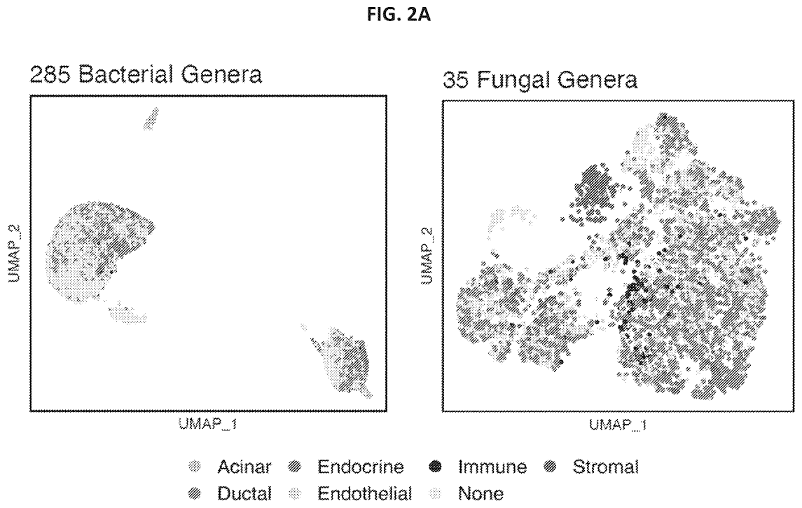

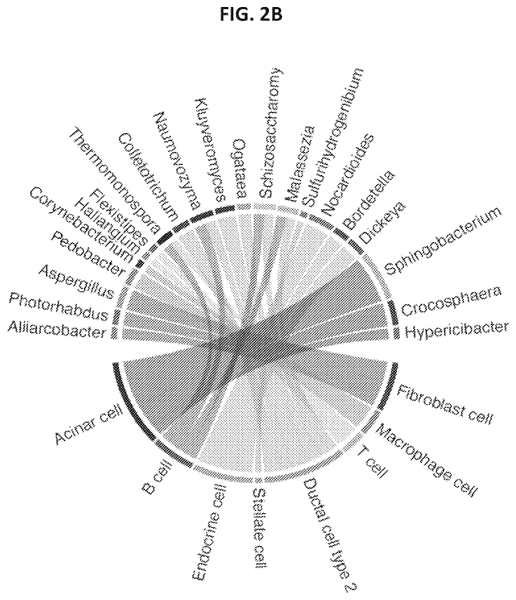

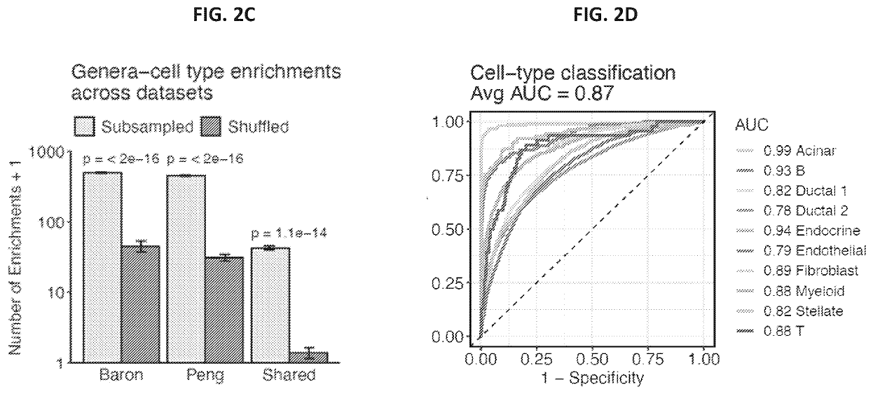

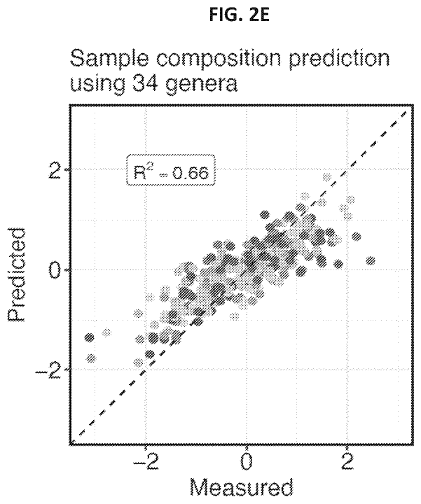

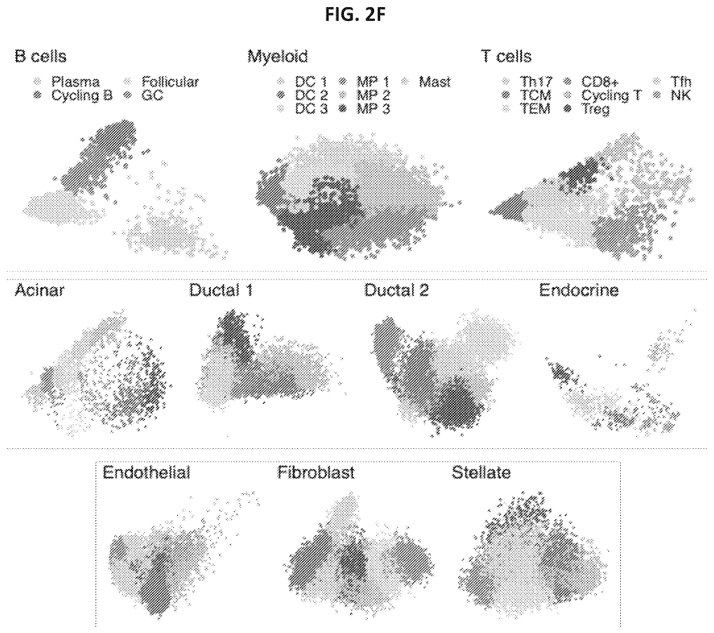

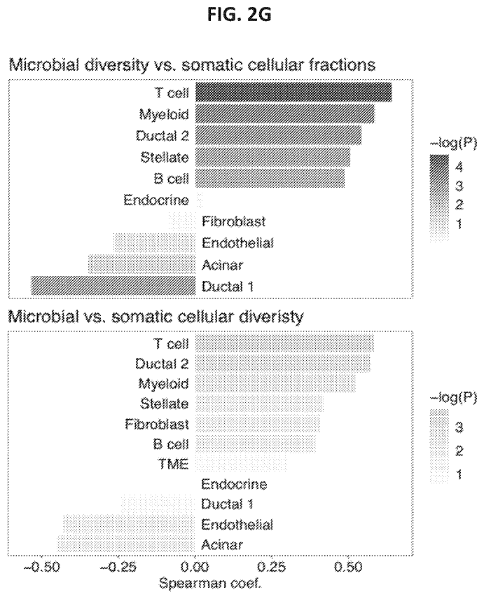

Multiple validation and benchmarking steps were used to ensure that observations were not due to sequencing artifacts or laboratory contamination. First, bacterial entities detected at the genus level from this cohort were compared to (i) entities estimated herein from two other studies that performed single cell sequencing of the normal pancreas (Baron et al. Cell Syst. 3: 346-360.e4, 2016; Muraro et al. Cell Syst. 3: 385-394.e3, 2016), (ii) entities determined from bulk-RNA sequencing data in The Cancer Genome Atlas (TCGA) (Poore et al. Nature, 579: 567-574, 2020), and (iii) entities determined from 16S-rRNA sequencing in a recent large-scale study (Nejman et al. Science, 368(6494): 973-980, 2020)-for a total of 298 pancreatic samples sequenced with three different technologies. Excellent agreement was found, with bacterial compositions showing strong quantitative (mean spearman p=0.61, harmonic mean p-value=9×10⁻⁵², median p=1×10⁻⁵) and qualitative (mean overlap coefficient=0.70) concordance across all datasets (FIG. 1C), with greater consistency across the single-cell studies (p=0.75, harmonic p=4×10⁻⁵²). Next, 20 of 26 prior published differences in bacterial abundances in pancreatic disease samples were detected (Thomas et al. Nat. Rev. Gastroenterol. Hepatol. 17: 53-64, 2020); 19 of the 20 showed significant tumor-normal differences (FIG. 1B; Wilcoxon, p<0.05). The filtered reads were also examined for the putative common laboratory contaminants reported by Poore et al (Poore et al. Nature, 579: 567-574, 2020). Only 19 (9.5%) of 201 detected putative contaminant genera passed the quality filters used herein. All were detected at low expression levels, and 14 of the 19 showed tumor-normal differences (Wilcoxon, p<0.05) (FIG. 1B). Finally, a substantial proportion of the identified microbes were preferentially associated with specific somatic cell types and their cellular activities. Microbiome profiles were also associated with tissue clinical attributes, consistent with collateral literature, as discussed below (FIGS. 2-5), and which cannot be explained by random sequencing artifacts or laboratory contamination. Taken together, these results indicate that SAHMI can reliably quantify microbial abundances from single-cell sequencing data of host tissues at a level comparable to other high-throughput methods, with the advantage of being able to simultaneously analyze somatic cellular gene expression and assess cell-type specific host-microbiome associations.

Pancreatic tumors and non-malignant tissues have distinct microbiomes: Metagenomic data were visualized using uniform manifold approximation and projection (UMAP), a nonlinear dimensionality reduction method that projects the barcode by genus data-table onto a 2-dimensional plane, clustering barcodes with similar metagenomic profiles. The individual bacterial and fungal UMAPs revealed global tumor-normal differences, as indicated by broad separation of tumor and nontumor-derived clusters, as well as multiple barcode clusters with distinct bacterial and fungal compositions (FIG. 1F). Notably, these clusters persisted when data for pancreatic samples from three independent cohorts were jointly analyzed (FIG. 6F), highlighting the consistent detection of a putative commensal microbiome in diverse pancreatic tissues that differs from that of PDAs. Alpha-diversity in the PDA microbiome was significantly increased compared to controls (FIG. 1G).

Specific microbial abundances were then compared between tumor and non-tumor samples using a linear model that includes disease status, total metagenomic counts, and somatic cell counts (to account for selective tropism) as covariates (FIG. 1E, see Methods). Three bacterial genera (Klebsiella spp., Pasteurella spp., Staphylococcus spp.) comprised >80% of the detected microbiome in all the samples from non-malignant illnesses and from most of the tumors (FIG. 1D). A subset of tumors had markedly different microbial compositions, characterized by a decrease in putative commensal genera and an expansion of several low-abundance taxa. These genera included several pathogens previously associated with human infection, with carcinogenesis, or with pancreatic cancer. For example, gut infections by Vibrio spp. (Baker-Austin et al. Nat. Rev. Dis. Prim. 4: 8, 2018) and Campylobacter spp. (Janssen et al. Clin. Microbiol. Rev. 21: 505-518, 2008) are known to cause local and systemic inflammation, Fusobacterium nucleatum is strongly associated with tumorigenesis in colorectal cancer (Sethi et al. Gastroenterology 156: 2097-2115.e2, 2019), Aspergillus spp. produces carcinogenic mycotoxins (Hedayati et al. Microbiology, 153: 1677-1692, 2007), and other taxa, including Prevotella spp., Megamonas spp., Bacteroides spp., Streptococcus spp., Lactobacillus spp., Streptomyces spp., and Clostridium spp. have been associated with pancreatic disease in pre-clinical and epidemiological studies, via differential detection in the oral cavity, plasma, feces, or pancreas (Sethi et al. Gastroenterology, 156: 2097-2115.e2, 2019; Thomas et al. Nat. Rev. Gastroenterol. Hepatol. 17: 53-64, 2020). In total, these findings indicate that pancreatic tumors and non-malignant tissues differ in both microbiome community structure and composition.

Specific host cell-types are enriched with particular microbes: To examine whether bacteria and fungi in human pancreatic tissues are associated with specific host-cell types, barcodes that tagged both metagenomic and somatic RNA were identified. It was observed that metagenomes whose barcodes originated from the same somatic cell-type clustered together in the prior UMAP plots (FIG. 2A), and that specific microbes were significantly enriched in particular cell-types (FIG. 2B). About 500 statistically significant microbiome-host-cell-type enrichments (Table 3) were consistently found in two single-cell pancreas datasets (Peng et al. Cell Res. 29(9):725-738, 2019; Baron et al. Cell Syst. 3: 346-360.e4, 2016), of which ˜50 enrichments were shared across the datasets, which was significantly more than expected by chance when cell-type labels were shuffled (FIG. 2C, Peng: p<2×10⁻¹⁶, Baron: p<2×10⁻¹⁶, Shared: p=1.1×10⁻¹⁴, see Methods). These observations provided further support that the observed microbiome profiles were unlikely to be due to laboratory contaminations or sequencing artifacts, and they suggested the presence of select microbial tropisms with pancreatic cell types. The strongest examples were found between Sphingobacterium spp. and acinar cells (Wilcoxon, p=2e−52) and between Nocardioides spp. and endocrine cells (Wilcoxon, p=4e−26).

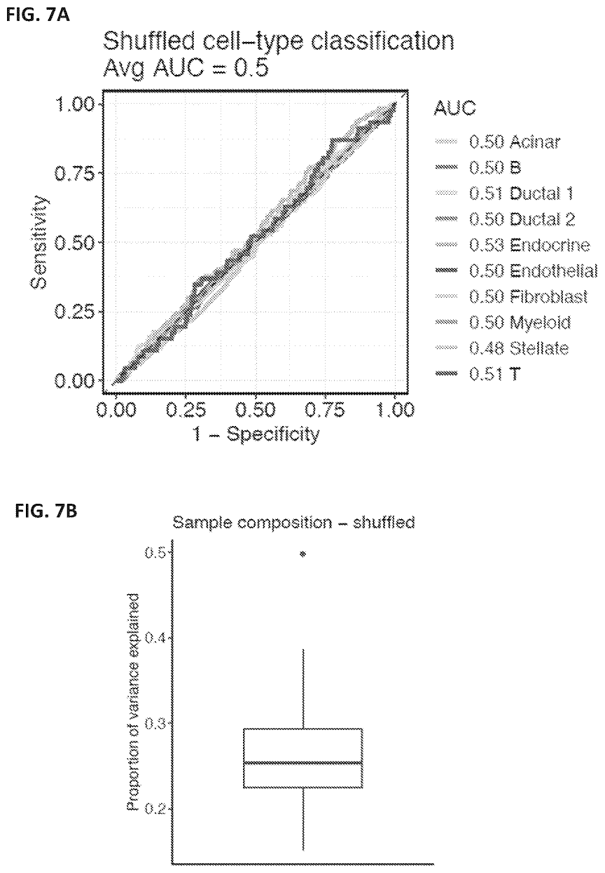

Strong cell type co-localization with particular microbes permitted prediction of barcode cell-types and sample cellular composition based solely on microbiome profiles. A random forest model to predict a barcode's somatic cell-type given its associated metagenomic composition achieved high accuracy in classifying all cell-types (AUC: 0.87; FIG. 2D), and regularized linear regression identified 34 genera whose sample-level abundances accurately predicted somatic cellular composition (r=0.81, FIG. 2E). In contrast, oull models with shuffled sample labels performed poorly (FIGS. 7A-7B). These obeervations indicated tropisms between particular microbes and somatic cells in the pancreas, and provided further validation of microbiome detection from scRNAseq data using SAHMI.

TABLE 3
Cell-type microbiome enrichments.

| Cluster | Genus | P_value | Avg_logFC | Pct. 1 | Pct. 2 | P_val_adj |
|---|---|---|---|---|---|---|
| None | Neisseria | 5.30E−21 | 0.483 | 0.935 | 0.935 | 1.89E−18 |
| None | Granulibacter | 3.93E−11 | 0.636 | 0.490 | 0.282 | 1.40E−08 |
| None | Thalassotalea | 3.81E−06 | 0.302 | 0.710 | 0.580 | 1.36E−03 |
| None | Iodobacter | 1.94E−05 | 0.329 | 0.305 | 0.181 | 6.91E−03 |
| None | Dermabacter | 2.01E−05 | 0.409 | 0.300 | 0.179 | 7.16E−03 |
| Fibroblast | Labilibaculum | 2.34E−21 | 0.753 | 0.680 | 0.421 | 8.32E−19 |
| Fibroblast | Edwardsiella | 1.20E−07 | 0.514 | 0.500 | 0.360 | 4.28E−05 |
| Fibroblast | Kangiella | 1.37E−07 | 0.387 | 0.740 | 0.624 | 4.88E−05 |
| Fibroblast | Solitalea | 2.12E−07 | 0.410 | 0.555 | 0.390 | 7.54E−05 |
| Fibroblast | Yarrowia | 4.47E−07 | 1.497 | 0.290 | 0.170 | 1.59E−04 |
| Fibroblast | Jiangella | 1.72E−06 | 0.343 | 0.410 | 0.270 | 6.11E−04 |
| Fibroblast | Pseudolysobacter | 2.68E−06 | 0.284 | 0.750 | 0.618 | 9.54E−04 |
| Fibroblast | Pochonia | 4.35E−05 | 1.704 | 0.290 | 0.201 | 1.55E−02 |
| Fibroblast | Saccharomyces | 4.59E−05 | 1.687 | 0.290 | 0.200 | 1.63E−02 |
| Fibroblast | Aspergillus | 7.40E−05 | 1.082 | 0.290 | 0.201 | 2.63E−02 |
| Fibroblast | Nakaseomyces | 1.15E−04 | 0.617 | 0.170 | 0.089 | 4.10E−02 |
| Macrophage | Pedobacter | 1.11E−31 | 1.332 | 0.895 | 0.662 | 3.95E−29 |
| Macrophage | Corynebacterium | 1.22E−09 | 0.522 | 0.795 | 0.700 | 4.34E−07 |
| Macrophage | Clostridium | 1.83E−08 | 0.276 | 0.985 | 0.968 | 6.51E−06 |
| Macrophage | Halomonas | 2.36E−08 | 0.480 | 0.885 | 0.854 | 8.39E−06 |
| Macrophage | Xanthomonas | 1.11E−07 | 0.286 | 0.975 | 0.957 | 3.95E−05 |
| Macrophage | Pseudolysobacter | 2.11E−07 | 0.397 | 0.720 | 0.621 | 7.51E−05 |
| Macrophage | Mycoplasma | 3.41E−07 | 0.335 | 0.935 | 0.894 | 1.21E−04 |
| Macrophage | Spiroplasma | 5.80E−07 | 0.260 | 0.900 | 0.809 | 2.06E−04 |
| Macrophage | Bacteroides | 8.84E−07 | 0.516 | 0.760 | 0.685 | 3.15E−04 |
| Macrophage | Campylobacter | 2.79E−06 | 0.263 | 0.950 | 0.905 | 9.93E−04 |
| Macrophage | Acinetobacter | 4.01E−06 | 0.265 | 0.930 | 0.888 | 1.43E−03 |
| Macrophage | Polaribacter | 1.68E−05 | 0.278 | 0.880 | 0.804 | 6.00E−03 |
| Macrophage | Proteus | 2.81E−05 | 0.272 | 0.695 | 0.586 | 1.00E−02 |
| Macrophage | Enterobacter | 4.94E−05 | 0.275 | 0.755 | 0.681 | 1.76E−02 |
| Macrophage | Helicobacter | 9.12E−05 | 0.286 | 0.765 | 0.700 | 3.25E−02 |
| Macrophage | Fusobacterium | 9.97E−05 | 0.296 | 0.925 | 0.906 | 3.55E−02 |
| Macrophage | Calothrix | 1.35E−04 | 0.315 | 0.655 | 0.600 | 4.79E−02 |
| Macrophage | Acetobacter | 1.83E−04 | 0.275 | 0.635 | 0.582 | 6.53E−02 |
| Endothelial | Ilyobacter | 6.51E−10 | 0.383 | 0.435 | 0.230 | 2.32E−07 |
| Endothelial | Rhodoferax | 2.76E−06 | 0.277 | 0.300 | 0.165 | 9.82E−04 |
| Endothelial | Desulfococcus | 5.43E−06 | 0.263 | 0.435 | 0.269 | 1.93E−03 |
| T_cell | Haliangium | 5.39E−18 | 0.556 | 0.842 | 0.714 | 1.92E−15 |
| T_cell | Flexistipes | 7.08E−12 | 0.604 | 0.597 | 0.437 | 2.52E−09 |
| T_cell | Xanthomonas | 9.12E−10 | 0.433 | 0.954 | 0.959 | 3.25E−07 |
| T_cell | Thermomonospora | 7.79E−07 | 0.525 | 0.531 | 0.440 | 2.77E−04 |
| Ductal_2 | Neisseria | 2.13E−17 | 0.411 | 0.970 | 0.932 | 7.59E−15 |
| Ductal_2 | Jiangella | 9.09E−16 | 0.625 | 0.520 | 0.259 | 3.24E−13 |
| Ductal_2 | Kineobactrum | 8.83E−13 | 0.458 | 0.465 | 0.237 | 3.15E−10 |
| Ductal_2 | Ustilago | 8.80E−09 | 0.633 | 0.325 | 0.169 | 3.13E−06 |
| Ductal_2 | Yarrowia | 6.10E−08 | 0.865 | 0.315 | 0.168 | 2.17E−05 |
| Ductal_2 | Pseudolysobacter | 2.13E−07 | 0.410 | 0.780 | 0.615 | 7.58E−05 |
| Ductal_2 | Iodobacter | 2.60E−07 | 0.265 | 0.340 | 0.178 | 9.25E−05 |
| Ductal_2 | Kluyveromyces | 7.89E−07 | 0.846 | 0.305 | 0.166 | 2.81E−04 |
| Ductal_2 | Saccharomyces | 1.30E−06 | 0.790 | 0.330 | 0.196 | 4.64E−04 |
| Ductal_2 | Pochonia | 1.55E−06 | 0.586 | 0.330 | 0.197 | 5.51E−04 |
| Ductal_2 | Pyricularia | 1.67E−06 | 0.362 | 0.325 | 0.184 | 5.96E−04 |
| Ductal_2 | Cryptococcus | 3.71E−06 | 0.326 | 0.330 | 0.196 | 1.32E−03 |
| Ductal_2 | Neurospora | 4.68E−06 | 0.259 | 0.330 | 0.196 | 1.66E−03 |
| Ductal_2 | Zymoseptoria | 5.37E−06 | 0.266 | 0.330 | 0.197 | 1.91E−03 |
| Ductal_2 | Encephalitozoon | 5.73E−06 | 0.650 | 0.330 | 0.194 | 2.04E−03 |
| Ductal_2 | Colletotrichum | 6.37E−06 | 0.503 | 0.330 | 0.197 | 2.27E−03 |
| Ductal_2 | Ogataea | 8.98E−06 | 0.568 | 0.325 | 0.195 | 3.20E−03 |
| Ductal_2 | Fusarium | 9.07E−06 | 0.319 | 0.330 | 0.195 | 3.23E−03 |
| Ductal_2 | Pararhodospirillum | 1.05E−05 | 0.314 | 0.695 | 0.561 | 3.73E−03 |
| Ductal_2 | Thermothielavioides | 1.11E−05 | 0.317 | 0.330 | 0.197 | 3.96E−03 |
| Ductal_2 | Lachancea | 2.08E−05 | 0.455 | 0.205 | 0.104 | 7.40E−03 |
| Ductal_2 | Thermothelomyces | 2.81E−05 | 0.401 | 0.305 | 0.185 | 9.99E−03 |
| Ductal_2 | Sporisorium | 2.91E−05 | 0.496 | 0.325 | 0.196 | 1.04E−02 |
| Ductal_2 | Sugiyamaella | 3.34E−05 | 0.468 | 0.320 | 0.191 | 1.19E−02 |
| Ductal_2 | Eremothecium | 1.11E−04 | 0.357 | 0.225 | 0.125 | 3.96E−02 |
| Stellate | Sulfurihydrogenibium | 3.96E−09 | 0.739 | 0.490 | 0.345 | 1.41E−06 |
| Stellate | Labilibaculum | 5.23E−08 | 0.449 | 0.585 | 0.431 | 1.86E−05 |
| Stellate | Nitrosomonas | 5.10E−07 | 0.431 | 0.380 | 0.249 | 1.82E−04 |
| Stellate | Kangiella | 8.26E−07 | 0.341 | 0.715 | 0.627 | 2.94E−04 |
| Stellate | Xenorhabdus | 6.53E−05 | 0.345 | 0.530 | 0.435 | 2.33E−02 |
| Stellate | Listeria | 7.29E−05 | 0.462 | 0.635 | 0.568 | 2.60E−02 |
| Endocrine | Nocardioides | 3.82E−49 | 1.993 | 0.845 | 0.444 | 1.36E−46 |
| Endocrine | Bordetella | 1.81E−48 | 1.161 | 0.810 | 0.393 | 6.45E−46 |
| Endocrine | Cupriavidus | 3.47E−37 | 0.972 | 0.895 | 0.529 | 1.23E−34 |
| Endocrine | Streptomyces | 1.28E−31 | 1.060 | 1.000 | 0.965 | 4.56E−29 |
| Endocrine | Muricauda | 3.33E−30 | 1.573 | 0.515 | 0.195 | 1.18E−27 |
| Endocrine | Dickeya | 2.20E−29 | 1.387 | 0.810 | 0.433 | 7.82E−27 |
| Endocrine | Hydrogenophaga | 4.51E−29 | 0.950 | 0.735 | 0.434 | 1.60E−26 |
| Endocrine | Pantoea | 8.14E−26 | 0.846 | 0.815 | 0.506 | 2.90E−23 |
| Endocrine | Actinoplanes | 1.36E−25 | 0.904 | 0.675 | 0.338 | 4.85E−23 |
| Endocrine | Hymenobacter | 1.67E−23 | 0.954 | 0.820 | 0.523 | 5.94E−21 |
| Endocrine | Achromobacter | 4.53E−23 | 0.967 | 0.630 | 0.316 | 1.61E−20 |
| Endocrine | Sorangium | 1.63E−18 | 0.899 | 0.635 | 0.349 | 5.79E−16 |
| Endocrine | Nonomuraea | 3.04E−18 | 0.768 | 0.530 | 0.274 | 1.08E−15 |
| Endocrine | Microbacterium | 5.45E−18 | 0.734 | 0.680 | 0.388 | 1.94E−15 |
| Endocrine | Raoultella | 3.56E−17 | 0.503 | 0.460 | 0.194 | 1.27E−14 |
| Endocrine | Chromobacterium | 6.67E−17 | 0.543 | 0.570 | 0.284 | 2.37E−14 |
| Endocrine | Amycolatopsis | 9.97E−17 | 0.734 | 0.590 | 0.313 | 3.55E−14 |
| Endocrine | Deinococcus | 3.07E−16 | 0.774 | 0.735 | 0.465 | 1.09E−13 |
| Endocrine | Micromonospora | 3.37E−16 | 0.927 | 0.835 | 0.611 | 1.20E−13 |
| Endocrine | Pseudolysobacter | 9.39E−16 | 0.449 | 0.870 | 0.606 | 3.34E−13 |
| Endocrine | Mycobacterium | 1.37E−14 | 0.603 | 0.910 | 0.684 | 4.89E−12 |
| Endocrine | Brachybacterium | 1.82E−14 | 0.671 | 0.455 | 0.225 | 6.47E−12 |
| Endocrine | Stenotrophomonas | 1.31E−13 | 0.598 | 0.705 | 0.467 | 4.67E−11 |
| Endocrine | Gordonia | 9.23E−13 | 0.574 | 0.455 | 0.233 | 3.29E−10 |
| Endocrine | Cellulomonas | 1.59E−12 | 0.585 | 0.575 | 0.336 | 5.64E−10 |
| Endocrine | Rathayibacter | 8.97E−12 | 0.750 | 0.455 | 0.253 | 3.19E−09 |
| Endocrine | Methylobacterium | 4.18E−11 | 0.456 | 0.845 | 0.686 | 1.49E−08 |
| Endocrine | Alistipes | 1.28E−10 | 0.644 | 0.335 | 0.166 | 4.56E−08 |
| Endocrine | Nocardia | 3.08E−10 | 0.664 | 0.670 | 0.465 | 1.09E−07 |
| Endocrine | Massilia | 5.28E−10 | 0.501 | 0.540 | 0.327 | 1.88E−07 |
| Endocrine | Rhodococcus | 6.60E−10 | 1.090 | 0.945 | 0.807 | 2.35E−07 |
| Endocrine | Solitalea | 8.45E−10 | 0.309 | 0.615 | 0.384 | 3.01E−07 |
| Endocrine | Frankia | 1.19E−09 | 0.760 | 0.490 | 0.303 | 4.24E−07 |
| Endocrine | Pseudonocardia | 6.48E−09 | 0.361 | 0.470 | 0.270 | 2.31E−06 |
| Endocrine | Actinomyces | 1.12E−08 | 0.617 | 0.635 | 0.447 | 4.00E−06 |
| Endocrine | Bradyrhizobium | 4.27E−08 | 0.722 | 0.630 | 0.461 | 1.52E−05 |
| Endocrine | Desulfovibrio | 7.84E−08 | 0.338 | 0.555 | 0.355 | 2.79E−05 |
| Endocrine | Mycolicibacterium | 1.01E−07 | 0.461 | 0.820 | 0.666 | 3.58E−05 |
| Endocrine | Paraburkholderia | 1.38E−07 | 0.501 | 0.555 | 0.378 | 4.91E−05 |
| Endocrine | Dermabacter | 2.02E−07 | 0.252 | 0.330 | 0.176 | 7.18E−05 |
| Endocrine | Blastochloris | 2.22E−07 | 0.304 | 0.270 | 0.133 | 7.91E−05 |
| Endocrine | Kitasatospora | 2.71E−07 | 0.611 | 0.435 | 0.293 | 9.64E−05 |
| Endocrine | Nocardiopsis | 3.67E−07 | 0.367 | 0.520 | 0.355 | 1.31E−04 |
| Endocrine | Bifidobacterium | 6.42E−07 | 0.391 | 0.825 | 0.651 | 2.29E−04 |
| Endocrine | Granulibacter | 1.10E−06 | 0.289 | 0.460 | 0.285 | 3.91E−04 |
| Endocrine | Myxococcus | 2.50E−06 | 0.469 | 0.460 | 0.315 | 8.88E−04 |
| Endocrine | Geobacillus | 2.56E−05 | 0.833 | 0.380 | 0.266 | 9.12E−03 |
| Endocrine | Bartonella | 8.02E−05 | 0.560 | 0.810 | 0.672 | 2.85E−02 |
| Endocrine | Dokdonia | 9.21E−05 | 0.342 | 0.435 | 0.301 | 3.28E−02 |
| B_cell | Magnetospirillum | 1.51E−25 | 0.741 | 0.795 | 0.568 | 5.37E−23 |
| B_cell | Rhodococcus | 3.76E−25 | 0.504 | 0.885 | 0.813 | 1.34E−22 |
| B_cell | Thermomonospora | 3.26E−23 | 0.667 | 0.715 | 0.422 | 1.16E−20 |
| B_cell | Virgibacillus | 1.35E−21 | 0.510 | 0.900 | 0.767 | 4.79E−19 |
| B_cell | Cercospora | 1.29E−15 | 1.154 | 0.340 | 0.144 | 4.59E−13 |
| B_cell | Ralstonia | 1.86E−14 | 0.269 | 0.960 | 0.941 | 6.62E−12 |
| B_cell | Malassezia | 3.70E−13 | 0.990 | 0.355 | 0.171 | 1.32E−10 |
| B_cell | Debaryomyces | 4.68E−13 | 0.383 | 0.210 | 0.063 | 1.67E−10 |
| B_cell | Naumovozyma | 6.53E−13 | 1.312 | 0.365 | 0.186 | 2.32E−10 |
| B_cell | Eremothecium | 4.93E−12 | 0.675 | 0.295 | 0.118 | 1.76E−09 |
| B_cell | Pyricularia | 4.98E−12 | 0.975 | 0.365 | 0.180 | 1.77E−09 |
| B_cell | Kluyveromyces | 8.13E−12 | 0.535 | 0.355 | 0.161 | 2.90E−09 |
| B_cell | Thermothielavioides | 1.00E−11 | 1.088 | 0.365 | 0.193 | 3.56E−09 |
| B_cell | Colletotrichum | 1.36E−11 | 1.036 | 0.365 | 0.194 | 4.85E−09 |
| B_cell | Schizosaccharomyces | 1.79E−11 | 1.111 | 0.365 | 0.194 | 6.39E−09 |
| B_cell | Sugiyamaella | 3.05E−11 | 0.813 | 0.365 | 0.187 | 1.09E−08 |
| B_cell | Sporisorium | 4.74E−11 | 0.688 | 0.365 | 0.192 | 1.69E−08 |
| B_cell | Torulaspora | 1.14E−10 | 0.273 | 0.175 | 0.055 | 4.07E−08 |
| B_cell | Zygosaccharomyces | 2.42E−10 | 0.452 | 0.210 | 0.076 | 8.60E−08 |
| B_cell | Thermothelomyces | 6.02E−10 | 0.548 | 0.360 | 0.180 | 2.14E−07 |
| B_cell | Fusarium | 6.62E−10 | 0.630 | 0.365 | 0.192 | 2.36E−07 |
| B_cell | Neurospora | 1.08E−09 | 0.770 | 0.365 | 0.192 | 3.84E−07 |
| B_cell | Zymoseptoria | 1.97E−09 | 0.717 | 0.365 | 0.194 | 7.01E−07 |
| B_cell | Cryptococcus | 8.46E−09 | 0.483 | 0.365 | 0.193 | 3.01E−06 |
| B_cell | Ogataea | 3.06E−08 | 0.564 | 0.365 | 0.191 | 1.09E−05 |
| B_cell | Encephalitozoon | 3.33E−08 | 0.597 | 0.360 | 0.191 | 1.19E−05 |
| B_cell | Haliangium | 6.72E−08 | 0.277 | 0.845 | 0.713 | 2.39E−05 |
| B_cell | Lachancea | 1.10E−07 | 0.422 | 0.225 | 0.102 | 3.92E−05 |
| B_cell | Ustilago | 4.83E−07 | 0.460 | 0.315 | 0.170 | 1.72E−04 |
| B_cell | Botrytis | 1.52E−06 | 0.534 | 0.295 | 0.153 | 5.41E−04 |
| B_cell | Thioalkalivibrio | 1.51E−05 | 0.284 | 0.740 | 0.656 | 5.38E−03 |
| Ductal_1 | Neisseria | 3.47E−20 | 0.384 | 0.990 | 0.930 | 1.23E−17 |
| Ductal_1 | Solitalea | 2.24E−09 | 0.407 | 0.595 | 0.386 | 7.98E−07 |
| Acinar | Sphingobacterium | 1.06E−118 | 3.943 | 0.985 | 0.574 | 3.78E−116 |
| Acinar | Pseudolabrys | 3.91E−58 | 0.907 | 0.405 | 0.062 | 1.39E−55 |
| Acinar | Pasteurella | 2.85E−38 | 0.849 | 0.985 | 0.973 | 1.01E−35 |
| Acinar | Crocosphaera | 9.18E−10 | 2.172 | 0.315 | 0.180 | 3.27E−07 |
| Acinar | Thalassotalea | 7.46E−09 | 0.673 | 0.700 | 0.581 | 2.65E−06 |
| Acinar | Nocardia | 1.81E−07 | 0.446 | 0.660 | 0.466 | 6.46E−05 |
| Acinar | Hypericibacter | 2.96E−06 | 0.925 | 0.445 | 0.305 | 1.06E−03 |
| Acinar | Chryseobacterium | 4.71E−06 | 0.276 | 0.830 | 0.927 | 1.68E−03 |

Cluster: cell type cluster; P_val: enrichment p value; Avg_logFC: average log fold change of the genus expression level in the cluster compared to all other clusters; Pct. 1: % of cells in the cluster found with the genus; Pct. 2: % of all other cells found with the genus; P_val_adj: adjusted enrichment p value.

Microbiome diversity correlated with immune cell infiltration and diversity in the microenvironment: Next, the relationship between microbial diversity and tumor cellular composition was assessed. Within the tumor microenvironment (TME), both individual genera and total microbial diversity were significantly associated with abundances of particular somatic cell types, including immune cell infiltrations. Microbial diversity correlated with T-cell infiltration and also with the fraction of myeloid and malignant ductal 2 cells in the tumor. Microbial diversity was strongly negatively correlated with the presence of normal ductal 1 cells (FIG. 2F). Self-assembling manifolds (SAM) (Tarashansky et al. Elife, 8: 1-29, 2019) were then used to identify the major sub-populations within respective cell-types (FIG. 2G). These results indicated that microbial diversity strongly correlated with subpopulation diversity within T-cell, myeloid, and ductal type 2 cells and negatively correlated with diversity within other epithelial and endothelial cell-types (FIG. 2G). The positive correlations with immune and malignant cells suggested that a fraction of the TME immune response may in fact have been responding to local infection, and the negative associations with diversity within typical cells of the pancreas suggested possible phenotypic selection of ‘normal’-like cells within the TME. TME diversity in its totality was only weakly associated with microbial diversity, due to the opposing positive and negative associations (FIG. 2G).

Microbes were associated with specific biological processes in host cells: The microbial abundances that associated with host cell-type specific and sample-level gene expression and pathway activities were examined. The vast majority of microbes and genes or pathways showed no biologically or statistically significant correlations at either the level of the individual host cells or cell-types (FIG. 3B), but a subset showed strong correlations (|r|>0.5, adjusted p<0.05), indicating both known and novel microbiome-physiologic associations (Table 4). These results were analyzed at three levels.

TABLE 4
LASSO coefficients of sample-level microbiota abundances used to predict sample somatic cellular composition.

| | Acinar | B cells | Ductal1 | Ductal2 | Endocrine | Endothelial | Fibroblast | Myeloid | Stellate | T cells |
|---|---|---|---|---|---|---|---|---|---|---|
| Intercept | 0.0000 | 0.0000 | 0.0000 | 0.0000 | 0.0000 | 0.0000 | 0.0000 | 0.0000 | 0.0000 | 0.0000 |
| Aspergillus | −0.0146 | 0.2095 | −0.1373 | 0.2761 | 0.1620 | 0.0767 | 0.4063 | 0.3787 | 0.5688 | 0.4654 |
| Clostridium | −0.0347 | 0.0392 | −0.0443 | 0.0579 | 0.0499 | 0.0222 | 0.0395 | 0.0457 | 0.0818 | 0.0720 |
| Edwardsiella | 0.0032 | −0.0225 | 0.0177 | 0.0557 | −0.0060 | −0.0572 | 0.0161 | 0.0351 | −0.0017 | 0.0243 |
| Flexistipes | 0.0031 | 0.0034 | 0.0026 | −0.0019 | 0.0002 | 0.0034 | −0.0001 | 0.0023 | 0.0020 | 0.0024 |
| Granulibacter | 0.0336 | −0.0315 | 0.0363 | −0.0723 | −0.0075 | −0.0030 | −0.0798 | −0.0454 | −0.0549 | −0.0467 |
| Halanaerobium | −0.0286 | 0.0874 | −0.0309 | 0.1222 | 0.0661 | 0.0070 | 0.0471 | 0.1264 | 0.1360 | 0.1410 |
| Haliangium | −0.0165 | 0.0286 | −0.0498 | 0.0422 | 0.0076 | 0.0010 | −0.0040 | 0.0605 | 0.0154 | 0.0513 |
| Halomonas | 0.0097 | 0.1317 | −0.0361 | −0.0115 | −0.0650 | −0.0065 | −0.0496 | 0.0637 | −0.0190 | 0.0897 |
| Hypericibacter | 0.1030 | 0.0401 | 0.0641 | 0.0458 | 0.0046 | −0.0597 | 0.0597 | 0.0878 | 0.0928 | 0.0340 |
| Iodobacter | −0.2031 | −0.1007 | −0.2025 | 0.1766 | −0.2113 | −0.0838 | −0.1790 | −0.1601 | −0.0930 | −0.0816 |
| Jiangella | −0.1124 | 0.1317 | −0.1533 | 0.1763 | −0.1969 | −0.2574 | 0.0977 | 0.1393 | −0.0065 | 0.1292 |
| Kangiella | 0.0854 | −0.0065 | 0.0770 | −0.0345 | 0.0517 | −0.0236 | 0.0680 | 0.0407 | 0.0501 | −0.0284 |
| Kineobactrum | 0.0019 | 0.0038 | −0.0054 | 0.0059 | −0.0115 | −0.0229 | 0.0051 | 0.0236 | −0.0200 | −0.0019 |
| Kluyveromyces | −0.0211 | 0.0043 | −0.0469 | 0.0490 | −0.0887 | −0.0408 | −0.1416 | −0.0124 | −0.1000 | −0.0145 |
| Komagataella | −0.0115 | −0.0103 | 0.0018 | 0.0065 | −0.0187 | −0.0163 | −0.0406 | −0.0120 | −0.0297 | −0.0093 |
| Labilibaculum | −0.0182 | −0.0401 | 0.0001 | 0.0250 | 0.0647 | 0.0355 | 0.0651 | 0.0276 | 0.0930 | 0.0011 |
| Lachancea | −0.0709 | −0.0338 | −0.0820 | −0.0499 | −0.1721 | −0.1030 | −0.2772 | −0.1085 | −0.1814 | −0.1039 |
| Methylobacterium | −0.0020 | −0.0161 | 0.0011 | 0.0119 | 0.0099 | −0.0257 | 0.0092 | 0.0035 | −0.0039 | −0.0066 |
| Neisseria | 0.0298 | −0.0761 | 0.0404 | 0.0227 | 0.1335 | 0.0594 | 0.0086 | −0.0301 | 0.0078 | −0.0198 |
| Nocardiopsis | −0.1793 | −0.0020 | −0.1817 | 0.1459 | 0.0776 | −0.0715 | −0.0382 | −0.0206 | 0.0238 | 0.0337 |
| Pochonia | −0.0156 | −0.1210 | −0.0100 | 0.0090 | −0.0179 | −0.0970 | −0.0424 | 0.0063 | −0.0696 | −0.0741 |
| Pseudolysobacter | 0.0027 | 0.0063 | −0.0212 | 0.0339 | 0.0155 | −0.0072 | 0.0297 | 0.0562 | 0.0094 | 0.0288 |
| Pseudomonas | −0.0309 | 0.0090 | −0.0216 | −0.0098 | 0.0604 | 0.0204 | 0.0437 | 0.0199 | 0.0357 | 0.0446 |
| Ralstonia | −0.0054 | 0.0155 | −0.0088 | −0.0066 | 0.0085 | 0.0018 | −0.0049 | 0.0045 | 0.0060 | 0.0134 |
| Rhodococcus | 0.0039 | 0.0172 | 0.0057 | −0.0098 | 0.0327 | 0.0359 | −0.0249 | 0.0051 | 0.0196 | 0.0171 |
| Solitalea | 0.1206 | 0.0399 | 0.1188 | −0.1477 | 0.1274 | 0.1377 | 0.0160 | 0.0819 | 0.0534 | −0.0033 |
| Sphingobacterium | 0.3549 | −0.0286 | 0.1362 | −0.0448 | 0.1265 | 0.1957 | −0.0603 | 0.1566 | −0.0585 | −0.0394 |
| Sporisorium | 0.0319 | −0.0015 | 0.0245 | −0.0514 | −0.0660 | −0.0138 | −0.1113 | 0.0138 | −0.0836 | −0.0205 |
| Thermomonospora | −0.0279 | 0.0535 | −0.0278 | 0.0265 | −0.0240 | −0.0187 | −0.0166 | 0.0344 | 0.0101 | 0.0321 |
| Thioalkalivibrio | 0.0531 | 0.0276 | −0.0413 | 0.0622 | 0.0310 | 0.1029 | 0.0647 | 0.0814 | 0.0781 | 0.0015 |
| Virgibacillus | −0.0031 | 0.0060 | −0.0043 | 0.0070 | −0.0011 | 0.0005 | 0.0008 | 0.0043 | 0.0048 | 0.0082 |
| Xanthomonas | −0.0258 | 0.0248 | −0.0266 | 0.0306 | −0.0099 | 0.0137 | −0.0666 | 0.0560 | 0.0250 | 0.0332 |
| Yarrowia | −0.0003 | −0.0015 | 0.0001 | 0.0004 | −0.0004 | −0.0016 | −0.0006 | 0.0001 | −0.0009 | −0.0005 |

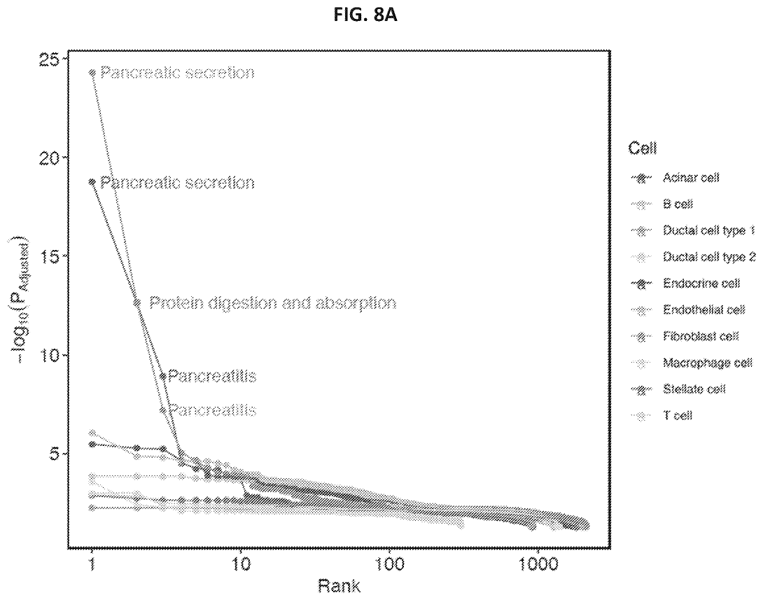

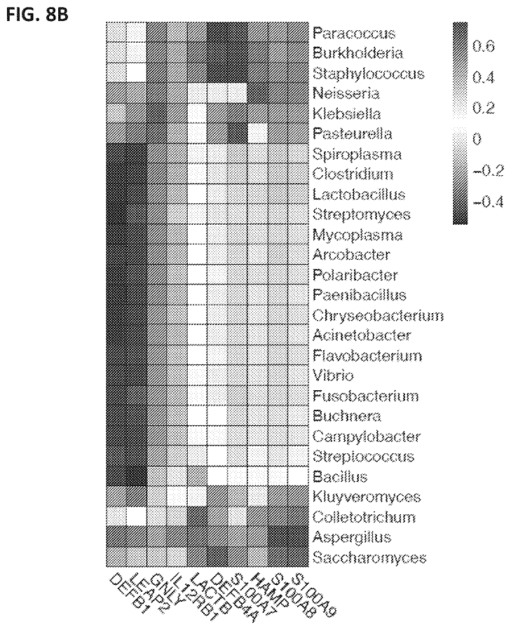

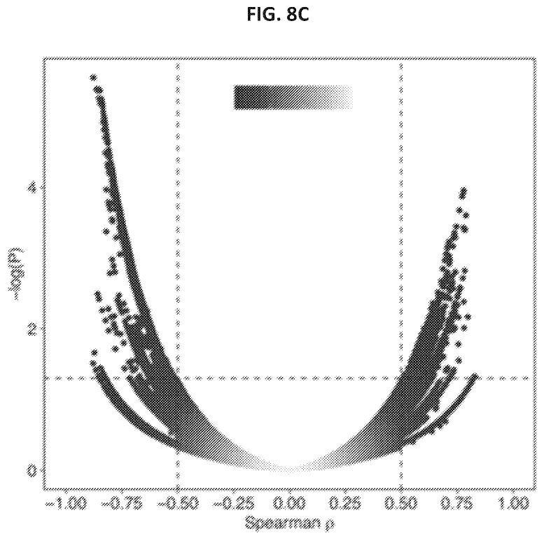

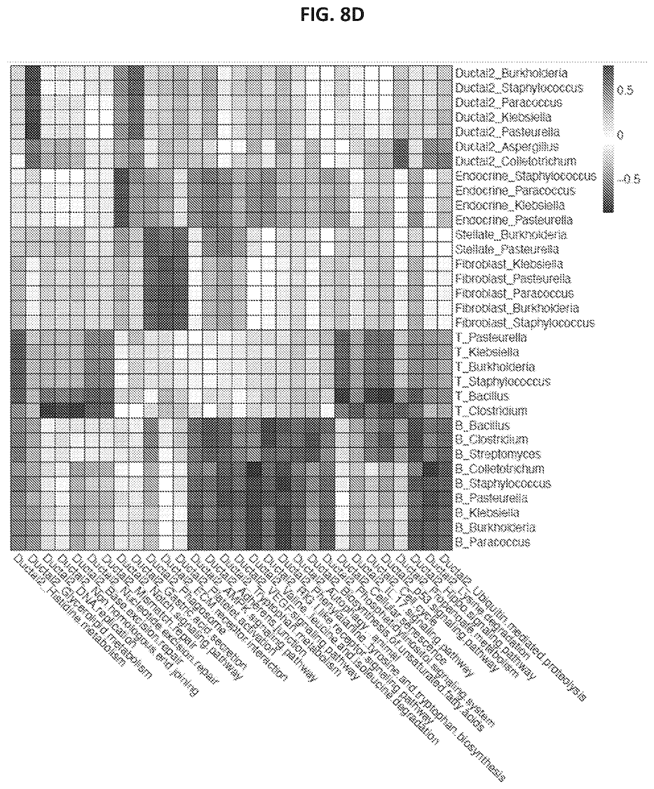

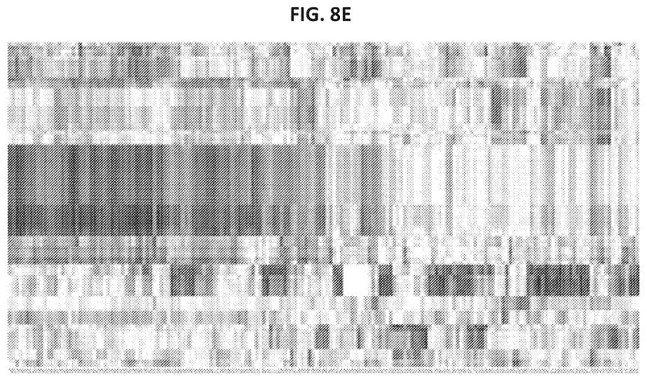

First, interactions between microbiota and receptor gene-expression in their associated host-cell types were examined (FIG. 3A). Expression of particular cell-type specific receptors was strongly associated with the presence of particular microbes in PDA and non-malignant tissues, in largely non-overlapping patterns. In particular, tumor-associated fungi were associated with large groups of receptor expression in T-cells and stellate cells, and these receptors were significantly enriched in pathways for hematopoietic lineage, proteoglycan interactions, the complement cascade, PI3K-AKT signaling, Rap1 signaling, and cell adhesion. Aykut et al. (Aykut et al. Nature, 574: 264-267, 2019) recently showed that pathogenic fungi promote PDA via lectin-induced activation of the complement cascade. The putative commensal bacteria were associated with receptors mostly in acinar and stellate cells that were involved in normal pancreatic functions. Tumor-associated bacteria were strongly associated with receptors involved in PI3K-AKT signaling, adhesion pathways, and cytotoxicity in acinar, endothelial, and T-cells (FIG. 3A). Tumor-associated bacteria also were negatively associated with MET expression in malignant ductal 2 cells and were positively associated with LIFR expression in several cell types, as was recently implicated in PDA pathogenesis (Shi et al. Nature, 569: 131-135, 2019). At the individual cell-level, the microbe-gene expression associations revealed decreases in normal pancreatic secretory activities and increased inflammatory pathways, most strongly in acinar cells and fibroblasts that were rich in profiled microbiome (FIG. 8A).

Second, analysis of microbiome associations with downstream cell-type specific cancer-related pathway activities revealed several known and novel major patterns of interactions (FIGS. 4A-4C). Nearly all tumor-associated bacteria were strongly negatively associated with DNA replication and repair pathways in malignant ductal 2 cells. Infection by Escherichia coli and other microbes can deplete host DNA repair proteins (Sahan et al. Front. Microbiol. 9: 663, 2018; Maddocks et al. MBio. 4: e00152, 2013). Tumor-associated fungi positively correlated with cell cycle, apoptosis, and catabolic pathways in stellate cells, as shown in hepatic stellate cells via Aspergillus-derived gliotoxin (Kweon et al. J. Hepatol. 39: 38-46, 2003). Abundances of a subset of bacteria positively correlated with the PD-1/PD-L1 checkpoint pathway and immune transmigration and with sphingolipid signaling in both immune and endothelial cells, which was consistent with intestinal microbiome influence on anti-PD-1 immunotherapy responses in multiple cancer types (Pushalkar et al. Cancer Discov. 8: 403-416, 2018; Gopalakrishnan et al. Science, 359(6371): 97-103, 2018; Xu et al. Front. Microbiol. 11: 814, 2020). Sphingolipids have been identified as mediators of intestinal-microbiota crosstalk (Bryan et al. Mediators. Inflamm. 2016:9890141, 2016). Microbes also selectively associated with metabolic activities in host cells, including galactose, pentose phosphate, and propanoate metabolism in acinar and T-cells (FIG. 4B). Nearly all bacteria and fungi were associated with increased Hippo signaling in acinar and T-cells, which activates fibroinflammatory programs leading to stromal activation that promotes tumor growth (Liu et al. PLOS Biol. 17: e3000418, 2019; Ansari et al. Anticancer Res. 39: 3317-3321, 2019). At the microenvironment level, particular microbes correlated with inflammatory and antimicrobial gene expression (FIG. 3C, FIG. 8B). Numerous cell-type specific pathway activities correlated with abundances of microbes localized with other cell-types (FIGS. 8C-8D).

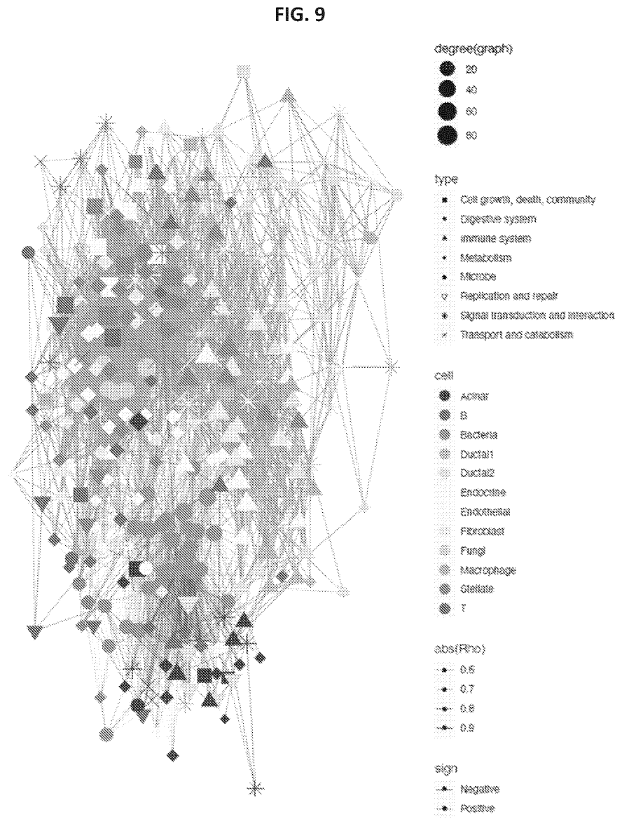

Next, microbe-pathway and cell-specific pathway-pathway interactions were visualized in a network graph, in which the nodes where either microbes or cellular pathways (e.g. T-cell Hippo signaling), and the edges represented significant positive or negative correlations (FIG. 3D, full-size image in FIG. 9). Analysis revealed four major hubs of interactions. Tumor-associated bacteria were closely associated with malignant ductal 2 DNA repair pathways and with acinar and T-cell signaling and metabolism. The other major clusters consisted of tumor microenvironment (TME) growth and metabolic activities, TME immune-related pathways, and ductal 2 specific signaling. Microbes were highly inter-connected in this network and were significantly over-represented in interactions with high edge centrality (FIG. 3E), suggesting that their interactions are common links between multiple TME aspects.

To benchmark these observations, the patterns of microbe-gene/pathway associations detected in our analysis were compared with those inferred from bulk sequencing data in the TCGA pancreatic cancer cohort, and consistent associations were found (FIGS. 3F-3G). For example, strong associations between LYZ expression and Bacteroidetes spp. and between Hippo signaling and Campylobacter spp. were detected in both cohorts. The number of statistically significant microbe-gene/pathway associations that were shared between the two datasets were then compared for both subsampled and label-shuffled data. Analysis indicated significantly more frequent shared associations compared to chance (p<2e−16, FIG. 3H). These observations suggested that microbes are not passive bystanders of tumor progression but may influence key cancer-related cellular processes in individual cell-types in the tumor-microenvironment.

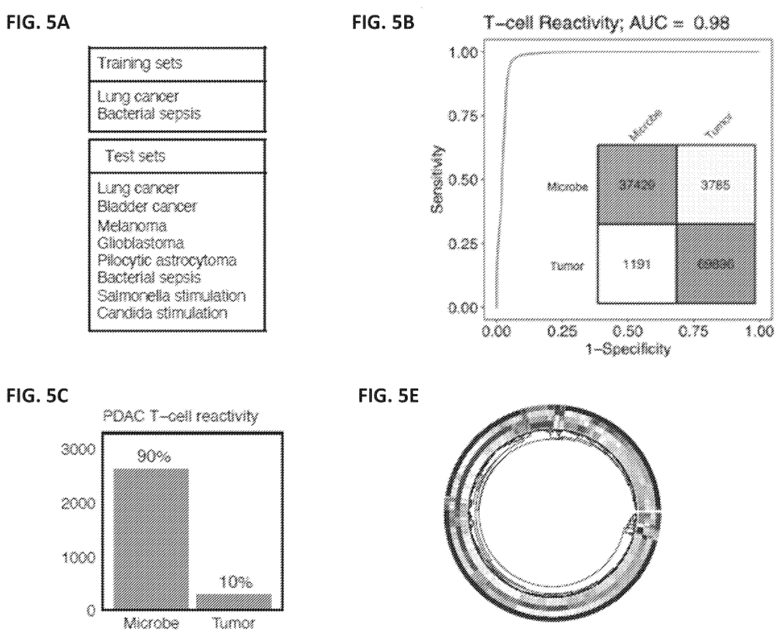

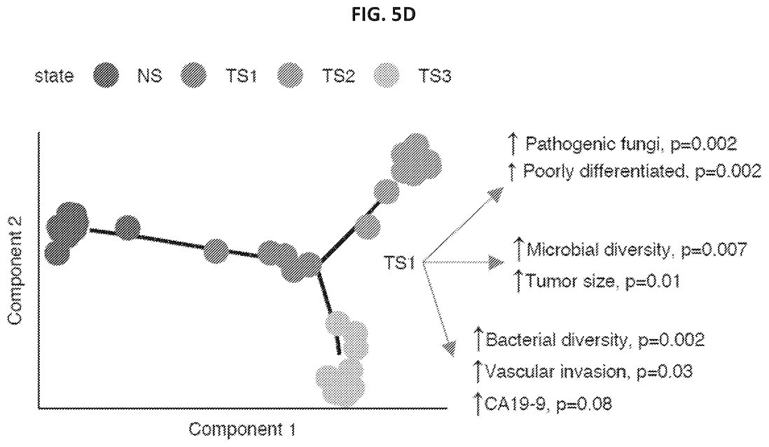

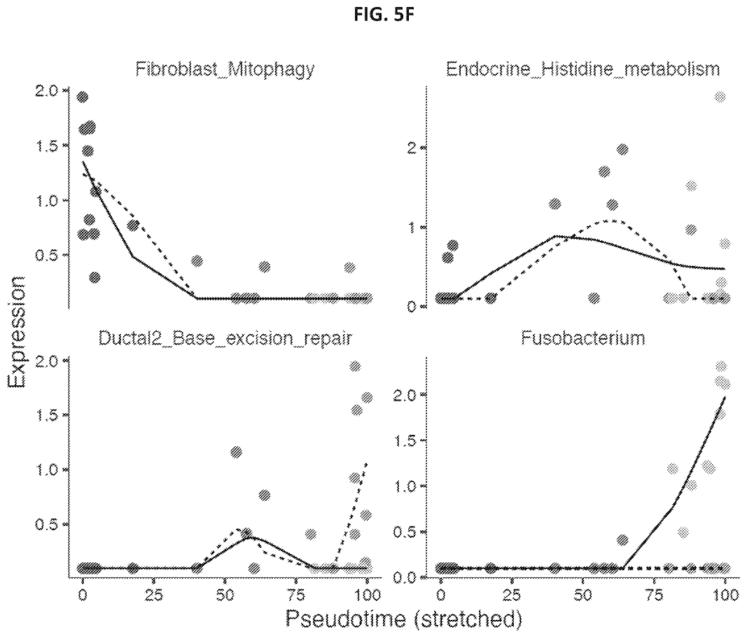

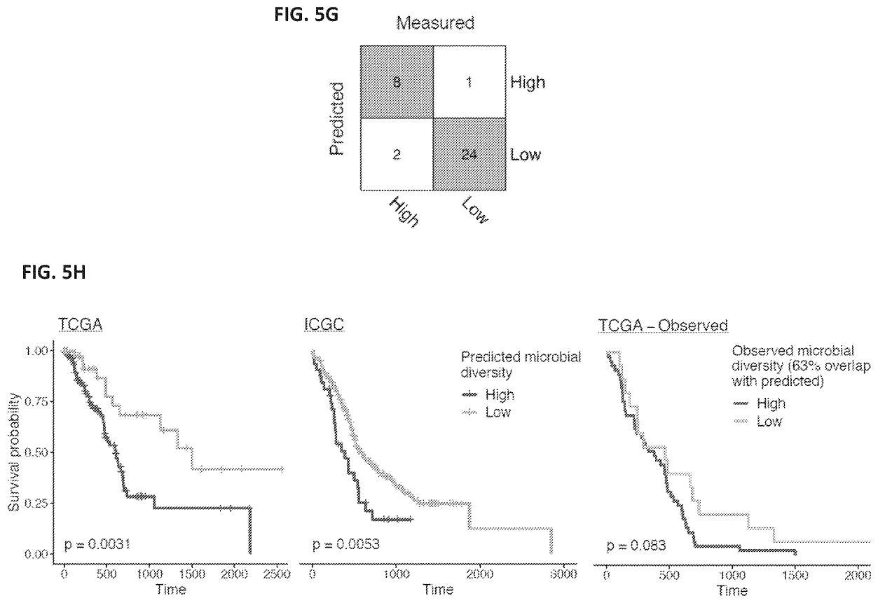

A majority of PDA T-cells were microbe-responsive: In light of the observations that the TME contains Th 17 cells commonly involved in antimicrobial responses (Knochelmann et al. Cell. Mol. Immunol. 15: 458-469, 2018) (FIG. 2F), that microbial diversity correlates with immune cell infiltration and diversity (FIG. 2G), and that particular microbial populations correlate with inflammatory and immune processes (FIGS. 3-4), it was postulated that a fraction of the immune response in the TME is directed against the microbiome and not the malignant T-cells. To test this hypothesis, a random forest model was constructed to distinguish between microbe-reactive and tumor-reactive T-cells based on their gene expression (Methods, FIGS. 5A-5C). First, a model was trained to classify T-cells as either microbe-responding or tumor-responding using T-cells sampled from patients with sepsis and tumors known to have a low microbiome burden (Poore et al. Nature 579: 567-574, 2020; Nejman et al. Science, 368(6494):973-980, 2020). The model was then tested on >100,000 cells taken from each of five cancer types with similarly known low microbiome burden and from three datasets representing either bacterial or fungal infection or stimulation (FIGS. 5A-5B). The model performed exceptionally well in classifying T-cell reactivity, with an AUC of 0.98 (FIG. 5B). Next, this model was used to predict T-cell reactivity in the pancreatic TME. Surprisingly, 90% of the T-cells sequenced in the (Peng et al. Cell Res. 29(9): 725-738, 2019) cohort were classified as microbe-responding.

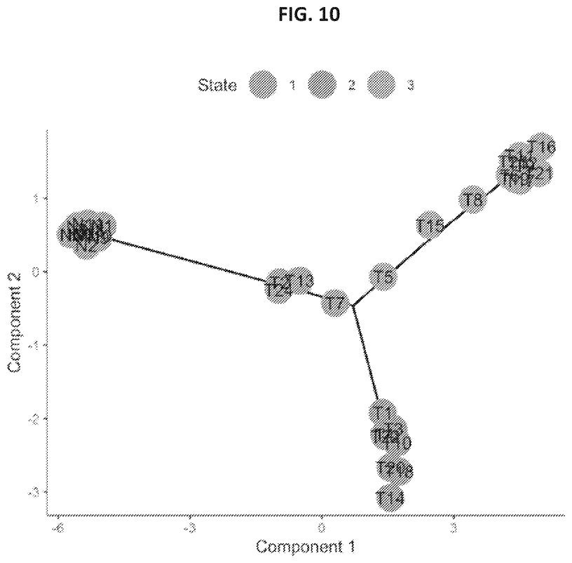

Pseudotime analysis identified tumor-microbiome coevolution and distinct tumor states: To examine how the microbiome might be associated with evolution of the PDA TME, a pseudotime analysis was conducted using Monocle (Trapnell et al. Nat. Biotechnol. 32: 381-386, 2014), which was originally developed for temporal ordering during normal development. TMEs were ordered along a progressive process in a data-driven manner based on their microbiome and cellular activities (FIG. 5D). The results revealed a branching evolutionary process in which pancreatic tissue progressed from a normal state to tumor state 1 (TS1), and then either towards tumor state 2 (TS2), characterized by increased levels of pathogenic fungi (t-test, p=0.002) and poorly differentiated histopathology (Fisher's exact test, p=0.002), or tumor state 3 (TS3), characterized by increased bacterial diversity (t-test, p=0.002), vascular invasion (Fisher's test, p=0.03), and CA19-9 antigen (t-test, p=0.08). Tumor states 2 and 3 were also characterized by a general increase in microbial diversity (t-test, p=0.007) and increased tumor size (t-test, p=0.01). The normal and tumor states had hundreds of significant T-cell-type specific pathway level differences, with the three tumor states clearly distinct from the normal state but retaining state-specific pathway and microbiome signatures (FIGS. 5E-5F, Table 5). For example, TS1 had increased normal ductal 1 arginine biosynthesis, TS2 increased ductal 1 Hippo signaling, and TS3 had decreased DNA repair. These normal and tumor states were observable even when pseudotime analysis was conducted using pathway scores alone, providing further validation of both the microbiome profiles generated herein and their marked relationship to tumor subtype (FIG. 10). Taken together, these results suggest that intra-tumoral microbial dysbiosis is linked with tumor histopathological and clinical attributes and the overall trajectory of tumor evolution.

TABLE 5
Exemplary significant microbe-cell-type specific gene correlations.

| Genus | Gene | Cell | Rho | Padj |
|---|---|---|---|---|
| Acinetobacter | UBD | Acinar | 0.794 | 2.92E−05 |
| Acinetobacter | PODXL | Acinar | 0.788 | 6.23E−05 |
| Acinetobacter | RAB11FIP1 | Acinar | 0.798 | 2.44E−05 |
| Acinetobacter | NNMT | Acinar | 0.770 | 7.18E−05 |
| Acinetobacter | C15orf48 | Acinar | 0.850 | 2.13E−06 |
| Acinetobacter | IL32 | Acinar | 0.812 | 1.38E−05 |
| Acinetobacter | GP2 | Acinar | −0.770 | 7.18E−05 |
| Acinetobacter | CLPS | Acinar | −0.770 | 7.18E−05 |
| Arcobacter | CTSS | Acinar | 0.766 | 3.19E−05 |
| Arcobacter | UBD | Acinar | 0.813 | 4.35E−06 |
| Arcobacter | CFB | Acinar | 0.808 | 5.41E−06 |
| Arcobacter | PODXL | Acinar | 0.823 | 4.54E−06 |
| Arcobacter | RAB11FIP1 | Acinar | 0.825 | 2.32E−06 |
| Arcobacter | RHOD | Acinar | 0.765 | 5.37E−05 |
| Arcobacter | UCP2 | Acinar | 0.817 | 1.96E−05 |
| Arcobacter | NNMT | Acinar | 0.790 | 1.23E−05 |
| Arcobacter | CHPT1 | Acinar | 0.760 | 6.46E−05 |
| Arcobacter | RNASE1 | Acinar | −0.757 | 4.51E−05 |
| Arcobacter | C15orf48 | Acinar | 0.864 | 2.13E−07 |
| Arcobacter | IL32 | Acinar | 0.793 | 1.06E−05 |
| Arcobacter | GP2 | Acinar | −0.775 | 2.26E−05 |
| Arcobacter | INSR | Acinar | 0.783 | 2.70E−05 |
| Arcobacter | NKG7 | Acinar | 0.744 | 7.08E−05 |
| Arcobacter | CLPS | Acinar | −0.782 | 1.71E−05 |
| Arcobacter | CTRL | Acinar | −0.763 | 3.65E−05 |
| Bacillus | UBD | Acinar | 0.795 | 2.75E−05 |
| Bacillus | CFB | Acinar | 0.785 | 4.15E−05 |
| Bacillus | RAB11FIP1 | Acinar | 0.798 | 2.44E−05 |
| Bacillus | FTH1 | Acinar | 0.782 | 4.65E−05 |
| Bacillus | C15orf48 | Acinar | 0.798 | 2.44E−05 |
| Bacillus | GP2 | Acinar | −0.800 | 2.29E−05 |
| Bacteroides | ALCAM | Acinar | 0.793 | 5.13E−05 |
| Bacteroides | SLC12A2 | Acinar | 0.826 | 4.39E−05 |
| Bacteroides | KPNA2 | Acinar | 0.841 | 4.41E−05 |
| Buchnera | TUBB2A | Acinar | 0.831 | 3.61E−05 |
| Buchnera | UBD | Acinar | 0.815 | 1.20E−05 |
| Buchnera | CFB | Acinar | 0.770 | 7.18E−05 |
| Buchnera | PODXL | Acinar | 0.839 | 7.29E−06 |
| Buchnera | RAB11FIP1 | Acinar | 0.880 | 3.21E−07 |
| Buchnera | RARRES3 | Acinar | 0.783 | 4.39E−05 |
| Buchnera | RHOD | Acinar | 0.805 | 3.19E−05 |
| Buchnera | UCP2 | Acinar | 0.867 | 6.67E−06 |
| Buchnera | NNMT | Acinar | 0.824 | 7.95E−06 |
| Buchnera | C15orf48 | Acinar | 0.887 | 1.85E−07 |
| Buchnera | IL32 | Acinar | 0.875 | 4.40E−07 |
| Buchnera | GP2 | Acinar | −0.785 | 4.15E−05 |
| Buchnera | SRCAP | Acinar | 0.782 | 7.52E−05 |
| Buchnera | HN1 | Acinar | 0.805 | 1.90E−05 |
| Buchnera | CLPS | Acinar | −0.824 | 7.95E−06 |
| Buchnera | CTRL | Acinar | −0.803 | 2.02E−05 |
| Campylobacter | F3 | Acinar | 0.794 | 1.01E−05 |
| Campylobacter | CTSS | Acinar | 0.751 | 5.71E−05 |
| Campylobacter | TUBB2A | Acinar | 0.816 | 2.07E−05 |
| Campylobacter | UBD | Acinar | 0.833 | 1.51E−06 |
| Campylobacter | CFB | Acinar | 0.817 | 3.48E−06 |
| Campylobacter | PODXL | Acinar | 0.840 | 1.87E−06 |
| Campylobacter | RAB11FIP1 | Acinar | 0.871 | 1.31E−07 |
| Campylobacter | FTH1 | Acinar | 0.763 | 3.65E−05 |
| Campylobacter | RHOD | Acinar | 0.814 | 7.04E−06 |
| Campylobacter | UCP2 | Acinar | 0.819 | 1.82E−05 |
| Campylobacter | NNMT | Acinar | 0.814 | 4.12E−06 |
| Campylobacter | CHPT1 | Acinar | 0.799 | 1.42E−05 |
| Campylobacter | RNASE1 | Acinar | −0.770 | 2.82E−05 |
| Campylobacter | MEG3 | Acinar | 0.747 | 6.48E−05 |
| Campylobacter | C15orf48 | Acinar | 0.890 | 2.84E−08 |
| Campylobacter | IL32 | Acinar | 0.829 | 1.82E−06 |
| Campylobacter | GP2 | Acinar | −0.816 | 3.68E−06 |
| Campylobacter | SRCAP | Acinar | 0.803 | 1.20E−05 |
| Campylobacter | CLDN7 | Acinar | 0.768 | 4.88E−05 |
| Campylobacter | HN1 | Acinar | 0.748 | 6.23E−05 |
| Campylobacter | INSR | Acinar | 0.799 | 1.42E−05 |
| Campylobacter | CELA3B | Acinar | −0.782 | 1.71E−05 |
| Campylobacter | CLPS | Acinar | −0.774 | 2.36E−05 |
| Campylobacter | CTRL | Acinar | −0.799 | 8.23E−06 |
| Chryseobacterium | CLDN7 | Acinar | 0.800 | 6.78E−05 |
| Clostridium | F3 | Acinar | 0.805 | 3.19E−05 |
| Clostridium | TUBB2A | Acinar | 0.856 | 2.34E−05 |
| Clostridium | UBD | Acinar | 0.802 | 3.66E−05 |
| Clostridium | CFB | Acinar | 0.825 | 1.41E−05 |
| Clostridium | HLA.DRB1 | Acinar | 0.826 | 1.30E−05 |
| Clostridium | SOD2 | Acinar | 0.784 | 7.06E−05 |
| Clostridium | RAB11FIP1 | Acinar | 0.854 | 3.22E−06 |
| Clostridium | FTH1 | Acinar | 0.814 | 2.23E−05 |
| Clostridium | RHOD | Acinar | 0.833 | 1.79E−05 |
| Clostridium | NNMT | Acinar | 0.793 | 5.13E−05 |
| Clostridium | KRT7 | Acinar | 0.793 | 5.13E−05 |
| Clostridium | OLFM4 | Acinar | 0.775 | 9.60E−05 |
| Clostridium | C15orf48 | Acinar | 0.868 | 1.43E−06 |
| Clostridium | IL32 | Acinar | 0.839 | 7.29E−06 |
| Clostridium | FXYD5 | Acinar | 0.777 | 9.04E−05 |
| Clostridium | CELA2B | Acinar | −0.825 | 1.41E−05 |
| Clostridium | AMY2A | Acinar | −0.809 | 2.77E−05 |
| Clostridium | REG3G | Acinar | 0.788 | 6.23E−05 |
| Clostridium | PNLIP | Acinar | −0.791 | 5.47E−05 |
| Clostridium | SYCN | Acinar | −0.825 | 1.41E−05 |
| Flavobacterium | TUBB2A | Acinar | 0.809 | 8.46E−05 |
| Flavobacterium | RAB11FIP1 | Acinar | 0.845 | 2.74E−06 |
| Flavobacterium | RHOD | Acinar | 0.814 | 2.23E−05 |
| Flavobacterium | C15orf48 | Acinar | 0.860 | 1.16E−06 |
| Flavobacterium | IL32 | Acinar | 0.835 | 4.75E−06 |
| Flavobacterium | GP2 | Acinar | −0.765 | 8.40E−05 |
| Flavobacterium | SRCAP | Acinar | 0.802 | 3.66E−05 |
| Flavobacterium | CLDN7 | Acinar | 0.784 | 7.06E−05 |
| Flavobacterium | HN1 | Acinar | 0.771 | 6.81E−05 |
| Flavobacterium | CTRL | Acinar | −0.764 | 8.85E−05 |
| Fusobacterium | F3 | Acinar | 0.765 | 5.37E−05 |
| Fusobacterium | CTSS | Acinar | 0.807 | 9.96E−06 |
| Fusobacterium | DUSP23 | Acinar | 0.770 | 7.18E−05 |
| Fusobacterium | CTSE | Acinar | 0.788 | 3.66E−05 |
| Fusobacterium | TUBB2A | Acinar | 0.853 | 6.68E−06 |
| Fusobacterium | UBD | Acinar | 0.839 | 2.01E−06 |
| Fusobacterium | CFB | Acinar | 0.818 | 5.85E−06 |
| Fusobacterium | PODXL | Acinar | 0.776 | 5.80E−05 |
| Fusobacterium | RAB11FIP1 | Acinar | 0.819 | 5.50E−06 |
| Fusobacterium | PLA2G16 | Acinar | 0.773 | 6.46E−05 |
| Fusobacterium | RHOD | Acinar | 0.798 | 2.44E−05 |
| Fusobacterium | UCP2 | Acinar | 0.840 | 1.26E−05 |
| Fusobacterium | NNMT | Acinar | 0.783 | 2.70E−05 |
| Fusobacterium | CHPT1 | Acinar | 0.780 | 4.91E−05 |
| Fusobacterium | MEG3 | Acinar | 0.804 | 1.13E−05 |
| Fusobacterium | C15orf48 | Acinar | 0.877 | 1.87E−07 |
| Fusobacterium | IL32 | Acinar | 0.800 | 1.34E−05 |
| Fusobacterium | GP2 | Acinar | −0.799 | 1.42E−05 |
| Fusobacterium | CORO1A | Acinar | 0.792 | 9.10E−05 |
| Fusobacterium | NKG7 | Acinar | 0.770 | 4.49E−05 |
| Klebsiella | FTH1 | Acinar | 0.779 | 5.19E−05 |
| Klebsiella | TUBA1B | Acinar | 0.804 | 3.42E−05 |
| Megamonas | TXNRD1 | Acinar | 0.866 | 1.42E−05 |
| Mycoplasma | FBXO2 | Acinar | 0.764 | 8.90E−05 |
| Mycoplasma | RNF186 | Acinar | 0.810 | 8.49E−06 |
| Mycoplasma | CTSS | Acinar | 0.869 | 3.26E−07 |
| Mycoplasma | DUSP23 | Acinar | 0.809 | 1.57E−05 |
| Mycoplasma | CTSE | Acinar | 0.761 | 9.86E−05 |
| Mycoplasma | GNLY | Acinar | 0.795 | 4.74E−05 |
| Mycoplasma | MECOM | Acinar | 0.761 | 9.86E−05 |
| Mycoplasma | TUBB2A | Acinar | 0.802 | 6.28E−05 |
| Mycoplasma | UBD | Acinar | 0.783 | 2.67E−05 |
| Mycoplasma | MEST | Acinar | 0.812 | 8.00E−06 |
| Mycoplasma | DNAJC12 | Acinar | 0.754 | 7.76E−05 |
| Mycoplasma | RHOD | Acinar | 0.783 | 4.39E−05 |
| Mycoplasma | UCP2 | Acinar | 0.850 | 8.05E−06 |
| Mycoplasma | CHPT1 | Acinar | 0.780 | 4.91E−05 |
| Mycoplasma | C15orf48 | Acinar | 0.769 | 4.61E−05 |
| Mycoplasma | HCST | Acinar | 0.764 | 8.87E−05 |
| Mycoplasma | NKG7 | Acinar | 0.827 | 3.73E−06 |
| Paenibacillus | CTSS | Acinar | 0.781 | 8.05E−05 |
| Paenibacillus | SLC12A2 | Acinar | 0.809 | 8.53E−05 |
| Paenibacillus | GP2 | Acinar | −0.782 | 7.66E−05 |
| Pasteurella | TFF1 | Acinar | −0.846 | 1.88E−05 |
| Polaribacter | ITGA2 | Acinar | 0.843 | 1.11E−05 |
| Polaribacter | UCP2 | Acinar | 0.882 | 6.39E−06 |
| Polaribacter | NNMT | Acinar | 0.788 | 6.23E−05 |
| Polaribacter | C15orf48 | Acinar | 0.779 | 8.50E−05 |
| Prevotella | MEST | Acinar | 0.822 | 1.61E−05 |
| Ralstonia | RP11.14N7.2 | Acinar | 0.762 | 5.89E−05 |
| Ralstonia | SOD2 | Acinar | 0.749 | 9.24E−05 |
| Ralstonia | RNASE1 | Acinar | −0.777 | 3.47E−05 |
| Spiroplasma | CTSS | Acinar | 0.851 | 1.04E−06 |
| Spiroplasma | DUSP23 | Acinar | 0.815 | 1.20E−05 |
| Spiroplasma | ALCAM | Acinar | 0.771 | 4.25E−05 |
| Spiroplasma | SLC12A2 | Acinar | 0.835 | 8.71E−06 |
| Spiroplasma | UBD | Acinar | 0.782 | 2.81E−05 |
| Spiroplasma | MAL2 | Acinar | 0.791 | 1.98E−05 |
| Spiroplasma | UCP2 | Acinar | 0.794 | 8.34E−05 |
| Spiroplasma | CHPT1 | Acinar | 0.762 | 9.31E−05 |
| Spiroplasma | C15orf48 | Acinar | 0.770 | 4.40E−05 |
| Spiroplasma | GP2 | Acinar | −0.765 | 5.45E−05 |
| Spiroplasma | SRCAP | Acinar | 0.792 | 3.10E−05 |
| Spiroplasma | INSR | Acinar | 0.764 | 8.85E−05 |
| Spiroplasma | NKG7 | Acinar | 0.757 | 7.09E−05 |
| Staphylococcus | UBD | Acinar | 0.771 | 6.81E−05 |
| Staphylococcus | GSTA1 | Acinar | 0.771 | 6.81E−05 |
| Staphylococcus | FTH1 | Acinar | 0.812 | 1.38E−05 |
| Staphylococcus | RHOD | Acinar | 0.795 | 4.80E−05 |
| Staphylococcus | TUBA1B | Acinar | 0.779 | 8.50E−05 |
| Staphylococcus | CELA2B | Acinar | −0.765 | 8.40E−05 |
| Staphylococcus | AMY2A | Acinar | −0.800 | 2.29E−05 |
| Staphylococcus | PNLIP | Acinar | −0.761 | 9.80E−05 |
| Staphylococcus | CTRL | Acinar | −0.800 | 2.29E−05 |
| Streptococcus | TUBB2A | Acinar | 0.811 | 7.74E−05 |
| Streptococcus | UBD | Acinar | 0.795 | 2.75E−05 |
| Streptococcus | CFB | Acinar | 0.795 | 2.75E−05 |
| Streptococcus | PODXL | Acinar | 0.811 | 2.58E−05 |
| Streptococcus | RAB11FIP1 | Acinar | 0.823 | 8.53E−06 |
| Streptococcus | RHOD | Acinar | 0.777 | 9.04E−05 |
| Streptococcus | NNMT | Acinar | 0.802 | 2.15E−05 |
| Streptococcus | RNASE1 | Acinar | −0.776 | 5.80E−05 |
| Streptococcus | C15orf48 | Acinar | 0.863 | 9.62E−07 |
| Streptococcus | IL32 | Acinar | 0.818 | 1.05E−05 |
| Streptococcus | GP2 | Acinar | −0.789 | 3.49E−05 |
| Streptomyces | CTSS | Acinar | 0.786 | 2.43E−05 |
| Streptomyces | DUSP23 | Acinar | 0.795 | 1.67E−05 |
| Streptomyces | CPB1 | Acinar | −0.755 | 7.74E−05 |
| Streptomyces | UBD | Acinar | 0.855 | 8.16E−07 |
| Streptomyces | CFB | Acinar | 0.827 | 3.73E−06 |
| Streptomyces | GSTA1 | Acinar | 0.813 | 7.49E−06 |
| Streptomyces | SOD2 | Acinar | 0.788 | 2.19E−05 |
| Streptomyces | PODXL | Acinar | 0.791 | 3.29E−05 |
| Streptomyces | RAB11FIP1 | Acinar | 0.822 | 4.84E−06 |
| Streptomyces | EIF4EBP1 | Acinar | 0.749 | 9.24E−05 |
| Streptomyces | FTH1 | Acinar | 0.826 | 3.99E−06 |
| Streptomyces | PLA2G16 | Acinar | 0.798 | 2.44E−05 |
| Streptomyces | UCP2 | Acinar | 0.806 | 5.38E−05 |
| Streptomyces | NNMT | Acinar | 0.801 | 1.27E−05 |
| Streptomyces | KRT7 | Acinar | 0.799 | 1.42E−05 |
| Streptomyces | CHPT1 | Acinar | 0.815 | 1.20E−05 |
| Streptomyces | OLFM4 | Acinar | 0.825 | 4.26E−06 |
| Streptomyces | MEG3 | Acinar | 0.773 | 4.03E−05 |
| Streptomyces | C15orf48 | Acinar | 0.879 | 1.54E−07 |
| Streptomyces | IL32 | Acinar | 0.779 | 3.14E−05 |
| Streptomyces | GP2 | Acinar | −0.805 | 1.07E−05 |
| Streptomyces | SRCAP | Acinar | 0.785 | 4.15E−05 |
| Streptomyces | SDC4 | Acinar | 0.773 | 4.03E−05 |
| Streptomyces | WFDC2 | Acinar | 0.770 | 7.18E−05 |
| Streptomyces | INSR | Acinar | 0.829 | 6.40E−06 |
| Streptomyces | C19orf33 | Acinar | 0.753 | 8.10E−05 |
| Streptomyces | RPS16 | Acinar | 0.764 | 5.62E−05 |
| Streptomyces | CELA3B | Acinar | −0.781 | 2.99E−05 |
| Streptomyces | CELA3A | Acinar | −0.789 | 3.49E−05 |
| Streptomyces | AMY2A | Acinar | −0.758 | 6.76E−05 |
| Streptomyces | CLPS | Acinar | −0.771 | 4.23E−05 |
| Streptomyces | CTRL | Acinar | −0.757 | 7.08E−05 |
| Streptomyces | CTRB1 | Acinar | −0.762 | 5.89E−05 |
| Streptomyces | SYCN | Acinar | −0.749 | 9.24E−05 |
| Vibrio | FBXO2 | Acinar | 0.812 | 2.41E−05 |
| Vibrio | CTSS | Acinar | 0.828 | 6.44E−06 |
| Vibrio | DUSP23 | Acinar | 0.777 | 9.04E−05 |
| Vibrio | MECOM | Acinar | 0.781 | 8.05E−05 |
| Vibrio | UBD | Acinar | 0.763 | 9.22E−05 |
| Vibrio | RHOD | Acinar | 0.795 | 4.80E−05 |
| Vibrio | UCP2 | Acinar | 0.809 | 8.31E−05 |
| Vibrio | PMAIP1 | Acinar | 0.784 | 7.24E−05 |
| Megamonas | PLK1 | B_cell | −0.939 | 5.62E−05 |
| Sphingobacterium | KIF2C | B_cell | −0.918 | 6.80E−05 |
| Sphingobacterium | CENPE | B_cell | −0.918 | 6.80E−05 |
| Sphingobacterium | KIFC1 | B_cell | −0.922 | 5.29E−05 |
| Sphingobacterium | SCG5 | B_cell | −0.924 | 4.78E−05 |
| Sphingobacterium | UBE2C | B_cell | −0.925 | 4.59E−05 |
| Aspergillus | SCTR | B_cell | −0.942 | 4.54E−05 |
| Colletotrichum | SCTR | B_cell | −0.930 | 9.60E−05 |
| Acinetobacter | CYR61 | Ductal1 | −0.675 | 2.24E−05 |
| Acinetobacter | S100A6 | Ductal1 | 0.627 | 9.55E−05 |
| Acinetobacter | TAGLN3 | Ductal1 | −0.700 | 3.43E−05 |
| Acinetobacter | MMP7 | Ductal1 | 0.632 | 7.88E−05 |
| Acinetobacter | ADCYAP1 | Ductal1 | −0.697 | 2.70E−05 |
| Acinetobacter | FOSB | Ductal1 | −0.653 | 3.73E−05 |
| Acinetobacter | CTRL | Ductal1 | −0.651 | 7.20E−05 |
| Campylobacter | CUZD1 | Ductal1 | −0.673 | 4.57E−05 |
| Campylobacter | MDK | Ductal1 | 0.678 | 3.80E−05 |
| Campylobacter | PCDH17 | Ductal1 | −0.702 | 1.53E−05 |
| Campylobacter | CTRB1 | Ductal1 | −0.680 | 3.58E−05 |
| Chryseobacterium | TAGLN3 | Ductal1 | −0.725 | 4.18E−05 |
| Chryseobacterium | MDK | Ductal1 | 0.683 | 3.17E−05 |
| Chryseobacterium | LINC00261 | Ductal1 | −0.664 | 8.48E−05 |
| Clostridium | MDK | Ductal1 | 0.674 | 4.46E−05 |
| Fusobacterium | TAGLN3 | Ductal1 | −0.724 | 4.34E−05 |
| Megamonas | CD2 | Ductal1 | 0.854 | 1.45E−08 |
| Megamonas | CAPN8 | Ductal1 | 0.701 | 6.66E−05 |
| Megamonas | IL7R | Ductal1 | 0.754 | 8.50E−06 |
| Megamonas | LST1 | Ductal1 | 0.707 | 3.79E−05 |
| Megamonas | FAM26F | Ductal1 | 0.758 | 2.93E−06 |
| Megamonas | AZGP1 | Ductal1 | 0.716 | 5.61E−05 |
| Megamonas | FAM214B | Ductal1 | 0.745 | 8.42E−06 |
| Megamonas | CHRDL2 | Ductal1 | 0.719 | 3.46E−05 |
| Megamonas | VSIG2 | Ductal1 | 0.726 | 3.96E−05 |
| Megamonas | MSLN | Ductal1 | 0.723 | 6.68E−05 |
| Megamonas | MAFB | Ductal1 | 0.753 | 5.78E−06 |
| Megamonas | C19orf77 | Ductal1 | 0.801 | 8.98E−07 |
| Megamonas | CEACAM6 | Ductal1 | 0.733 | 1.38E−05 |
| Megamonas | TFF3 | Ductal1 | 0.759 | 6.97E−06 |
| Paenibacillus | GRB7 | Ductal1 | −0.703 | 8.94E−05 |
| Polaribacter | LINC00261 | Ductal1 | −0.663 | 8.91E−05 |
| Prevotella | RP11.528G1.2 | Ductal1 | −0.689 | 1.82E−05 |
| Prevotella | HLA.DRB1 | Ductal1 | 0.691 | 1.67E−05 |
| Prevotella | HLA.DPA1 | Ductal1 | 0.656 | 6.15E−05 |
| Prevotella | MDK | Ductal1 | 0.671 | 3.66E−05 |
| Prevotella | MMP7 | Ductal1 | 0.662 | 4.97E−05 |
| Prevotella | LYZ | Ductal1 | 0.686 | 2.02E−05 |
| Prevotella | PCDH17 | Ductal1 | −0.700 | 1.16E−05 |
| Prevotella | HSD17B2 | Ductal1 | 0.769 | 4.44E−07 |
| Prevotella | KRT19 | Ductal1 | 0.686 | 2.06E−05 |
| Prevotella | CLPS | Ductal1 | −0.643 | 9.63E−05 |
| Prevotella | CTRB1 | Ductal1 | −0.689 | 1.85E−05 |
| Prevotella | SNORD3D | Ductal1 | −0.653 | 9.22E−05 |
| Spiroplasma | ERO1LB | Ductal1 | −0.723 | 4.32E−06 |
| Aspergillus | HSPD1 | Ductal2 | 0.729 | 7.89E−05 |
| Aspergillus | ZFAND2A | Ductal2 | 0.748 | 4.06E−05 |
| Aspergillus | LDHA | Ductal2 | 0.725 | 9.01E−05 |
| Colletotrichum | HSPD1 | Ductal2 | 0.765 | 2.14E−05 |
| Colletotrichum | ZFAND2A | Ductal2 | 0.746 | 4.37E−05 |
| Colletotrichum | LDHA | Ductal2 | 0.786 | 8.94E−06 |
| Colletotrichum | RHOD | Ductal2 | 0.732 | 7.13E−05 |
| Saccharomyces | ZFAND2A | Ductal2 | 0.799 | 4.74E−06 |
| Saccharomyces | LDHA | Ductal2 | 0.792 | 6.85E−06 |
| Saccharomyces | RHOD | Ductal2 | 0.749 | 3.92E−05 |
| Thermothielavioides | HSPD1 | Ductal2 | 0.737 | 6.01E−05 |
| Thermothielavioides | ZFAND2A | Ductal2 | 0.779 | 1.21E−05 |
| Thermothielavioides | LDHA | Ductal2 | 0.781 | 1.11E−05 |
| Thermothielavioides | RHOD | Ductal2 | 0.753 | 3.38E−05 |
| Campylobacter | PDPN | Endocrine | −0.754 | 5.13E−05 |
| Megamonas | AMN | Endocrine | 0.704 | 8.54E−05 |
| Megamonas | BIK | Endocrine | 0.727 | 1.78E−05 |
| Pasteurella | TMEM97 | Endocrine | 0.760 | 4.12E−05 |
| Spiroplasma | TCN1 | Endocrine | 0.684 | 8.30E−05 |
| Staphylococcus | C10orf10 | Endocrine | 0.760 | 6.46E−05 |
| Aspergillus | LINC01133 | Endocrine | 0.725 | 9.14E−05 |
| Aspergillus | FMO3 | Endocrine | 0.741 | 7.91E−05 |
| Aspergillus | CD8A | Endocrine | 0.691 | 9.21E−05 |
| Aspergillus | TNNC1 | Endocrine | 0.758 | 7.28E−06 |
| Aspergillus | CITED1 | Endocrine | 0.761 | 3.96E−05 |
| Aspergillus | LCN6.1 | Endocrine | 0.769 | 1.13E−05 |
| Aspergillus | NKX2.3 | Endocrine | 0.717 | 5.51E−05 |
| Aspergillus | CLEC14A | Endocrine | 0.710 | 4.78E−05 |
| Aspergillus | WFDC1 | Endocrine | 0.818 | 3.25E−06 |
| Aspergillus | ADAMTS5 | Endocrine | 0.731 | 7.34E−05 |
| Colletotrichum | CD8A | Endocrine | 0.744 | 2.03E−05 |
| Colletotrichum | ACKR3 | Endocrine | 0.750 | 9.04E−05 |
| Colletotrichum | TNNC1 | Endocrine | 0.718 | 5.40E−05 |
| Colletotrichum | AK8 | Endocrine | 0.769 | 2.84E−05 |
| Colletotrichum | LCN6.1 | Endocrine | 0.772 | 1.61E−05 |
| Colletotrichum | WFDC1 | Endocrine | 0.855 | 8.06E−07 |
| Colletotrichum | ADAMTS5 | Endocrine | 0.738 | 8.84E−05 |
| Kluyveromyces | ALPL | Endocrine | 0.828 | 1.20E−05 |
| Kluyveromyces | FMO3 | Endocrine | 0.735 | 9.84E−05 |
| Kluyveromyces | TNNC1 | Endocrine | 0.804 | 2.24E−06 |
| Kluyveromyces | MYCT1 | Endocrine | 0.828 | 1.20E−05 |
| Kluyveromyces | IL3RA | Endocrine | 0.794 | 1.04E−05 |
| Kluyveromyces | CITED1 | Endocrine | 0.784 | 2.64E−05 |
| Kluyveromyces | GPIHBP1 | Endocrine | 0.980 | 1.01E−12 |
| Kluyveromyces | IL33 | Endocrine | 0.892 | 1.30E−07 |
| Kluyveromyces | LCN6.1 | Endocrine | 0.735 | 9.65E−05 |
| Kluyveromyces | MRC1 | Endocrine | 0.810 | 1.51E−05 |
| Kluyveromyces | KLRC2 | Endocrine | 0.775 | 9.76E−05 |
| Kluyveromyces | KRT86 | Endocrine | 0.804 | 1.92E−05 |
| Kluyveromyces | RP11.841O20.2 | Endocrine | 0.790 | 3.47E−05 |
| Kluyveromyces | WFDC1 | Endocrine | 0.756 | 7.27E−05 |
| Saccharomyces | LINC01133 | Endocrine | 0.749 | 3.89E−05 |
| Saccharomyces | CD8A | Endocrine | 0.697 | 7.64E−05 |
| Saccharomyces | ACKR3 | Endocrine | 0.738 | 8.70E−05 |
| Saccharomyces | TNNC1 | Endocrine | 0.761 | 6.41E−06 |
| Saccharomyces | CITED1 | Endocrine | 0.793 | 1.08E−05 |
| Saccharomyces | LCN6.1 | Endocrine | 0.755 | 2.00E−05 |
| Saccharomyces | NKX2.3 | Endocrine | 0.733 | 3.12E−05 |
| Saccharomyces | CLEC14A | Endocrine | 0.710 | 4.91E−05 |
| Saccharomyces | WFDC1 | Endocrine | 0.817 | 3.48E−06 |
| Saccharomyces | ADAMTS5 | Endocrine | 0.754 | 3.26E−05 |
| Thermothielavioides | LINC01133 | Endocrine | 0.757 | 2.91E−05 |
| Thermothielavioides | CD8A | Endocrine | 0.693 | 8.73E−05 |
| Thermothielavioides | TNNC1 | Endocrine | 0.742 | 1.44E−05 |
| Thermothielavioides | CITED1 | Endocrine | 0.747 | 6.50E−05 |
| Thermothielavioides | LCN6.1 | Endocrine | 0.764 | 1.40E−05 |
| Thermothielavioides | NKX2.3 | Endocrine | 0.711 | 6.68E−05 |
| Thermothielavioides | CLEC14A | Endocrine | 0.720 | 3.40E−05 |
| Thermothielavioides | WFDC1 | Endocrine | 0.820 | 3.03E−06 |
| Thermothielavioides | ADAMTS5 | Endocrine | 0.731 | 7.34E−05 |
| Arcobacter | CD2 | Endothelial | 0.656 | 6.22E−05 |
| Arcobacter | DNAJC12 | Endothelial | 0.669 | 5.38E−05 |
| Arcobacter | KCNN4 | Endothelial | 0.702 | 1.10E−05 |
| Bacteroides | CD53 | Endothelial | 0.667 | 7.90E−05 |
| Bacteroides | HIST2H2AA3 | Endothelial | 0.689 | 7.00E−05 |
| Bacteroides | MNDA | Endothelial | 0.700 | 4.85E−05 |
| Bacteroides | FCGR2B | Endothelial | 0.682 | 4.54E−05 |
| Bacteroides | SLC11A1 | Endothelial | 0.716 | 1.85E−05 |
| Bacteroides | CXCL5 | Endothelial | 0.705 | 8.28E−05 |
| Bacteroides | CSF2RA | Endothelial | 0.701 | 6.71E−05 |
| Bacteroides | SPI1 | Endothelial | 0.674 | 8.42E−05 |
| Bacteroides | TCN1 | Endothelial | 0.689 | 5.00E−05 |
| Bacteroides | PTPRCAP | Endothelial | 0.692 | 3.25E−05 |
| Bacteroides | AMICA1 | Endothelial | 0.722 | 9.82E−06 |
| Bacteroides | CD3D | Endothelial | 0.725 | 8.64E−06 |
| Bacteroides | RNASE6 | Endothelial | 0.687 | 7.66E−05 |
| Bacteroides | BATF | Endothelial | 0.749 | 3.01E−06 |
| Bacteroides | LIMD2 | Endothelial | 0.696 | 3.88E−05 |
| Bacteroides | CD7 | Endothelial | 0.720 | 1.08E−05 |
| Bacteroides | CST7 | Endothelial | 0.660 | 9.67E−05 |
| Bacteroides | HCST | Endothelial | 0.731 | 6.64E−06 |
| Bacteroides | KCNN4 | Endothelial | 0.707 | 1.82E−05 |
| Bacteroides | RAC2 | Endothelial | 0.688 | 3.78E−05 |
| Bacteroides | LGALS1 | Endothelial | 0.695 | 8.13E−05 |
| Bacteroides | ITGB2 | Endothelial | 0.689 | 3.58E−05 |
| Burkholderia | NOX5 | Endothelial | −0.676 | 5.66E−05 |
| Chryseobacterium | CCND1 | Endothelial | −0.666 | 1.27E−05 |
| Chryseobacterium | PLXDC1 | Endothelial | 0.630 | 4.93E−05 |
| Clostridium | CXCL5 | Endothelial | 0.706 | 3.92E−05 |
| Clostridium | KCNN4 | Endothelial | 0.651 | 9.65E−05 |
| Flavobacterium | GPAT2 | Endothelial | 0.660 | 7.23E−05 |
| Flavobacterium | CCND1 | Endothelial | −0.689 | 2.55E−05 |
| Fusobacterium | CENPW | Endothelial | 0.656 | 6.24E−05 |
| Fusobacterium | CCND1 | Endothelial | −0.652 | 2.19E−05 |
| Fusobacterium | PLXDC1 | Endothelial | 0.633 | 4.54E−05 |
| Fusobacterium | KCNN4 | Endothelial | 0.665 | 1.75E−05 |
| Megamonas | CD8A | Endothelial | 0.737 | 7.69E−06 |
| Megamonas | COL7A1 | Endothelial | 0.693 | 4.29E−05 |
| Megamonas | EREG | Endothelial | 0.720 | 3.32E−05 |
| Megamonas | CYBB | Endothelial | 0.727 | 2.57E−05 |
| Megamonas | BATF | Endothelial | 0.670 | 7.00E−05 |
| Mycoplasma | CXCL5 | Endothelial | 0.675 | 8.07E−05 |
| Mycoplasma | DNAJC12 | Endothelial | 0.733 | 4.14E−06 |
| Mycoplasma | KCNN4 | Endothelial | 0.695 | 1.45E−05 |
| Paenibacillus | CD3D | Endothelial | 0.658 | 7.84E−05 |
| Paracoccus | NOX5 | Endothelial | −0.726 | 8.36E−06 |
| Spiroplasma | CADM3 | Endothelial | 0.657 | 5.98E−05 |
| Spiroplasma | CXCL5 | Endothelial | 0.733 | 9.17E−06 |
| Spiroplasma | GPR110 | Endothelial | 0.654 | 8.89E−05 |
| Spiroplasma | LINC00035 | Endothelial | 0.662 | 9.06E−05 |
| Spiroplasma | DNAJC12 | Endothelial | 0.719 | 7.45E−06 |
| Spiroplasma | CCND1 | Endothelial | −0.654 | 4.95E−05 |
| Spiroplasma | KCNN4 | Endothelial | 0.648 | 8.19E−05 |
| Staphylococcus | NOX5 | Endothelial | −0.652 | 9.47E−05 |
| Streptococcus | CD8A | Endothelial | 0.669 | 1.52E−05 |
| Streptococcus | CCND1 | Endothelial | −0.654 | 2.08E−05 |
| Streptococcus | KLRD1 | Endothelial | 0.669 | 1.51E−05 |
| Streptococcus | PLXDC1 | Endothelial | 0.653 | 2.11E−05 |
| Streptomyces | CADM3 | Endothelial | 0.625 | 9.97E−05 |
| Streptomyces | SPTSSB | Endothelial | 0.646 | 8.69E−05 |
| Streptomyces | HOPX | Endothelial | 0.626 | 9.70E−05 |
| Streptomyces | HPGD | Endothelial | 0.717 | 5.63E−06 |
| Streptomyces | PITX1 | Endothelial | 0.707 | 2.63E−05 |
| Streptomyces | GPR110 | Endothelial | 0.659 | 4.11E−05 |
| Streptomyces | PKIB | Endothelial | 0.662 | 2.77E−05 |
| Streptomyces | ANKRD22 | Endothelial | 0.645 | 6.63E−05 |
| Streptomyces | MUC5B | Endothelial | 0.650 | 7.46E−05 |
| Streptomyces | CCND1 | Endothelial | −0.715 | 2.06E−06 |
| Streptomyces | KLRD1 | Endothelial | 0.640 | 6.05E−05 |
| Streptomyces | PHGR1 | Endothelial | 0.714 | 1.36E−05 |
| Streptomyces | ONECUT3 | Endothelial | 0.656 | 4.50E−05 |
| Streptomyces | CEACAM6 | Endothelial | 0.661 | 2.11E−05 |
| Streptomyces | KCNN4 | Endothelial | 0.642 | 5.59E−05 |
| Vibrio | CD2 | Endothelial | 0.695 | 2.02E−05 |
| Vibrio | GZMA | Endothelial | 0.716 | 5.82E−06 |
| Vibrio | IFITM1 | Endothelial | 0.673 | 3.40E−05 |
| Vibrio | PTPRCAP | Endothelial | 0.664 | 4.63E−05 |
| Vibrio | AMICA1 | Endothelial | 0.744 | 1.59E−06 |
| Vibrio | CD3D | Endothelial | 0.708 | 8.39E−06 |
| Vibrio | LAG3 | Endothelial | 0.694 | 2.94E−05 |
| Vibrio | CD163 | Endothelial | 0.666 | 5.80E−05 |
| Vibrio | KLRB1 | Endothelial | 0.682 | 2.35E−05 |
| Vibrio | CD7 | Endothelial | 0.676 | 2.99E−05 |
| Vibrio | NKG7 | Endothelial | 0.702 | 1.07E−05 |
| Aspergillus | ALCAM | Endothelial | 0.665 | 4.51E−05 |
| Aspergillus | KCNN4 | Endothelial | 0.666 | 5.80E−05 |
| Colletotrichum | ALCAM | Endothelial | 0.695 | 1.03E−05 |
| Colletotrichum | RP11.290F20.3 | Endothelial | 0.665 | 8.27E−05 |
| Saccharomyces | ALCAM | Endothelial | 0.696 | 9.69E−06 |
| Saccharomyces | RP11.290F20.3 | Endothelial | 0.664 | 8.62E−05 |
| Saccharomyces | KCNN4 | Endothelial | 0.649 | 7.86E−05 |
| Thermothielavioides | ALCAM | Endothelial | 0.692 | 1.13E−05 |
| Acinetobacter | CEACAM7 | Fibroblast | −0.801 | 3.82E−05 |
| Bacillus | ASPM | Fibroblast | −0.727 | 8.65E−05 |
| Bacteroides | CD53 | Fibroblast | 0.661 | 9.35E−05 |
| Bacteroides | CTSS | Fibroblast | 0.672 | 8.99E−05 |
| Bacteroides | SELL | Fibroblast | 0.743 | 9.10E−06 |
| Bacteroides | HTRA3 | Fibroblast | 0.733 | 6.06E−06 |
| Bacteroides | UBD | Fibroblast | 0.714 | 2.02E−05 |
| Bacteroides | UCP2 | Fibroblast | 0.728 | 1.69E−05 |
| Bacteroides | GPR183 | Fibroblast | 0.686 | 5.62E−05 |
| Bacteroides | ITGA3 | Fibroblast | 0.689 | 9.86E−05 |
| Burkholderia | RGS4 | Fibroblast | −0.743 | 3.17E−05 |
| Burkholderia | G0S2 | Fibroblast | 0.719 | 7.50E−05 |
| Klebsiella | RGS4 | Fibroblast | −0.724 | 6.33E−05 |
| Klebsiella | AKR1C2 | Fibroblast | 0.719 | 3.44E−05 |
| Megamonas | UCP2 | Fibroblast | 0.725 | 2.80E−05 |
| Megamonas | KLK11 | Fibroblast | 0.785 | 2.09E−06 |
| Megamonas | KCNJ6 | Fibroblast | 0.799 | 2.80E−06 |
| Paracoccus | RGS4 | Fibroblast | −0.722 | 6.72E−05 |
| Pasteurella | AKR1C2 | Fibroblast | 0.761 | 6.51E−06 |
| Prevotella | UCP2 | Fibroblast | 0.781 | 2.53E−06 |
| Prevotella | CD27 | Fibroblast | 0.692 | 6.40E−05 |
| Prevotella | CST4 | Fibroblast | 0.692 | 8.96E−05 |
| Prevotella | KLK11 | Fibroblast | 0.712 | 4.59E−05 |
| Prevotella | KCNJ6 | Fibroblast | 0.721 | 6.95E−05 |
| Sphingobacterium | MACC1 | Fibroblast | 0.689 | 7.13E−05 |
| Staphylococcus | RGS4 | Fibroblast | −0.731 | 4.95E−05 |
| Streptomyces | GJA5 | Fibroblast | −0.683 | 6.10E−05 |
| Streptomyces | CYTL1 | Fibroblast | −0.702 | 9.17E−05 |
| Kluyveromyces | TSPAN1 | Fibroblast | 0.709 | 5.01E−05 |
| Kluyveromyces | HIST2H2AA3 | Fibroblast | 0.761 | 9.84E−06 |
| Kluyveromyces | IL1RN | Fibroblast | 0.692 | 6.36E−05 |
| Kluyveromyces | TIGIT | Fibroblast | 0.714 | 4.19E−05 |
| Kluyveromyces | AREG | Fibroblast | 0.709 | 7.29E−05 |
| Kluyveromyces | PITX1 | Fibroblast | 0.729 | 2.43E−05 |
| Kluyveromyces | LINC00035 | Fibroblast | 0.728 | 5.52E−05 |
| Kluyveromyces | CYBB | Fibroblast | 0.683 | 6.31E−05 |
| Kluyveromyces | PHLDA2 | Fibroblast | 0.688 | 5.21E−05 |
| Kluyveromyces | CTSW | Fibroblast | 0.685 | 8.01E−05 |
| Kluyveromyces | TAGLN | Fibroblast | 0.716 | 1.81E−05 |
| Kluyveromyces | ITGA5 | Fibroblast | 0.722 | 2.16E−05 |
| Kluyveromyces | OASL | Fibroblast | 0.690 | 4.94E−05 |
| Kluyveromyces | GREM1 | Fibroblast | 0.690 | 4.86E−05 |
| Kluyveromyces | C15orf48 | Fibroblast | 0.757 | 1.18E−05 |
| Kluyveromyces | SLC16A3 | Fibroblast | 0.726 | 1.79E−05 |
| Thermothielavioides | CDC20 | Fibroblast | 0.712 | 4.49E−05 |
| Bacteroides | CAPN8 | Macrophage | 0.715 | 5.85E−05 |
| Bacteroides | ANXA10 | Macrophage | 0.737 | 4.03E−05 |
| Klebsiella | KLRC1 | Macrophage | −0.703 | 8.82E−05 |
| Mycoplasma | KLRC1 | Macrophage | 0.673 | 8.69E−05 |
| Pasteurella | KLRC1 | Macrophage | −0.712 | 6.62E−05 |
| Ralstonia | KLRC1 | Macrophage | −0.739 | 5.70E−05 |
| Ralstonia | CD7 | Macrophage | −0.754 | 3.26E−05 |
| Bacteroides | AQP3 | Stellate | 0.710 | 6.94E−05 |
| Burkholderia | F3 | Stellate | −0.667 | 7.81E−05 |
| Burkholderia | FAM150B | Stellate | 0.709 | 3.46E−05 |
| Burkholderia | PDLIM3 | Stellate | −0.687 | 5.41E−05 |
| Burkholderia | CFTR | Stellate | 0.751 | 4.13E−06 |
| Burkholderia | GIMAP5 | Stellate | 0.673 | 8.69E−05 |
| Burkholderia | CERCAM | Stellate | −0.683 | 8.78E−05 |
| Burkholderia | FXYD2 | Stellate | 0.720 | 1.09E−05 |
| Burkholderia | MMP19 | Stellate | −0.678 | 7.36E−05 |
| Burkholderia | CCT2 | Stellate | −0.727 | 7.91E−06 |
| Burkholderia | EGLN3 | Stellate | −0.776 | 8.49E−06 |
| Burkholderia | FAM83D | Stellate | −0.692 | 8.91E−05 |
| Burkholderia | KLK10 | Stellate | −0.672 | 8.92E−05 |
| Burkholderia | TFF2 | Stellate | −0.709 | 5.01E−05 |
| Burkholderia | PNLIPRP1 | Stellate | 0.711 | 9.87E−05 |
| Burkholderia | CTRB2 | Stellate | 0.665 | 8.28E−05 |
| Chryseobacterium | PDIA2 | Stellate | 0.757 | 1.19E−05 |
| Flavobacterium | UGT2A3 | Stellate | 0.725 | 6.15E−05 |
| Flavobacterium | PDIA2 | Stellate | 0.761 | 1.01E−05 |
| Klebsiella | FAM150B | Stellate | 0.720 | 2.28E−05 |
| Klebsiella | GALNT5 | Stellate | −0.697 | 5.31E−05 |
| Klebsiella | PDLIM3 | Stellate | −0.707 | 2.64E−05 |
| Klebsiella | ACHE | Stellate | −0.671 | 9.23E−05 |
| Klebsiella | CFTR | Stellate | 0.719 | 1.64E−05 |
| Klebsiella | CERCAM | Stellate | −0.688 | 7.30E−05 |
| Klebsiella | FXYD2 | Stellate | 0.715 | 1.30E−05 |
| Klebsiella | MMP19 | Stellate | −0.706 | 2.71E−05 |
| Klebsiella | EGLN3 | Stellate | −0.807 | 1.87E−06 |
| Klebsiella | KLK10 | Stellate | −0.680 | 6.81E−05 |
| Klebsiella | PNLIPRP1 | Stellate | 0.719 | 7.56E−05 |
| Klebsiella | CTRB2 | Stellate | 0.661 | 9.44E−05 |
| Megamonas | MOXD1 | Stellate | 0.704 | 4.13E−05 |
| Megamonas | FGF7 | Stellate | 0.742 | 9.47E−06 |
| Megamonas | APOE | Stellate | 0.694 | 5.98E−05 |
| Mycoplasma | PDIA2 | Stellate | 0.724 | 6.27E−05 |
| Paracoccus | TNC | Stellate | −0.710 | 3.37E−05 |
| Paracoccus | PNLIPRP1 | Stellate | 0.712 | 9.59E−05 |
| Pasteurella | F3 | Stellate | −0.664 | 8.62E−05 |
| Pasteurella | HSD11B1 | Stellate | −0.725 | 1.92E−05 |
| Pasteurella | FAM150B | Stellate | 0.723 | 2.08E−05 |
| Pasteurella | GALNT5 | Stellate | −0.685 | 7.96E−05 |
| Pasteurella | PDLIM3 | Stellate | −0.715 | 1.88E−05 |
| Pasteurella | ACHE | Stellate | −0.675 | 8.27E−05 |
| Pasteurella | CFTR | Stellate | 0.734 | 8.74E−06 |
| Pasteurella | GIMAP5 | Stellate | 0.670 | 9.68E−05 |
| Pasteurella | PLAT | Stellate | −0.694 | 4.20E−05 |
| Pasteurella | DKK3 | Stellate | −0.671 | 6.79E−05 |
| Pasteurella | ANO1 | Stellate | 0.683 | 4.41E−05 |
| Pasteurella | FXYD2 | Stellate | 0.740 | 4.42E−06 |
| Pasteurella | MMP19 | Stellate | −0.707 | 2.60E−05 |
| Pasteurella | CCT2 | Stellate | −0.691 | 3.31E−05 |
| Pasteurella | EGLN3 | Stellate | −0.815 | 1.22E−06 |
| Pasteurella | SERPINA5 | Stellate | 0.710 | 2.29E−05 |
| Pasteurella | KLK10 | Stellate | −0.682 | 6.37E−05 |
| Pasteurella | TFF2 | Stellate | −0.727 | 2.60E−05 |
| Pasteurella | CTRB2 | Stellate | 0.667 | 7.74E−05 |
| Prevotella | KLRC1 | Stellate | 0.838 | 2.08E−06 |
| Ralstonia | HSD11B1 | Stellate | −0.702 | 4.46E−05 |
| Ralstonia | CTRB2 | Stellate | 0.672 | 6.60E−05 |
| Spiroplasma | TUBA1A | Stellate | −0.715 | 4.10E−05 |
| Staphylococcus | CFTR | Stellate | 0.680 | 6.96E−05 |
| Staphylococcus | FXYD2 | Stellate | 0.674 | 6.06E−05 |
| Staphylococcus | CCT2 | Stellate | −0.660 | 9.93E−05 |
| Staphylococcus | EGLN3 | Stellate | −0.754 | 2.13E−05 |
| Staphylococcus | FAM83D | Stellate | −0.689 | 9.99E−05 |
| Staphylococcus | CTRB2 | Stellate | 0.666 | 8.01E−05 |
| Streptomyces | PDIA2 | Stellate | 0.745 | 1.93E−05 |
| Aspergillus | ISG15 | Stellate | 0.660 | 9.94E−05 |
| Aspergillus | CDCA8 | Stellate | 0.709 | 2.41E−05 |
| Aspergillus | F3 | Stellate | 0.707 | 1.79E−05 |
| Aspergillus | ECM1 | Stellate | 0.672 | 9.09E−05 |
| Aspergillus | NUF2 | Stellate | 0.775 | 2.09E−06 |
| Aspergillus | UBE2T | Stellate | 0.721 | 1.00E−05 |
| Aspergillus | CD55 | Stellate | 0.692 | 3.21E−05 |
| Aspergillus | FAM150B | Stellate | −0.815 | 2.20E−07 |
| Aspergillus | REG1A | Stellate | −0.676 | 5.60E−05 |
| Aspergillus | SCTR | Stellate | −0.753 | 3.32E−05 |
| Aspergillus | COL5A2 | Stellate | 0.692 | 3.24E−05 |
| Aspergillus | FN1 | Stellate | 0.688 | 3.72E−05 |
| Aspergillus | FBLN2 | Stellate | 0.687 | 3.88E−05 |
| Aspergillus | FAM107A | Stellate | −0.693 | 8.68E−05 |
| Aspergillus | CXCL5 | Stellate | 0.710 | 7.09E−05 |
| Aspergillus | EREG | Stellate | 0.713 | 2.96E−05 |
| Aspergillus | PDLIM3 | Stellate | 0.810 | 1.78E−07 |
| Aspergillus | SPARC | Stellate | 0.718 | 1.17E−05 |
| Aspergillus | AQP1 | Stellate | −0.679 | 7.11E−05 |
| Aspergillus | AEBP1 | Stellate | 0.696 | 2.80E−05 |
| Aspergillus | CFTR | Stellate | −0.778 | 1.11E−06 |
| Aspergillus | CALD1 | Stellate | 0.702 | 6.46E−05 |
| Aspergillus | GIMAP5 | Stellate | −0.764 | 2.19E−06 |
| Aspergillus | EGFL6 | Stellate | 0.741 | 4.27E−06 |
| Aspergillus | LOXL2 | Stellate | 0.750 | 2.84E−06 |
| Aspergillus | SULF1 | Stellate | 0.722 | 1.46E−05 |
| Aspergillus | FABP4 | Stellate | −0.671 | 6.77E−05 |
| Aspergillus | SDC2 | Stellate | 0.703 | 2.08E−05 |
| Aspergillus | CERCAM | Stellate | 0.702 | 4.49E−05 |
| Aspergillus | AKR1C3 | Stellate | 0.671 | 6.75E−05 |
| Aspergillus | CUZD1 | Stellate | −0.704 | 8.61E−05 |
| Aspergillus | SERPINH1 | Stellate | 0.667 | 7.69E−05 |
| Aspergillus | FXYD2 | Stellate | −0.771 | 9.76E−07 |
| Aspergillus | TUBA1C | Stellate | 0.679 | 5.18E−05 |
| Aspergillus | CCT2 | Stellate | 0.744 | 3.78E−06 |
| Aspergillus | COL4A1 | Stellate | 0.707 | 1.78E−05 |
| Aspergillus | COL4A2 | Stellate | 0.712 | 1.50E−05 |
| Aspergillus | EGLN3 | Stellate | 0.721 | 7.02E−05 |
| Aspergillus | LGALS3 | Stellate | 0.672 | 6.60E−05 |
| Aspergillus | LGMN | Stellate | 0.723 | 2.04E−05 |
| Aspergillus | SERPINA5 | Stellate | −0.748 | 4.67E−06 |
| Aspergillus | CDH11 | Stellate | 0.679 | 5.12E−05 |
| Aspergillus | HSD11B2 | Stellate | 0.671 | 9.37E−05 |
| Aspergillus | KPNA2 | Stellate | 0.671 | 6.89E−05 |
| Aspergillus | TK1 | Stellate | 0.672 | 6.47E−05 |
| Aspergillus | TPX2 | Stellate | 0.722 | 9.90E−06 |
| Aspergillus | FAM83D | Stellate | 0.841 | 7.61E−08 |
| Aspergillus | RCN3 | Stellate | 0.694 | 8.47E−05 |
| Aspergillus | KLK10 | Stellate | 0.712 | 2.14E−05 |
| Aspergillus | CTRB2 | Stellate | −0.735 | 5.57E−06 |
| Colletotrichum | ISG15 | Stellate | 0.676 | 5.60E−05 |
| Colletotrichum | CDCA8 | Stellate | 0.721 | 1.52E−05 |
| Colletotrichum | F3 | Stellate | 0.706 | 1.86E−05 |
| Colletotrichum | RP11.14N7.2 | Stellate | 0.673 | 8.84E−05 |
| Colletotrichum | ECM1 | Stellate | 0.672 | 9.09E−05 |
| Colletotrichum | S100A4 | Stellate | 0.662 | 9.16E−05 |
| Colletotrichum | NUF2 | Stellate | 0.773 | 2.30E−06 |
| Colletotrichum | UBE2T | Stellate | 0.723 | 9.24E−06 |
| Colletotrichum | CD55 | Stellate | 0.698 | 2.57E−05 |
| Colletotrichum | FAM150B | Stellate | −0.825 | 1.17E−07 |
| Colletotrichum | REG1A | Stellate | −0.675 | 5.90E−05 |
| Colletotrichum | SCTR | Stellate | −0.767 | 1.93E−05 |
| Colletotrichum | COL5A2 | Stellate | 0.702 | 2.23E−05 |
| Colletotrichum | FN1 | Stellate | 0.698 | 2.52E−05 |
| Colletotrichum | FBLN2 | Stellate | 0.692 | 3.17E−05 |
| Colletotrichum | FAM107A | Stellate | −0.704 | 5.96E−05 |
| Colletotrichum | SMC4 | Stellate | 0.682 | 6.49E−05 |
| Colletotrichum | CXCL5 | Stellate | 0.718 | 5.24E−05 |
| Colletotrichum | EREG | Stellate | 0.708 | 3.61E−05 |
| Colletotrichum | PDLIM3 | Stellate | 0.811 | 1.67E−07 |
| Colletotrichum | VCAN | Stellate | 0.665 | 8.41E−05 |
| Colletotrichum | SPARC | Stellate | 0.727 | 8.02E−06 |
| Colletotrichum | AQP1 | Stellate | −0.677 | 7.66E−05 |
| Colletotrichum | AEBP1 | Stellate | 0.709 | 1.70E−05 |
| Colletotrichum | COL1A2 | Stellate | 0.665 | 8.22E−05 |
| Colletotrichum | CFTR | Stellate | −0.781 | 9.17E−07 |
| Colletotrichum | CALD1 | Stellate | 0.692 | 8.96E−05 |
| Colletotrichum | GIMAP5 | Stellate | −0.762 | 2.51E−06 |
| Colletotrichum | EGFL6 | Stellate | 0.747 | 3.31E−06 |
| Colletotrichum | LOXL2 | Stellate | 0.759 | 1.84E−06 |
| Colletotrichum | SULF1 | Stellate | 0.728 | 1.14E−05 |
| Colletotrichum | FABP4 | Stellate | −0.668 | 7.63E−05 |
| Colletotrichum | SDC2 | Stellate | 0.712 | 1.49E−05 |
| Colletotrichum | CERCAM | Stellate | 0.708 | 3.59E−05 |
| Colletotrichum | AKR1C3 | Stellate | 0.685 | 4.21E−05 |
| Colletotrichum | CUZD1 | Stellate | −0.707 | 7.65E−05 |
| Colletotrichum | SERPINH1 | Stellate | 0.675 | 5.85E−05 |
| Colletotrichum | FXYD2 | Stellate | −0.773 | 9.02E−07 |
| Colletotrichum | TUBA1C | Stellate | 0.688 | 3.76E−05 |
| Colletotrichum | CCT2 | Stellate | 0.753 | 2.48E−06 |
| Colletotrichum | COL4A1 | Stellate | 0.713 | 1.43E−05 |
| Colletotrichum | COL4A2 | Stellate | 0.711 | 1.54E−05 |
| Colletotrichum | EGLN3 | Stellate | 0.717 | 8.17E−05 |
| Colletotrichum | LGALS3 | Stellate | 0.671 | 6.72E−05 |
| Colletotrichum | LGMN | Stellate | 0.738 | 1.12E−05 |
| Colletotrichum | SERPINA5 | Stellate | −0.752 | 3.91E−06 |
| Colletotrichum | CDH11 | Stellate | 0.689 | 3.58E−05 |
| Colletotrichum | KPNA2 | Stellate | 0.675 | 5.80E−05 |
| Colletotrichum | TK1 | Stellate | 0.691 | 3.29E−05 |
| Colletotrichum | TPX2 | Stellate | 0.730 | 7.04E−06 |
| Colletotrichum | FAM83D | Stellate | 0.853 | 3.18E−08 |
| Colletotrichum | PLAUR | Stellate | 0.676 | 7.91E−05 |
| Colletotrichum | RCN3 | Stellate | 0.706 | 5.66E−05 |
| Colletotrichum | KLK10 | Stellate | 0.717 | 1.80E−05 |
| Colletotrichum | CTRB2 | Stellate | −0.730 | 7.08E−06 |
| Kluyveromyces | ISG15 | Stellate | 0.715 | 8.50E−05 |
| Kluyveromyces | CTSS | Stellate | 0.722 | 9.94E−05 |
| Kluyveromyces | S100A4 | Stellate | 0.714 | 9.02E−05 |
| Kluyveromyces | NUF2 | Stellate | 0.816 | 1.16E−06 |
| Kluyveromyces | UBE2T | Stellate | 0.767 | 1.22E−05 |
| Kluyveromyces | FAM150B | Stellate | −0.808 | 5.38E−06 |
| Kluyveromyces | CYS1 | Stellate | −0.748 | 6.17E−05 |
| Kluyveromyces | HK2 | Stellate | 0.742 | 5.06E−05 |
| Kluyveromyces | IL1RN | Stellate | 0.775 | 1.39E−05 |
| Kluyveromyces | FN1 | Stellate | 0.769 | 1.13E−05 |
| Kluyveromyces | CCNA2 | Stellate | 0.784 | 9.50E−06 |
| Kluyveromyces | SLC7A11 | Stellate | 0.755 | 3.09E−05 |
| Kluyveromyces | VCAN | Stellate | 0.729 | 5.40E−05 |
| Kluyveromyces | DLX5 | Stellate | 0.773 | 1.54E−05 |
| Kluyveromyces | CFTR | Stellate | −0.791 | 6.86E−06 |
| Kluyveromyces | GIMAP5 | Stellate | −0.809 | 2.97E−06 |
| Kluyveromyces | EGFL6 | Stellate | 0.785 | 5.57E−06 |
| Kluyveromyces | LOXL2 | Stellate | 0.749 | 2.53E−05 |
| Kluyveromyces | SULF1 | Stellate | 0.724 | 6.33E−05 |
| Kluyveromyces | SDC2 | Stellate | 0.729 | 5.36E−05 |
| Kluyveromyces | TSTA3 | Stellate | 0.748 | 6.26E−05 |
| Kluyveromyces | AKR1C3 | Stellate | 0.798 | 3.02E−06 |
| Kluyveromyces | SFTA1P | Stellate | 0.770 | 1.76E−05 |
| Kluyveromyces | COL17A1 | Stellate | 0.796 | 3.25E−06 |
| Kluyveromyces | FXYD2 | Stellate | −0.791 | 4.27E−06 |
| Kluyveromyces | CDCA3 | Stellate | 0.753 | 2.16E−05 |
| Kluyveromyces | MGST1 | Stellate | 0.717 | 8.08E−05 |
| Kluyveromyces | OASL | Stellate | 0.768 | 1.91E−05 |
| Kluyveromyces | COL4A1 | Stellate | 0.765 | 1.36E−05 |
| Kluyveromyces | COL4A2 | Stellate | 0.782 | 6.52E−06 |
| Kluyveromyces | SERPINA5 | Stellate | −0.809 | 2.94E−06 |
| Kluyveromyces | DUOX2 | Stellate | 0.759 | 2.72E−05 |
| Kluyveromyces | DUOXA2 | Stellate | 0.811 | 8.02E−06 |
| Kluyveromyces | C15orf48 | Stellate | 0.818 | 5.92E−06 |
| Kluyveromyces | CDH11 | Stellate | 0.747 | 2.70E−05 |
| Kluyveromyces | COTL1 | Stellate | 0.762 | 1.52E−05 |
| Kluyveromyces | IRF8 | Stellate | 0.769 | 1.78E−05 |
| Kluyveromyces | CDT1 | Stellate | 0.772 | 1.62E−05 |
| Kluyveromyces | CCL18 | Stellate | 0.734 | 6.79E−05 |
| Kluyveromyces | LINC00671 | Stellate | −0.779 | 1.95E−05 |
| Kluyveromyces | HN1 | Stellate | 0.726 | 5.89E−05 |
| Kluyveromyces | TK1 | Stellate | 0.739 | 3.67E−05 |
| Kluyveromyces | TYMS | Stellate | 0.732 | 4.76E−05 |
| Kluyveromyces | PMAIP1 | Stellate | 0.842 | 9.16E−07 |
| Kluyveromyces | TPX2 | Stellate | 0.787 | 5.15E−06 |
| Kluyveromyces | FAM83D | Stellate | 0.814 | 2.26E−06 |
| Kluyveromyces | RP11.290F20.3 | Stellate | 0.809 | 5.24E−06 |
| Saccharomyces | F3 | Stellate | 0.685 | 5.71E−05 |
| Saccharomyces | S100A4 | Stellate | 0.671 | 9.43E−05 |
| Saccharomyces | NUF2 | Stellate | 0.773 | 3.67E−06 |
| Saccharomyces | UBE2T | Stellate | 0.683 | 6.23E−05 |
| Saccharomyces | CD55 | Stellate | 0.770 | 1.66E−06 |
| Saccharomyces | FAM150B | Stellate | −0.805 | 7.16E−07 |
| Saccharomyces | MXD1 | Stellate | 0.696 | 7.78E−05 |
| Saccharomyces | REG1A | Stellate | −0.676 | 7.99E−05 |
| Saccharomyces | SCTR | Stellate | −0.754 | 5.01E−05 |
| Saccharomyces | COL5A2 | Stellate | 0.678 | 7.34E−05 |
| Saccharomyces | FN1 | Stellate | 0.683 | 6.25E−05 |
| Saccharomyces | FBLN2 | Stellate | 0.700 | 3.34E−05 |
| Saccharomyces | SMC4 | Stellate | 0.693 | 6.15E−05 |
| Saccharomyces | PDLIM3 | Stellate | 0.780 | 1.64E−06 |
| Saccharomyces | VCAN | Stellate | 0.671 | 9.23E−05 |
| Saccharomyces | SPARC | Stellate | 0.700 | 3.32E−05 |
| Saccharomyces | DCDC2 | Stellate | −0.693 | 8.59E−05 |
| Saccharomyces | AEBP1 | Stellate | 0.676 | 7.91E−05 |
| Saccharomyces | CFTR | Stellate | −0.807 | 3.75E−07 |
| Saccharomyces | GIMAP5 | Stellate | −0.705 | 3.95E−05 |
| Saccharomyces | EGFL6 | Stellate | 0.727 | 1.16E−05 |
| Saccharomyces | LOXL2 | Stellate | 0.736 | 8.23E−06 |
| Saccharomyces | SULF1 | Stellate | 0.719 | 1.64E−05 |
| Saccharomyces | SDC2 | Stellate | 0.685 | 5.79E−05 |
| Saccharomyces | FXYD2 | Stellate | −0.729 | 1.06E−05 |
| Saccharomyces | CCT2 | Stellate | 0.720 | 1.54E−05 |
| Saccharomyces | COL4A1 | Stellate | 0.675 | 8.14E−05 |
| Saccharomyces | COL4A2 | Stellate | 0.673 | 8.84E−05 |
| Saccharomyces | LGALS3 | Stellate | 0.669 | 9.78E−05 |
| Saccharomyces | LGMN | Stellate | 0.696 | 7.99E−05 |
| Saccharomyces | SERPINA5 | Stellate | −0.714 | 2.91E−05 |
| Saccharomyces | CDH11 | Stellate | 0.676 | 7.81E−05 |
| Saccharomyces | TPX2 | Stellate | 0.680 | 6.85E−05 |
| Saccharomyces | FAM83D | Stellate | 0.840 | 7.99E−08 |
| Saccharomyces | PLAUR | Stellate | 0.699 | 4.96E−05 |
| Saccharomyces | KLK10 | Stellate | 0.702 | 4.52E−05 |
| Saccharomyces | CTRB2 | Stellate | −0.720 | 1.58E−05 |
| Thermothielavioides | CDCA8 | Stellate | 0.727 | 1.15E−05 |
| Thermothielavioides | F3 | Stellate | 0.691 | 3.36E−05 |
| Thermothielavioides | NUF2 | Stellate | 0.763 | 3.75E−06 |
| Thermothielavioides | UBE2T | Stellate | 0.694 | 2.97E−05 |
| Thermothielavioides | CD55 | Stellate | 0.668 | 7.50E−05 |
| Thermothielavioides | FAM150B | Stellate | −0.807 | 3.71E−07 |
| Thermothielavioides | REG1A | Stellate | −0.676 | 5.70E−05 |
| Thermothielavioides | SCTR | Stellate | −0.760 | 2.54E−05 |
| Thermothielavioides | COL5A2 | Stellate | 0.684 | 4.34E−05 |
| Thermothielavioides | FN1 | Stellate | 0.677 | 5.51E−05 |
| Thermothielavioides | FBLN2 | Stellate | 0.685 | 4.10E−05 |
| Thermothielavioides | FAM107A | Stellate | −0.698 | 7.46E−05 |
| Thermothielavioides | CXCL5 | Stellate | 0.732 | 3.18E−05 |
| Thermothielavioides | EREG | Stellate | 0.699 | 5.05E−05 |
| Thermothielavioides | PDLIM3 | Stellate | 0.797 | 3.96E−07 |
| Thermothielavioides | SPARC | Stellate | 0.692 | 3.16E−05 |
| Thermothielavioides | AEBP1 | Stellate | 0.719 | 1.11E−05 |
| Thermothielavioides | CFTR | Stellate | −0.773 | 1.39E−06 |
| Thermothielavioides | GIMAP5 | Stellate | −0.743 | 5.83E−06 |
| Thermothielavioides | EGFL6 | Stellate | 0.740 | 4.57E−06 |
| Thermothielavioides | LOXL2 | Stellate | 0.731 | 6.72E−06 |
| Thermothielavioides | SULF1 | Stellate | 0.704 | 2.92E−05 |
| Thermothielavioides | SDC2 | Stellate | 0.677 | 5.41E−05 |
| Thermothielavioides | CERCAM | Stellate | 0.689 | 7.06E−05 |
| Thermothielavioides | AKR1C3 | Stellate | 0.681 | 4.86E−05 |
| Thermothielavioides | CUZD1 | Stellate | −0.716 | 5.69E−05 |
| Thermothielavioides | FXYD2 | Stellate | −0.761 | 1.67E−06 |
| Thermothielavioides | CCT2 | Stellate | 0.730 | 6.90E−06 |
| Thermothielavioides | COL4A1 | Stellate | 0.682 | 4.62E−05 |
| Thermothielavioides | COL4A2 | Stellate | 0.684 | 4.34E−05 |
| Thermothielavioides | EGLN3 | Stellate | 0.730 | 5.13E−05 |
| Thermothielavioides | LGMN | Stellate | 0.707 | 3.75E−05 |
| Thermothielavioides | SERPINA5 | Stellate | −0.756 | 3.35E−06 |
| Thermothielavioides | CDH11 | Stellate | 0.667 | 7.74E−05 |
| Thermothielavioides | TK1 | Stellate | 0.668 | 7.42E−05 |
| Thermothielavioides | TPX2 | Stellate | 0.683 | 4.44E−05 |
| Thermothielavioides | FAM83D | Stellate | 0.825 | 2.23E−07 |
| Thermothielavioides | KLK10 | Stellate | 0.692 | 4.49E−05 |
| Thermothielavioides | CTRB2 | Stellate | −0.722 | 9.64E−06 |
| Chryseobacterium | HIST1H4C | T_cell | −0.804 | 9.90E−05 |
| Aspergillus | THBS4 | T_cell | 0.890 | 2.05E−05 |
| Aspergillus | LPL | T_cell | 0.881 | 1.44E−05 |
| Colletotrichum | LPL | T_cell | 0.870 | 5.31E−05 |
| Kluyveromyces | PLA2G2A | T_cell | 0.863 | 3.41E−05 |
| Kluyveromyces | CD34 | T_cell | 0.887 | 2.36E−05 |
| Kluyveromyces | UCHL1 | T_cell | 0.846 | 7.12E−05 |
| Saccharomyces | LPL | T_cell | 0.870 | 5.31E−05 |
| Thermothielavioides | LPL | T_cell | 0.870 | 5.31E−05 |

Microbiome predicted patient survival: Whether intra-tumoral microbial diversity and associated gene expression signatures could predict patients at risk of poor survival was determined. First, pseudo-bulk gene expression profiles were created from the Peng et al. (Peng et al. Cell Res. 29(9): 725-738, 2019) cohort by summing the gene counts across all cells in a given sample. Regularized logistic regression was then used to identify a six-gene signature that accurately classified the samples as having low or high microbial diversity, defined as having a Shannon index below or above the median for the cohort (Example 1, FIG. 5G). Next, the model was used to predict whether individual pancreatic tumors profiled with bulk-RNA sequencing from TCGA (Raphael et al. Cancer Cell, 32: 185-203.e13, 2017) and the International Cancer Genomics Consortium (ICGC) (Hudson et al. Nature, 464: 993-998, 2010) had high or low intra-tumoral microbial diversity. Patients were then stratified by the predicted microbial diversity of their tumor and the relationship with survival was tested using a univariate Cox proportional hazards model (FIGS. 5G-5H). In both datasets, high microbial diversity was associated with significantly decreased overall survival (TCGA: Hazard Ratio [HR]=2.6, 95% Confidence Interval [CI]: 1.4-5.3, p=0.0031; ICGC: HR=1.9, 95% CI: 1.2-2.9, p=0.0053; FIG. 5H). A similar trend was observed when stratifying TCGA patients by microbiome diversity calculated from microbial profiles directly measured from the same samples and reported by Poore et al (Poore et al. Nature, 579: 567-574, 2020), albeit with a smaller effect size (p=0.083, FIG. 5H), highlighting the increased resolution possible when single-cell data are used. Of note, there was a 63% overlap between predicted and observed TCGA diversity. These results indicated that microbial composition and associated gene expression signatures in host-cells can identify PDA patients at risk of poor outcomes, and that the model derived from single cell genomic data outperforms that derived from genomic data from bulk tumor tissues, due to its greater resolving power.

### Example 3—Quality Control Analysis

False-positive identifications are a significant problem in metagenomics classification systems. This example describes a particular embodiment of the SAHMI (Single-cell Analysis of Host-Microbiome Interactions) method to identify microbes and viruses in subjects at single cell resolution using genomic approaches, including criteria for improved identification of true species versus contaminants and false positives. These criteria can be used to reduce the occurrence of false positives and contaminants in any of the methods disclosed herein.

As described in Examples 1 and 2, metagenomic classification of paired-end reads from scRNAseq fastq files was done using Kraken 2 (Wood et al. Genome Biol. 20: 257, 2019). The present example also employed KrakenUniq (Breitwieser et al. Genome Biology. 19:198, 2018), which combines very fast k-mer-based classification with a fast k-mer cardinality estimation. KrakenUniq adds a method for counting the number of unique k-mers identified for each taxon using the cardinality estimation algorithm HyperLogLog. By counting how many of each genome's unique k-mers are covered by reads, KrakenUniq can more effectively discern false-positive from true-positive matches.

To mitigate the influence of classification errors, contamination, and noise, results from Kraken 2 and KrakenUniq analyses were assessed against four criteria for selecting true species in a set of samples and reducing or eliminating false positives and contaminants. Common contaminants and false positive signatures were identified using a wide variety of cell lines. The four criteria were as follows: (1) a true species had a positive relationship between the number of reads assigned and number of minimizers assigned; (2) a true species has a positive relationship between number of reads assigned and number of unique minimizers assigned; (3) a true species has a positive relationship between number of minimizers assigned and number of unique minimizers assigned; and (4) a true species has a fractional composition of the detected microbiomes that is greater than that found in negative controls samples. In the absence of paired negative controls, cell line experiments can be used (wherein only false positives and contaminants would be expected to be found). Microbes and viruses identified using Kraken 2 and KrakenUniq that fit the criteria (i.e., species that were present in samples in greater numbers than in negative controls) were maintained for further processing and analysis. Reads were then deduplicated and demultiplexed based on their cell barcode and unique molecular identifiers, sparse barcodes were filtered out, and barcode taxa reassignment was performed.

Mapped metagenomic reads first underwent a series of filters. ShortRead (Morgan et al. Bioinformatics 25: 2607-2608, 2009) was used to remove low complexity reads (<20 non-sequentially repeated nucleotides), low quality reads (PHRED score<20), and PCR duplicates tagged with the same unique molecular identifier and cellular barcode. Non-sparse cellular barcodes were then selected by using an elbow-plot of barcode rank vs. total reads, smoothed with a moving average of 5, and with a cutoff at a change in slope <10⁻³, in a manner analogous to how cellular barcodes are typically selected in single-cell sequencing data (CellRanger (10× Genomics), Drop-seq Core Computational Protocol v2.0.0 (McCarroll laboratory)). Lastly, taxizedb (Chamberlain et al. Tools for Working with ‘Taxonomic’ Databases, 2020) was used to obtain full taxonomic classifications for all resulting reads, and the number of reads assigned to each clade was counted.

Next, sample-level normalized metagenomic levels were calculated as log2 (counts/total_counts*10,000+1). For analyses that compared cell-level metagenome and somatic gene expression, the default Seurat normalization was used. To identify bacteria, fungi, and viruses that were differentially present in case samples compared to controls, or that were present in both case samples and in positive controls, a linear model was constructed to predict sample-level normalized microbe or virus levels as a function of tissue status, somatic cellular composition (to account for potential tropisms), and total metagenomic reads. Cellular counts and total metagenomic counts were log-normalized prior to model fitting.

### Example 4—Detecting an Infection

This example describes a particular embodiment of the SAHMI (Single-cell Analysis of Host-Microbiome Interactions) method to identify microbes and viruses in subjects at single cell resolution using genomic approaches.

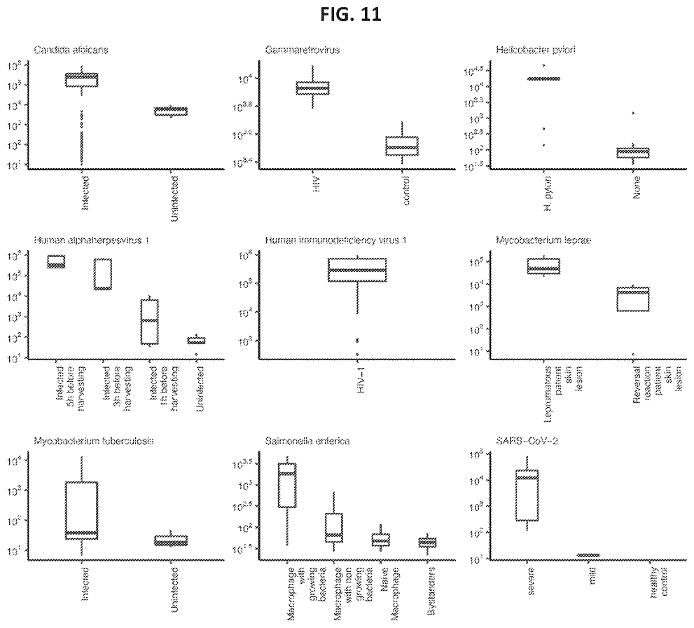

SAHMI was used herein to identify infectious disease agents (e.g., microbes and viruses) using scRNAseq data from various types of human tissues, including blood, skin, stomach, and lung samples. SAHMI identified relevant infectious disease agents in samples as compared to controls for each agent tested (Candida albicans, HIV (with and without controls), Helicobacter pylori, alphaherpesvirus 1, Mycobacterium leprae, Mycobacterium tuberculosis, Salmonella enterica, and SARS-COV-2) (FIG. 11).

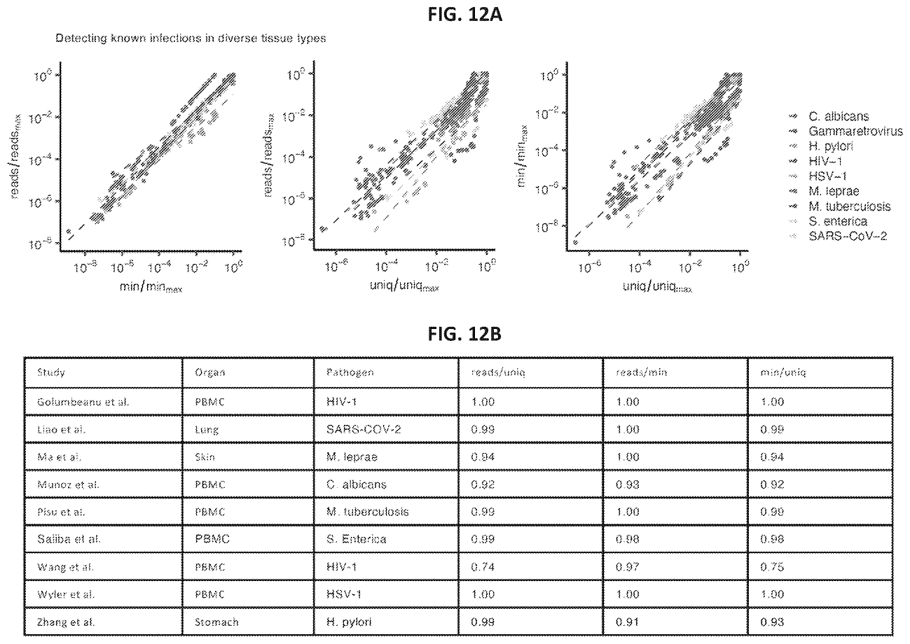

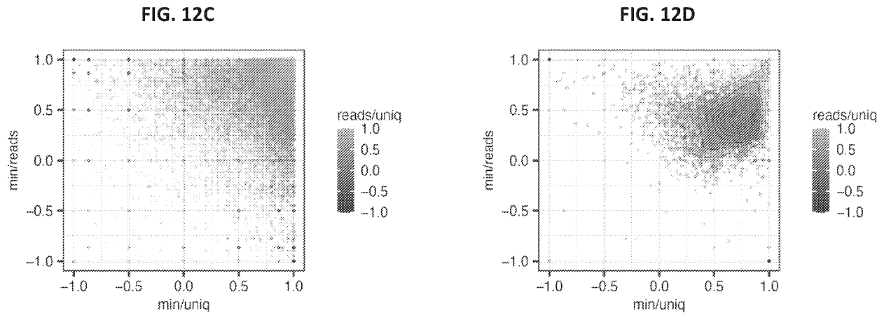

The criteria described in Example 3 were applied for detecting and de-noising the microbiome signals. Sequencing reads from true species had positive relationships between (1) the number of reads assigned and number of minimizers assigned, (2) number of minimizers assigned and number of unique minimizers assigned, and (3) number of reads assigned and number of unique minimizers assigned (FIGS. 12A-12B). Low correlation values for the three criteria indicated the presence of false positive results, whereas high values suggested the presence of other species, including contaminants (FIGS. 12C-12D). In test samples, species not detected above the thresholds found in negative controls (FIG. 12D) were assumed to be false positive or contaminant species.

These data indicate that SAMHI can identify infectious agents, including bacteria, fungi, and viruses, using scRNAseq data from various tissue types collected from subjects that have, or are suspected of having, an infection.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of treating a subject having or suspected of having pancreatic cancer, comprising:
sequencing microbial nucleic acid molecules in individual cells obtained from the subject, wherein the microbes comprise or consist of microbes of genera Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia;
classifying the subject as having pancreatic cancer when the presence of Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, and/or Aspergillus microbes is detected in the individual cells; and
if the subject is determined to have pancreatic cancer, administering at least one of surgery, radiation therapy, a chemotherapeutic agent, antimicrobial, selective bacteriophage, or palliative care to the subject, thereby treating the subject.

2-3. (canceled)

4. A method of determining T-cell microenvironment reaction in a subject, comprising sequencing nucleic acid molecules in individual T-cells obtained from the subject, determining the expression level of one or more of the genes of Table 2 in the individual T-cells, and comparing the expression level of the one or more genes of Table 2 in the individual T-cells to a control using a random forest model, thereby classifying the individual T-cells as infection microenvironment reactive or tumor microenvironment reactive.

5. A method of identifying a microbe or virus in a sample, comprising:
sequencing microbial and/or viral nucleic acid molecules in individual cells obtained from the sample; and
identifying the microbe or the virus in the sample when a microbial or viral nucleic acid indicative of the presence of the microbe or the virus is detected, wherein the identifying further comprises:
(i) mapping reads from a single cell RNA sequencing dataset for the sample to microbial and/or viral genomes using a metagenomics classifier, thereby assigning a genus and/or species identity to each read in the dataset;
(ii) for each genus and/or species identified in (i):
(a) comparing the number of reads assigned and the number of minimizers assigned;
(b) comparing the number of minimizers assigned and the number of unique minimizers assigned; and
(c) comparing the number of reads assigned and the number of unique minimizers assigned; and
(iii) classifying the genus and/or species as a true positive result when a correlation value for each comparison in (ii)(a)-(ii)(c) is positive, and when a number of reads detected for the species is greater in the single cell RNA sequencing dataset as compared to a control.

6. The method of claim 5, wherein the sample is a sample from a subject, and the method further comprises
classifying the subject as having an infectious disease caused by the microbe or the virus, when the microbe or the virus is identified in the sample; and
administering at least one of an antimicrobial, antifungal, or antiviral to the subject;
thereby treating the subject.

7. (canceled)

8. The method of claim 5, wherein
the microbe is a microbe of genera Candida, Helicobacter, Mycobacterium, or Salmonella; or
the virus is a lentivirus, an alphaherpesvirus, or a coronavirus.

9. The method of claim 8, wherein
the microbe of genus Candida is Candida albicans, the microbe of genus Helicobacter is Helicobacter pylori, the microbe of genus Mycobacterium is Mycobacterium leprae or Mycobacterium tuberculosis, or the microbe of genus Salmonella is Salmonella enterica; or
the lentivirus is human immunodeficiency virus, the alphaherpesvirus is alphaherpesvirus-1, or the coronavirus is a betacoronavirus.

10. The method of claim 9, wherein the betacoronavirus is SARS, SARS-CoV, or SARS-COV-2.

11. The method of claim 4 wherein the subject has a cancer.

12. The method of claim 11, wherein the cancer is pancreatic cancer.

13. The method of claim 1, further comprising classifying the subject as not having pancreatic cancer when the presence of Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, and/or Aspergillus microbes is not detected in the individual cells.

14-16. (canceled)

17. The method of claim 1, wherein the chemotherapeutic agent is one or more of gemcitabine, 5-fluorouracil, oxaliplatin, capecitabine, cisplatin, irinotecan, liposomal irinotecan, paclitaxel, albumin-bound paclitaxel, or docetaxel.

18. The method of claim 1, further comprising classifying the subject as having a poor or good survival outcome, the classifying comprising measuring expression of a set of genes in the individual cells obtained from the subject, the set of genes comprising NTHL1, LYPD2, MUC16, C2CD4B, FMO3, and/or IL1RL1.

19. (canceled)

20. The method of claim 18, wherein increased expression of one or more of IL1RL1, C2CD4B, FMO3, or NTHL1 compared to a control, and/or decreased expression of one or more of LYPD2 or MUC16 compared to the control indicates high microbial diversity and classifies the subject as having a poor survival outcome; and/or
wherein decreased expression of one or more of IL1RL1, C2CD4B, FMO3, or NTHL1 compared to a control, and/or increased expression of one or more of LYPD2 or MUC16 compared to the control indicates low microbial diversity and classifies the subject as having a good survival outcome.

21-22. (canceled)

23. The method of claim 18, wherein classifying the subject as having a poor or good survival outcome further comprises calculating the Shannon diversity index for the sample, thereby determining the microbial diversity of the sample.

24. The method of claim 1, wherein the subject does not exhibit symptoms of pancreatic cancer.

25. The method of claim 1, further comprising measuring expression of at least one housekeeping or internal control molecule.

26. The method of claim 1, wherein the individual cells are obtained from tumor tissue, whole blood, serum, or plasma.

27. The method of claim 1, wherein the subject is a human.

28-29. (canceled)

30. The method of claim 5, wherein the correlation value for each comparison is greater than 0.5, greater than 0.7, greater than 0.9, or greater than 0.95.

31-33. (canceled)

34. The method of claim 5, wherein the correlation value is determined using a Spearman correlation.

35-37. (canceled)
